(19) United States
(12) Patent Application Publication
(10) Pub. No.: US 2024/0197965 A1
(43) Pub. Date: Jun. 20, 2024

(54) FOAM BIOMATERIAL AND SYSTEMS, METHODS, AND DEVICES FOR MAKING AND USING SAME

(71) Applicant: University of Connecticut, Farmington, CT (US)

(72) Inventors: Ali Tamayol, Storrs, CT (US); Mohamadmahdi Samandari, Storrs, CT (US); Azadeh Mostafavi, Storrs, CT (US); Nasim Annabi, Storrs, CT (US); Indranil Sinha, Storrs, CT (US); Jacob Quint, Storrs, CT (US); Mehdi Kazemzadeh Narbat, Storrs, CT (US)

(21) Appl. No.: 18/285,602

(22) PCT Filed: Apr. 8, 2022
(86) PCT No.: PCT/US2022/024034

§ 371 Date: Oct. 4, 2023

Related U.S. Application Data
(60) Provisional application No. 63/172,754, filed on Apr. 9, 2021.

(51) Int. Cl. A61L 27/56; A61L 26/00; A61L 27/26

(52) U.S. Cl. CPC A61L 27/56; A61L 26/0052; A61L 26/0085; A61L 27/26; A61L 2300/414; A61L 2430/30

(57) ABSTRACT

A foam biomaterial is disclosed. The foam biomaterial includes a biomaterial ink having an interconnected pore structure. The foam biomaterial can have a porosity from about 10% to about 90%. Systems and methods for forming and depositing the foam biomaterial are also disclosed. In exemplary applications, the foam biomaterial can be deposited on or within a body of a subject or patient.

### CROSS-REFERENCE TO RELATED APPLICATIONS

This application claims priority to U.S. Provisional Patent Application No. 63/172,754, filed Apr. 9, 2021, the entirety of which is hereby incorporated by reference herein.

### FIELD

This disclosure relates to biomaterials and, in particular to foam biomaterials having interconnected pore structures.

### BACKGROUND

Bioprinting has emerged as a promising tool in tissue engineering and regenerative medicine. However, poor cell spreading, proliferation, and migration along with impaired transport of nutrients, oxygen, and waste due to the dense biomaterial networks limit the application of many 3D printable bioinks. Moreover, biomaterials have been injected into injury sites, but injected biomaterials fail to control any spatial organization of the delivered material and is limited to single materials. Traditionally, bioprinted constructs are printed from hydrogels, which possess small pores. Therefore, such bioprinted constructs have a limited diffusion rate and cell infiltration.

Based on the foregoing, a need exists for a bioink with optimal rheological properties and printability as well as proper mechanical properties and porous structure that enable cell infiltration and facile mass transport.

### SUMMARY

Described herein, in various aspects, is a foam biomaterial comprising a biomaterial ink having an interconnected pore structure, wherein the foam biomaterial has a porosity from about 10% to about 90%.

In one aspect, a system for making the foam biomaterial comprises a vessel and a solution in a vessel. The solution comprises a biomaterial. A mixer that is configured to combine the solution with a gas to generate a foam.

In one aspect, a method of making a foam biomaterial as disclosed herein comprises mechanically mixing a solution with a gas to generate a foam, wherein the solution comprises at least one biomaterial. The foam is deposited in a selected location.

Additional advantages of the disclosed compositions, systems, and methods will be set forth in part in the description that follows, and in part will be obvious from the description, or may be learned by practice of the claimed invention. The advantages of the disclosed compositions, systems, and methods will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION OF THE DRAWINGS

These and other features of the preferred embodiments of the invention will become more apparent in the detailed description in which reference is made to the appended drawings wherein:

FIGS. 1(a)-1(b) illustrate the concept of engineering foam bioinks for 3D bioprinting of scaffolds. FIG. 1(a) shows a schematic illustration of 3D bioprinting multiscale porous structure using foam-based bioinks. The preparation process of the cell-laden foam bioink through hydrogel precursor stirring followed by cell addition: (ii) 3D bioprinting of the foam bioink in vitro using a stationary bioprinter or in vivo utilizing a handheld bioprinter: (iii) Multiscale porous structure of the bioprinted foam, facilitating cell expansion in the printed constructs. FIG. 1(b) shows micrographs from hierarchical macro- to micro-scale pores within the 3D printed scaffolds: FIG. 1(b)(i) shows a representative bright-field microscopy image from macroporous 3D printed structure: FIG. 1(b)(ii-iv) show representative SEM images from printed foam bioink filaments, FIG. 1(b)(ii, iii) interconnected mesopores structure formed by foaming process, FIG. 1(b)(iv) microporous Ge1MA structure formed after crosslinking of the polymer chains.

FIG. 2 shows ex vivo printing of the foam bioinks into induced volumetric muscle loss (VML) defect.

FIG. 3 shows wound injury models to show the feasibility of foam in vivo printing and its adhesion to the surrounding tissue.

FIG. 4 shows Live/Dead staining demonstrating the viability of the cells (live: green, dead: red) encapsulated cells in the foam and gel scaffolds at day 1 and 3 post printing.

FIG. 5 shows in vitro results of a PrestoBlue assay, indicating the proliferation profile for cells cultured for 3 days in foam and gel scaffolds.

FIG. 6 illustrates subcutaneous implants in vivo. Both foam and gel scaffolds were implanted subcutaneously in rats and monitored for 4 weeks. Hematoxylin and eosin (H&E) and Masson's trichrome (MT) staining of the scaffold-tissue interface one-week post implantation is shown. Black arrowheads pointing to neovessels formed in the foam scaffold, demonstrating the high level of foam-tissue integration.

FIG. 7 shows the quantitative evaluation of in vivo tissue vascularization in surrounding tissue and inside the implanted scaffold one-week post implantation.

FIGS. 8(a)-8(d) show a characterization of porosity in printed foam constructs. FIG. 8(a) shows tunability of the pore size and distribution within the foam bioink through manipulation of (i) stirring time, (ii) stirring speed (RPM), and (iii) precursor hydrogel concentration. FIG. 8(b) shows the quantitative representation of pore size distribution corresponding to groups shown in Figure (a) (n=3). FIG. 8(c) shows the structural stability of printed foam filaments after 1 h immersion in DPBS at 37° C. Fluorescent images captured from Rhodamine B loaded foam filaments demonstrates the stability of the interconnected pores after extrusion and incubation in physiological condition. FIG. 8(d) Comparison between the density of gel and foam bioinks (n=4). (e) Calculated porosity in foam bioinks made with different Ge1MA concentrations (n=4).

FIG. 9 shows a comparison of stress and strain data for different gel and foam samples.

FIG. 10 shows the mass change measurement of the foam and gel scaffolds in DPBS solution (n=5).

FIGS. 11(a)-11(d) show the physical and mechanical characterization of the foam scaffolds in comparison to gel samples. FIG. 11(a) shows the swelling ratio of the foam and gel scaffolds with different concentrations shows higher swelling of foam scaffolds (n=5). FIG. 11(b) shows the mass change measurement of the foam and gel scaffolds in collagenase type I (20 μg/mL in DPBS) solution to mimic an enzymatic degradation of gelatin-based materials in vivo (n=5). FIG. 11(c) shows the compressive moduli of disk samples fabricated from different foam and gel bioinks (n≥5) and FIG. 11(d) shows their behavior under cyclic compression. The graph shows the material behavior at the 10^{th} loading cycle 10 (except Ge1MA 10% which broke at its first cycle). The inset shows the magnified plots obtained from the loading of foam scaffolds (n=2).

FIG. 12 is a graph showing the Energy loss at 10^{th} loading cycle 10 (except Ge1MA 10% which broke at its first cycle) (n=2).

FIGS. 13(a)-13(d) show 3D printing of foam bioink using stationary and handheld bioprinters. Rheological parameters including FIG. 13(a) storage and loss moduli, as well as FIG. 13(b) bioink viscosity, were compared between the foam and gel bioinks. FIG. 13(c) shows various 3D structures printed with foam bioink using a stationary 3D bioprinter: (i, ii) zigzag and spiral printed patterns and their corresponding designs: (iii) a multi-layered (16 layers) hollow cylinder printed construct as a free-standing 3D structure. FIG. 13(d) shows 3D printing of foam using a handheld printer. (i) The custom-built handheld printer; (ii) a 3D heart shape structure printed with two different foam bioinks, followed by their in situ crosslinking using an embedded blue crosslinking light: (iii) filed syringe with 3 colors of foam bioink which did not blend to each other and continued printed filaments structure from the same syringe to show that ability of gradient printing with foam bioinks.

FIG. 14(a) shows an image of printing a 3-layer spiral structure through an Allevi 3D bioprinter. FIG. 14(b) shows an image from the printed structure. FIG. 14(c) is a table of optimized parameters used for foam printing.

FIGS. 15(a)-15(f) show an evaluation of the foam bioink adhesion to the body tissues. FIG. 15(a) shows lap shear experiment set up, (i) schematic representation of the lap shear test, (ii) foam bioink stretched under shear and ruptured from the bulk foam (cohesion failure mechanism), compared to (iii) the gel samples failed at the tissue-scaffold interface. FIG. 15(b) shows the ultimate adhesion strength of the bioinks to porcine skin. FIG. 15(c) shows toughness and (d) ultimate shear strain of different foam and gel scaffolds, calculated from the lap shear test. FIG. 15(e) shows ex vivo printing of the foam bioinks into induced VML and (f) wound injury models to show the feasibility of foam in vivo printing and its adhesion to the surrounding tissue.

FIGS. 16(a)-16(d) show in vitro and in vivo biocompatibility assessments of the foam and normal gel scaffolds. FIG. 16(a) shows Live/Dead staining demonstrating the viability of the cells (live: green, dead: red) encapsulated cells in the foam and gel scaffolds at day 1 and 3 post printing. FIG. 16(b) shows the results of the PrestoBlue assay indicated the cells proliferation profile cultured for 3 days in foam and gel scaffolds. FIG. 16(c) shows Hematoxylin and eosin (H&E) and Masson's trichrome (MT) staining of the scaffold-tissue interface one-week post implantation. Black arrowheads pointing to neovessels formed in the foam scaffold, demonstrating the high level of foam-tissue integration. FIG. 16(d) shows quantitative evaluation of tissue vascularization in surrounding tissue and inside the implanted scaffold one-week post implantation.

FIG. 17 shows live/Dead micrographs from encapsulated NIH 3T3 cells in foam and gel scaffold after 1 and 3 Days of culture.

FIG. 18(a) shows low magnification images of H&E stained foam and gel samples after one-week post implantation. FIG. 18(b) shows H&E stained images of foam and gel samples after 4 weeks post implantation.

FIGS. 19(A)-19(D) show a murine VML model and an exemplary strategy for its treatment. FIG. 19(A) shows gross images of the extracted muscles 8 weeks post-surgery. Lack of regeneration and smaller volume of the muscle in the VML injury group compared to the sham group confirmed the applicability of the model as a VML injury. FIG. 19(B) shows force generation capability of the muscle post VML injury. A significant reduction was detected in the measured isometric torque immediately after defect induction, as well as eight weeks post-surgery, demonstrating the chronic deficit of the muscle post VML. FIG. 19(C) shows an assessment of IGF-1 level in remnant muscle post VML. A significant reduction in the concentration of IGF-1 was observed two weeks post VML injury. Considering the requirement of IGF-1 in natural muscle regeneration, a reduced level of this growth factor can be considered a major contributor in impaired regeneration post VML. FIG. 19(D) shows an exemplary strategy for the treatment of VML in this work. Negatively charged gelatin nanoparticles are synthesized to link positively charged IGF-1 into a positive Ge1MA structure. The precursor is then foamed and directly printed into the muscle defect using an in situ printing method with a custom handheld printer. The printed scaffolds adhere to the remnant tissue and possess a mesoporous structure to facilitate cell infiltration. The release of IGF-1 then is expected to enhance the activity of infiltrated cells toward muscle regeneration and its functional recovery.

FIGS. 20(A)-20(F) show in vitro effect of IGF-1 on C2C12 muscle progenitors.

FIG. 20(A) shows metabolic activity of myoblasts, exposed to different levels of IGF-1, over one week in culture condition. IGF-1 at a physiological concentration (10 ng/ml) significantly enhanced cellular proliferation. FIG. 20(B) shows F-actin/DAPI staining of the cells exposed to different IGF-1 levels on day 3 and day 7 of culture. Enhanced proliferation and consequent alignment can be observed in cells exposed to 10 ng/ml IGF on day 7. FIGS. 20(C)-20(F) show gene expression analysis of the cellular behavior during differentiation. The expression of two myogenic markers α-actinin (FIG. 20(C)) and MRF4 (FIG. 20(D)) demonstrated a significant improvement in cellular differentiation. The expression of Collagen I (FIG. 20(E)), an ECM protein, and β1-integrin (FIG. 20(F)), a cell adhesion molecule, was further significantly increased during differentiation.

FIGS. 21(A)-21 (I) show the development and characterization of the engineered bioink. FIG. 21(A) shows SEM micrographs demonstrated the multiscale porous structure of the foam bioink and incorporated gelatin microparticles into the structure. (i), (ii), and (iii) show pores in different scales ((i) indicates the foam-induced mesopores and (iii) indicates the inherent micropores). White arrowheads in (ii) show ruptured thin membranes between the bubbles, as a result of foam submersion in saline solution, forming an interconnected mesoporous structure. Yellow arrowheads indicate the adhered gelatin microparticles into the structure. FIG. 21(B) shows a quantitative assessment of different pore sizes in the engineered scaffold. FIG. 21(C) shows an SEM image of gelatin microparticles after synthesis. FIG. 21(D) shows the size of gelatin microparticles measure from SEM micrographs using ImageJ software. FIG. 21(E) shows a release profile of IGF-1 growth factor from microparticles, foam, foam with microparticles, and solid Ge1MA. A sustained release of growth factor for more than a week was detected. The inclusion of microparticles in the foam scaffold slowed the release of IGF-1. FIG. 21(F) shows a compression test for evaluating the mechanical properties of the scaffolds. The test setup is shown schematically on the left, while the results are graphed as compressive modulus on the right. A significant decrease was detected upon foaming, while the incorporation of microparticles did not significantly affect the results. FIG. 21(G) shows an evaluation of scaffold adhesion capability to the tissue. A shear test (left schematic) was used to measure the adhesion of the printed scaffold to the muscle and the results were graphed as ultimate shear strength (right graph). While the adhesion significantly decreased through foaming, all of the scaffolds demonstrated strong adhesion to the tissue as a result of in situ crosslinking. FIG. 21(H) shows a smooth long filament extruded from the nozzle tip demonstrated a high level of foam bioink printability. FIG. 21(I) shows a bright-field micrograph of a printed filament showing the preserved mesoporous structure of the scaffold after printing.

FIGS. 22(A)-22(F) show an application of in situ printing of engineered bioink for murine VML treatment and macroscopic evaluation of its effectiveness. FIG. 22(A) shows the workflow of animal studies in this study as described herein. On day 0, surgeries were performed through induction of VML in GA muscle of both mice legs, and treatments were applied as shown in FIG. 22(B). After 8 weeks, the functional recovery of the legs strength was measured using torque measurement, followed by opening the skin and direct in situ measurement of GA muscle force generation capability. The animals then were sacrificed and tissues were harvested for histology analysis. FIG. 22(B) shows the VML induction and treatment procedure. After opening the skin, the GA muscle was exposed (i) and a 4 mm biopsy punch was implemented (ii) to remove ˜20% of the muscle mass (iii). Then, the engineered bioink was directly printed in VML defect and crosslinked in situ (iv, v). FIG. 22(C) shows a custom handheld printer with an integrated photocrosslinking mechanism that was used for this study. FIG. 22(D) shows gross representative pictures of harvested GA muscle demonstrating the restored volume of the muscle eight weeks post-surgery as a result of in situ printing of engineered scaffold. FIG. 22(E) shows an assessment of functional recovery of the leg strength using torque measurements. Significant functional recovery was detected in VML+Foam+IGF group compared to untreated muscles. FIG. 22(F) shows an evaluation of GA muscle recovery after eight weeks of VML induction through in situ tetanus force measurements. A statistically significant recovery was detected in muscles treated with VML+Foam+IGF compared to untreated muscles.

FIGS. 23(A)-23(G) show the microscopic evaluation of regeneration of VML injury treated with in situ printing of engineered scaffold 8 weeks post-surgery. FIG. 23(A) shows cross sections of the muscle were harvested from the mice and stained using the MT approach. The magnified images of the injury area are provided in FIG. 23(B). MT staining demonstrates a reduced level of fibrosis in both treatment groups compared to VML Untreated group. FIG. 23(C) shows triple-immunofluorescent staining for myofibers basal lamina (Laminin), sarcomere myosin heavy chain (MF20), and embryonic myosin heavy chain (eMHC). Denser and well-oriented muscle fibers with a higher amount of eMHC signal were observed in the injury area of the VML+Foam+IGF samples compared to other groups. FIG. 23(D) shows triple-immunofluorescent staining for Laminin, nuclei (DAPI), and acetylcholine receptor (AchR), a component of neuromuscular junctions. A higher level of AchR signal with nuclei positioned at the border of the fibers in the VML+Foam+IGF group demonstrates a high level of regeneration and muscle maturation.

FIG. 24(A) shows a schematic of an 8-week-long exercise regimen as disclosed herein. FIG. 24 (B) shows an evaluation of the maximal distance of running. Exercise alone did not significantly improve the functional recovery of GA following VML injuries. VML+Foam+IGF+Exercise group demonstrated greater maximal running distance than VML and VML+exercise, indicating a synergistic effect of IGF foam with exercise on physiological recovery from VML. FIG. 24(C) shows an evaluation of GA functional recovery through maximum running distance measurements. FIG. 24(D) shows an evaluation of GA functional recovery after eight weeks of VML induction through in situ tetanus force measurements. Exercise alone did not significantly improve the muscle strength, while significantly higher average muscle force was observed in the Foam+IGF as compared to the VML group. The VML+Foam+IGF+Foam group showed significantly higher muscle strength than the VML. VML+exercise, VML+Foam+IGF groups, suggesting a synergetic effect of exercise on the Foam+IGF treatment following VML.

FIGS. 25A-25B show graphs illustrating the deformability of the foam biomaterial.

FIG. 26 shows a schematic of an exemplary foam fabrication and delivery process as disclosed herein.

FIG. 27 shows scanning electron microscope (SEM) images of exemplary Ge1MA foam at different scales.

FIGS. 28(A)-28(K) show the effect of exemplary Ge1MA foam releasing recombinant human proteoglycan 4 (rhPRG4) for immunoengineering. FIGS. 28(A)-28(F) demonstrate the effect of application of this system on macrophage polarization compared to topical administration of rhPRG4 at different concentrations. FIGS. 28(G)-28(K) demonstrate the release of different growth factors by macrophages as a result of exposure to the system.

FIG. 29 shows wound closure at different times for subjects having different patches over wounds.

FIG. 30 shows an image of a subject having an exemplary foam patch over a wound.

FIG. 31 shows the wound closure over 11 days as a result of exposure to topical rhPRG4, foam alone, and foam containing rhPRG4.

FIG. 32 is a block diagram of an exemplary delivery device as disclosed herein.

### DETAILED DESCRIPTION

The present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout. It is to be understood that this invention is not limited to the particular methodology and protocols described, as such may vary. It is also to be understood that the terminology used herein is for the purpose of describing particular embodiments only, and is not intended to limit the scope of the present invention.

Many modifications and other embodiments of the invention set forth herein will come to mind to one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

As used herein the singular forms “a,” “an,” and “the” include plural referents unless the context clearly dictates otherwise. For example, use of the term “a hydrogel” can refer to one or more of such hydrogels, and so forth.

All technical and scientific terms used herein have the same meaning as commonly understood to one of ordinary skill in the art to which this invention belongs unless clearly indicated otherwise.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint and independently of the other endpoint. Optionally, in some aspects, when values are approximated by use of the antecedent “about,” it is contemplated that values within up to 15%, up to 10%, up to 5%, or up to 1% (above or below) of the particularly stated value can be included within the scope of those aspects. Similarly, in some optional aspects, when values are approximated by use of the terms “substantially” or “generally,” it is contemplated that values within up to 15%, up to 10%, up to 5%, or up to 1% (above or below) of the particular value can be included within the scope of those aspects. When used with respect to an identified property or circumstance, “substantially” or “generally” can refer to a degree of deviation that is sufficiently small so as to not measurably detract from the identified property or circumstance, and the exact degree of deviation allowable may in some cases depend on the specific context.

As used herein, the terms “optional” or “optionally” mean that the subsequently described event or circumstance may or may not occur and that the description includes instances where said event or circumstance occurs and instances where it does not.

As used herein, the term “at least one of” is intended to be synonymous with “one or more of.” For example, “at least one of A, B, and C” explicitly includes only A, only B, only C, and combinations of each.

The word “or” as used herein means any one member of a particular list and, unless context dictates otherwise, can also include any combination of members of that list.

It is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation: and the number or type of aspects described in the specification.

The terms “subject” and “patient” are used interchangeably herein and refer to a human or animal to which the disclosed foam biomaterial is applied. In exemplary aspects, the human or animal can be a patient who is in need of treatment using the disclosed foam biomaterial. For example, a human or animal patient can be diagnosed with a condition in need of treatment using the disclosed foam biomaterial.

The following description supplies specific details in order to provide a thorough understanding. Nevertheless, the skilled artisan would understand that the apparatus, system, and associated methods of using the apparatus can be implemented and used without employing these specific details. Indeed, the apparatus, system, and associated methods can be placed into practice by modifying the illustrated apparatus, system, and associated methods and can be used in conjunction with any other apparatus and techniques conventionally used in the industry.

### INTRODUCTION

The present disclosure provides an advantageous system and method for formation of foam hydrogel bioinks for biomanufacturing. In particular, a simple, affordable and robust strategy may be utilized to generate foam hydrogel bioinks through a one-step mechanical agitation. Even more particularly, the generation of the foam hydrogel bioink may, in part, resemble formation of whipped cream used in pastries. As described herein and in the references disclosed herein and incorporated by reference in their entirety, the foam bioink may be formed by a one-step process that is robust and can be applied to most hydrogels and polymers. It may also enable the encapsulation of cells, molecules, proteins, biological factors, and other biological materials.

Upon crosslinking, a multiscale interconnected porous structure with pores ranging from less than about 1 to about several hundred micrometers is formed within the printed constructs. The effect of process parameters on the pore size distribution and mechanical and rheological properties of the bioinks is determined. The developed foam bioinks can be easily printed using conventional stationery and custom-built handheld bioprinters. It is demonstrated that the foam bioinks are biocompatible and enhance cellular growth and spreading. The subcutaneous implantation of scaffolds formed from foam bioinks shows their rapid integration, regeneration, and vascularization in comparison to their hydrogel counterparts. Foam-based bioink may emerge as a new class of inks for biomanufacturing in part because of their suitable rheological properties, multiscale porous structure, and desired biological properties.

In an exemplary embodiment, the present disclosure describes a porous bioink generated using a single-step foaming process. A hydrogel (e.g., Gelatin methacryloyl-Ge1MA) is mechanically agitated (i.e., homogenized by rapid stirring) to create a foam containing uniform and interconnected pore structures (up to about 80% porosity). Polyvinyl alcohol (PVA) may be added to increase foam stability after agitation. After foaming, cells are mixed with the foam bioink. Printing may be demonstrated using a 3D stationary and handheld bioprinter.

### Foam Biomaterial

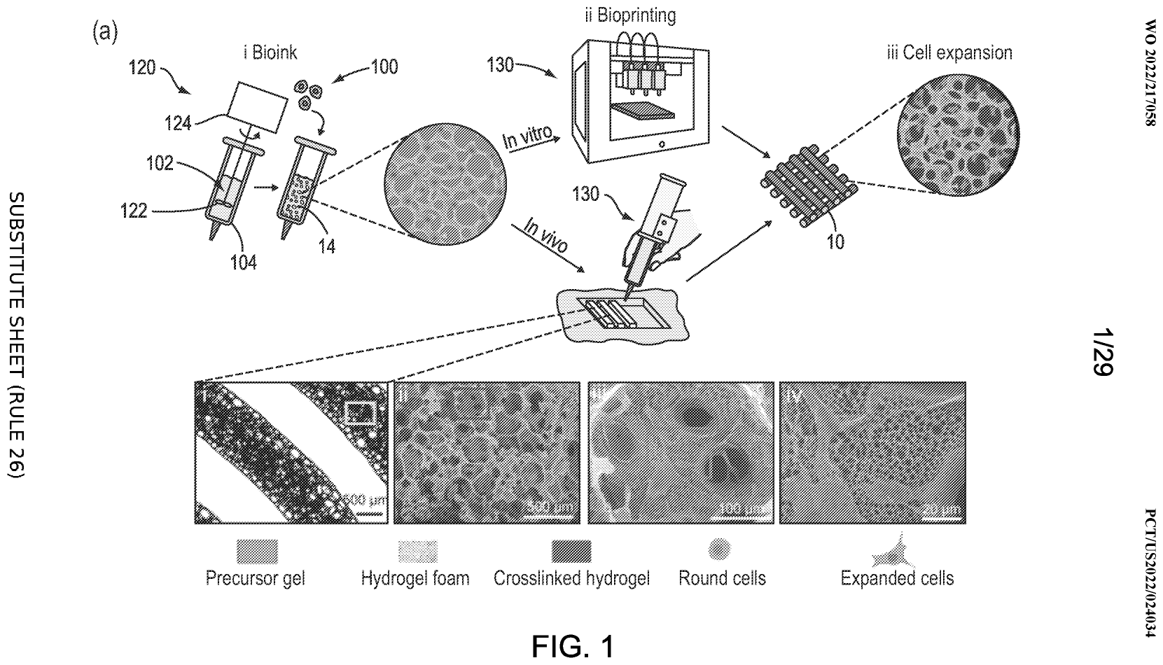

Disclosed herein, and with reference to FIG. 1, is a foam biomaterial 10 comprising a biomaterial having an interconnected pore structure. The foam biomaterial can have a porosity from about 10% to about 90%. In some aspects, the foam biomaterial 10 can be formed without the use of sacrificial materials. As can be understood, the structure of a foam biomaterial being formed without sacrificial materials can differ from the structure of a biomaterial having an internal structure that is formed using sacrificial materials.

In various optional aspects, the foam biomaterial can comprise at least one polymer, such as, for example, polycaprolactone, polylactic acid, at least one protein, (such as for example, collagen or gelatin), or a combination thereof. The polymer(s), protein(s), or combinations thereof can be functionalized with different chemicals and chemistries. The polymer(s), protein(s), or combinations thereof can be mixed with additives such as, for example, nanoparticles of any shape or microparticles of any shape, nanofibers, microfibers, or select chemicals. In some aspects, the additives can comprise metal, metal oxides, bioglasses, radiopaque agents, antibacterial compounds and agents, antibiotics, bioceramics, ceramics, oxygen-generating materials, proteins, vitamins, lipids, phospholipids, fatty acids, biological factors, polysaccharides, nucleic acids, growth factors, hydroxyapatite, calcium phosphate, dopamine-based material, carbon nanotubes, Quaternary ammonium compounds, polyhexamethylene biguanide (PHMB), methacry loyloxydodecylpyridinium bromide (MDPB), carbon nanotubes, graphene, graphene oxide, carbon derived materials, liquid crystals, peptides, chitosan, silver-based materials, platelet-rich plasma, blood-derived materials, and their combinations, etc. The concentrations of the additives can have a range from 0 to about 90% weight percent of the foam biomaterial.

In some aspects, the foam biomaterial can be crosslinked or solidified through physical, ionic, thermal, chemical, enzymatic, or photo crosslinking, or a combination thereof.

In some aspects, the foam biomaterial can be configured for depositing in or on a patient, and the foam biomaterial can adhere to surrounding tissue, a medical device, or other implanting materials (metallic, polymeric, ceramic, etc. implants) upon deposition and/or upon crosslinking.

In some aspects, the interconnected pore structure can support cellular infiltration, tissue remodeling, tissue regeneration, and/or tissue fidelity and stability.

In some aspects, the biomaterial or crosslinking mechanism can support or induce antimicrobial properties. For example, ultraviolet (UV) crosslinking (or other crosslinking methods) can have an antibacterial effect. In further aspects, the biomaterial ink can be an antibacterial or bacteria-resistant material.

In some aspects, the foam biomaterial can have a Young's modulus from 1 kPa to 100 MPa (e.g., from 40 kPa to 100 kPa).

In some aspects, the foam biomaterial can be capable of sustaining 50% strain for 20 cycles without breaking.

In some aspects, the pores of the foam biomaterial that are formed or generated by mixing can have a median diameter from about 20 microns to about 2,000 microns or from about 25 microns to about 1,000 microns or from about 50 microns to about 400 microns or from about 70 microns to about 90 microns. In these aspects, it is contemplated that other pores, such as micropores or nanopores, can inherently exist in the foam biomaterial (for example, due to the structural properties of the constituent materials of the solution, such as hydrogel). However, the pores generated by mixing can have larger dimensions (e.g., diameters) than the inherent pores. For example, the pores generated by mixing can be mesopores as further disclosed herein. Optionally, the inherent pores can have a median diameter from about 1 micron to about 15 microns or from about 2 microns to about 12 microns or from about 3 microns to about 10 microns. In further optional aspects, it is contemplated that each inherent pore can have a diameter of 15 microns or less or 10 microns or less, while each pore that is generated by mixing can have a diameter of 25 microns or more, 40 microns or more, or 50 microns or more. The pores generated by mixing can at least partially provide the interconnected pore structure disclosed herein that can advantageously permit cell diffusion. Optionally, the interconnected pore structure can comprise inherent pores, such as micropores or nanopores, that form connections between the larger pores (e.g., mesopores) that are formed or generated by mixing. FIGS. 1(b) and 27 show, from left to right, increasing magnifications of exemplary foam biomaterial, showing the structure of the pores.

In some aspects, the foam biomaterial can have a density from about 0.01 grams per milliliter to about 1.5 grams per milliliter (e.g., from about 0.25 grams per milliliter to about 0.50 grams per milliliter).

In some aspects, the foam biomaterial can have a complex viscosity from about 1 mPa·s to about 10 Pa·s that can be controlled by varying temperature, mechanical stress, or chemical composition.

### Method of Making Foam Biomaterial

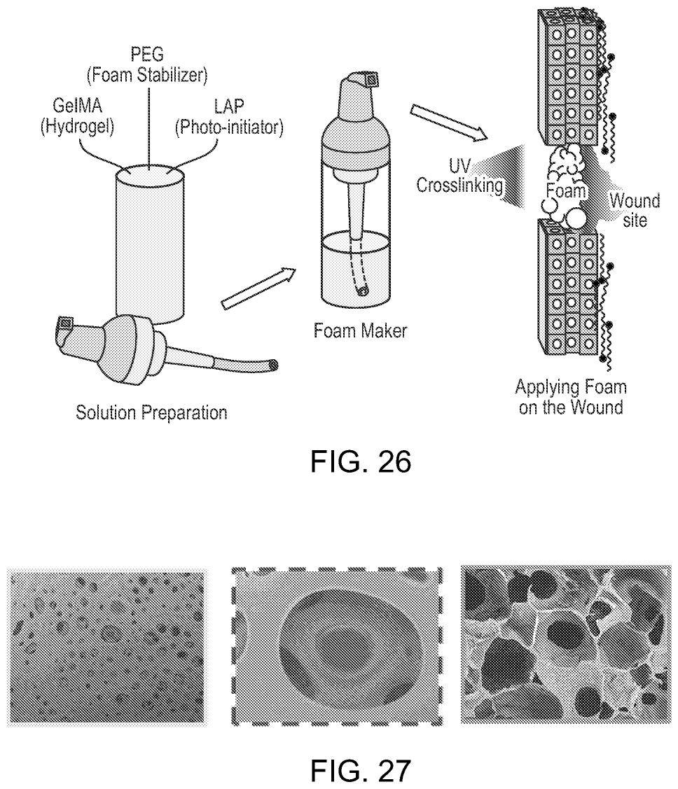

Referring to FIGS. 1 and 26, the foam biomaterial can be made by mechanically mixing a solution with a gas to generate a foam. In some aspects, the solution can be dynamically mixed, for example, by vortex mixing, shaking, acoustic actuation, or a combination thereof. In further aspects, the solution can be mixed with a gas via a static mixer. In still further aspects, bubbles (e.g., air or another gas) can be manually introduced into the solution. For example, a gas can be injected into the solution. The gas can optionally be injected into the solution under pressure.

The solution can comprise at least one biomaterial. For example, the solution can comprise at least one of: a polymer (e.g., a hydrogel), a protein, or a combination thereof. The solution can further comprise at least one additive, the at least one additive comprising nanoparticles, microparticles, an antibacterial compound, an antibiotic, a bioceramic, a ceramic, oxygen-generating material, at least one vitamin, at least one lipid, at least one phospholipid, at least one fatty acid, a biological factor, a polysaccharide, a nucleic acid, a growth factor, hydroxyapatite, calcium phosphate, dopamine-based material, carbon nanotubes, a Quaternary ammonium compound, polyhexamethylene biguanide (PHMB), methacryloyloxy dodecylpyridinium bromide (MDPB), graphene, graphene oxide, a carbon derived material, liquid crystals, peptides, chitosan, silver-based materials, platelet-rich plasma, a blood-derived material, or a combination thereof. In some optional aspects, the solution can comprise a hydrogel. Optionally, the hydrogel (e.g., optionally, can have a concentration from about 1% to about 30% w/v (e.g., optionally, Ge1MA) from about 5% to about 25%, or from about 10% to about 20% w/v).

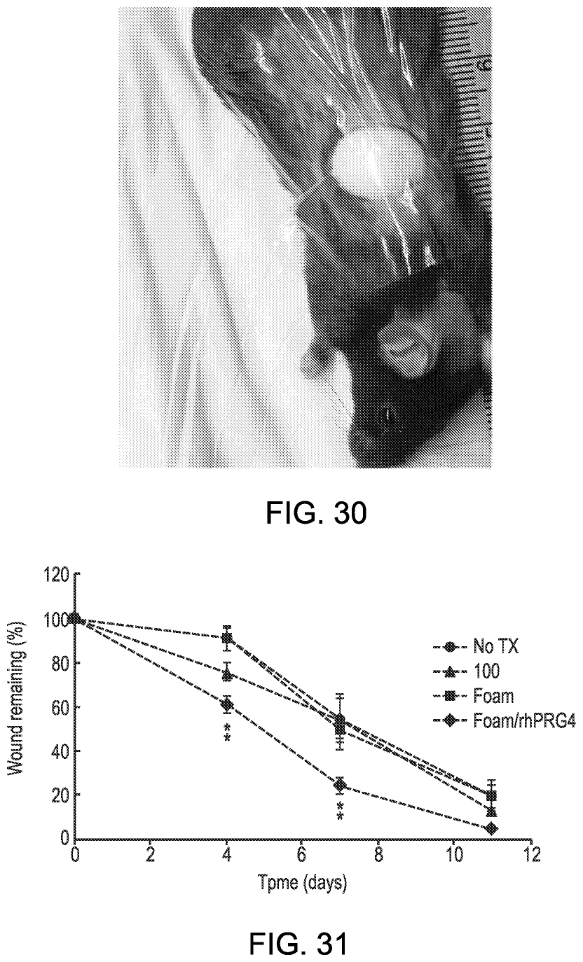

The foam can be deposited in a selected location. For example, the foam can be deposited in situ in or on a body of a patient. As used herein, the body of the patient can include a head, a torso, limbs, and all other parts of the patient. In some aspects, the foam can be deposited in or on a wound of a patient. For example, as shown in FIG. 30, it is contemplated that the foam can be deposited in the form of a patch on, within, and/or over a wound. In further aspects, the foam can be deposited in the body of the patient through a minimally invasive surgery method, such as, for example, laparoscopy, endoscopy, needle injection, intradermal delivery through needle arrays, or catheter-based delivery.

In further aspects, the foam can be deposited outside the body of the patient on a solid substrate or within a liquid substrate. The foam can be formed into a select shape. That is, the foam can be deposited to form a structure having a selected geometry. In some aspects, the foam can be deposited in the shape of the structure having the select geometry. In further aspects, the foam can be deposited and can then subsequently be formed into the shape of the structure having the select geometry.

The selected geometry can be set by crosslinking the foam. Upon forming the structure having the selected geometry, the structure can be placed in or on the body of the patient. For example, the structure having the selected geometry can be positioned in a cavity in the body.

In various aspects, the foam can be deposited with a 3D printer. The 3D printer can optionally be a handheld 3D printer, stationary 3D printer, robotic arm 3D printer, or catheter-based 3D printer.

In some aspects, the foam can be used as a supporting structure for secondary embedded biofabrication, biomedical device(s), or implantation material.

In some aspects, after depositing the foam in the selected location, the foam can be crosslinked to form the foam biomaterial having the interconnected pore structure. For example, the foam can be crosslinked with physical, mechanical, thermal, ionic, enzymatic, radiative crosslinking, or a combination thereof. In some aspects, the foam can be crosslinked either directly or indirectly through the skin of the body of the patient.

In some aspects, the foam biomaterial can adhere to surrounding tissue of the body of the patient. For example, the foam biomaterial can adhere to the surrounding tissue upon deposition or upon crosslinking. In further aspects, the foam biomaterial can adhere to implanting materials (metallic, polymeric, ceramic, etc. implants) upon deposition and/or upon crosslinking.

In some aspects, the foam biomaterial can be a space filler in the body of the patient.

In some aspects, the foam biomaterial can release or introduce a therapeutic agent encapsulated in its structure, for example, for enhanced regeneration, tissue maturation, tissue remodeling, cancer therapy, vaccine delivery, immune system modulation, etc.

In some aspects, the gas can be air. In further aspects, the gas can be selected for a particular application. For example, for a hypoxic area, the gas can comprise at least 50%, at least 60%, at least 70%, at least 80%, at least 90%, or all or substantially all oxygen. In further aspects, the gas can be controlled to adjust the cell culture. For example, the gas can comprise a mixture of oxygen, carbon dioxide, and nitrogen.

In some aspects, the physical, chemical, mechanical or rheological properties of the foam biomaterial can be controlled by adjusting or maintaining the temperature prior to or after deposition. For example, optionally, the foam can be held at a constant selected temperature prior to deposition.

In some aspects, depositing the foam and mechanically mixing the solution with the gas to generate the foam is performed in a single step. In further aspects, the solution can be mixed to form the foam, and the foam can subsequently be deposited. For example, optionally, the foam can be deposited into a deposition device prior to depositing the foam in the selected location. Said deposition device can be an in situ deposition device. For example, as illustrated in FIG. 1, the deposition device can be a handheld printer. In further aspects, the deposition device can be a robotic 3D printer.

In some aspects, cells can be added to the solution. Optionally, cells can be added to the foam. In further aspects, cells can be added to the solution prior to forming the foam. In yet further aspects, spheroids, organoids, tissue particles, etc. can be added to the solution prior to or after forming the foam.

In some aspects, the solution does not comprise a sacrificial material. In further aspects, the solution does not comprise a porogen. In further aspects, the foam is not generated through lyophilizing. In further aspects, the foam is not generated by casting a solution on sacrificial particles such as salt particles, paraffin beads, and/or ice crystals.

In some aspects, the solution can comprise an emulsifier. In further aspects, the solution can comprise a surfactant. The surfactant can stabilize the foam. In further aspects, the solution can comprise one or more foam boosters. Exemplary surfactants can include polyvinyl alcohol and/or sucrose. Exemplary emulsifiers can include lecithin and/or sugar. Exemplary foam boosters can include sodium lauryl sulfate.

The solution can be mixed to provide a tailored porosity. The porosity can be controlled by the composition of the solution, the mixing speed, and the mixing time. Accordingly, the solution can be mechanically agitated for a predetermined time and at a predetermined speed to provide the tailored porosity.

In further aspects, the porosity can be spatially controlled. Thus, porostity need not be constant throughout the foam biomaterial. For example, a spatial gradient can be generated. Said spatial gradient can optionally be formed with a static mixer (e.g., a Christmas tree mixer). In exemplary aspects, the porosity can gradually change moving in at least one direction along the foam biomaterial.

Optionally, the solution can be mixed in a syringe barrel to form the foam. For example, a stirring element can be inserted into the syringe barrel. After forming the foam, a plunger can be inserted in the syringe barrel.

The foam can have an interconnected pore structure after deposition. For example, upon crosslinking or otherwise curing, the foam can form and maintain the interconnected pore structure. In this way, cells can grow within the interconnected pore structure.

### System for Making Foam Biomaterial

Referring to FIG. 1, a system 100 for making a foam biomaterial 10 as disclosed herein can comprise a solution 102 in a vessel 104, the solution comprising a biomaterial. The system 100 can further comprise a mixer 120 that is configured to combine the solution with a gas to generate a foam 14.

In some optional aspects, the mixer 120 can comprise a stirring element 122. The mixer can further comprise a motor 124 that is coupled to the stirring element 122. The motor 124 can be configured to cause the stirring element to rotate. The stirring element 122 can be configured to be inserted into the vessel to mix the solution with air or surrounding gas.

In further aspects, the mixer 120 can comprise a vortex mixer, a shaker, an acoustic mixer, a bubbler, a static mixer, a pressure induced foaming system, or combinations thereof.

In further aspects, the mixer can be a static mixer.

In some optional aspects, the vessel 104 can be a syringe barrel. Optionally, at least a portion of the mixer can be positioned within the syringe barrel. For example, the stirring element 122 can be inserted into the vessel 104.

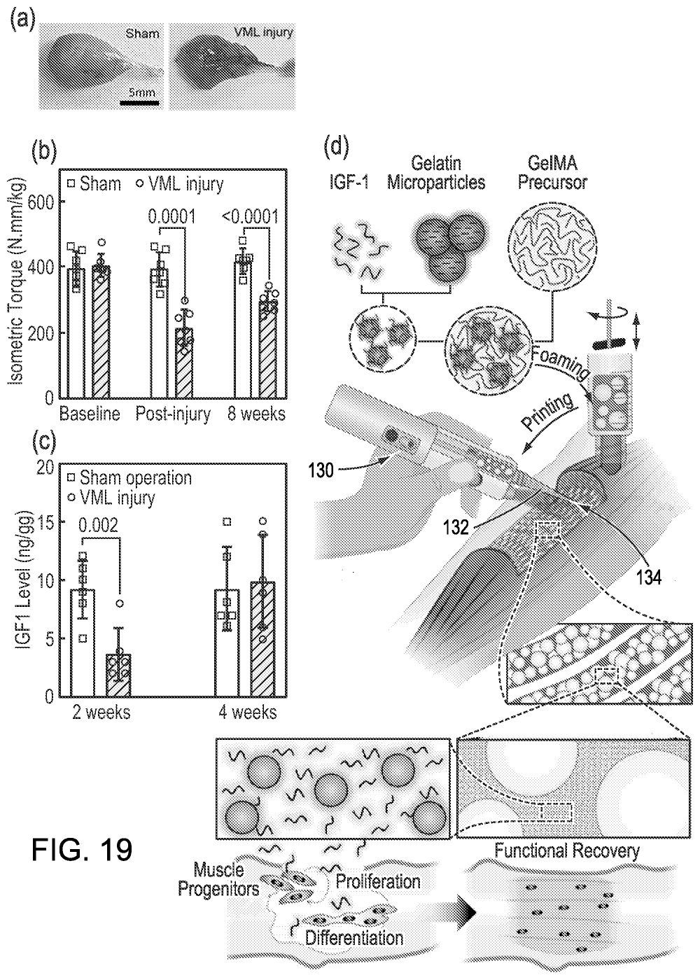

Referring to FIGS. 1 and 19, the system 100 can further comprise a deposition device 130. The deposition device 130 can comprise a nozzle 132 defining an outlet 134. An actuator 136 can be configured to extrude the foam through the outlet of the nozzle. The actuator 136 can be, for example, an electromechanical linear actuator that drives the foam through the outlet 134.

Optionally, the deposition device 130 can be configured to be handheld. In this way, the deposition device 130 can enable in situ deposition, as further disclosed herein.

In further aspects, the deposition device 130 can comprise a robotic arm that is configured to position the nozzle.

In still further aspects, the deposition device 130 can comprise one of an endoscopy, a needle injector, an intradermal needle array, or a catheter. In this way, the foam can be delivered to various parts of the body for various different applications.

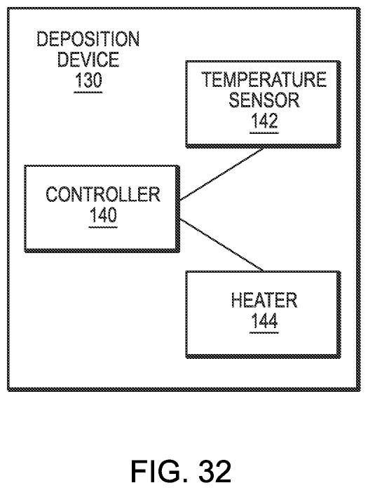

Referring to FIG. 32, in some optional aspects, the deposition device 130 can comprise a controller 140, a temperature sensor 142 in communication with the controller, and a heater 144 in communication with the controller. The heater 144 can be configured to control the temperature of the foam. For example, the heater 144, in cooperation with the controller 140 and temperature sensor 142, can be configured to maintain the foam at or substantially at a selected temperature.

### Example 1

Referring to FIG. 1, in an exemplary embodiment, the present disclosure describes a porous bioink generated using a simple single-step foaming process. A hydrogel (e.g., Gelatin methacryloyl—Ge1MA) is mechanically agitated (i.e., homogenized by rapid stirring) to create a foam containing uniform and interconnected pore structures (up to about 80% porosity). PVA may be added to increase foam stability after agitation. After foaming, cells are mixed with the foam bioink. Printing may be demonstrated using a 3D stationary and handheld bioprinter.

Three different concentrations of Ge1MA foam were characterized (i.e., physical/mechanical properties) and compared to the original Ge1MA. Biocompatibility foam bioink assessed—rat model. To investigate the biocompatibility of foam constructs and their ability to support regeneration in vivo, both foam and gel scaffolds were implanted subcutaneously in rats and monitored for 4 weeks.

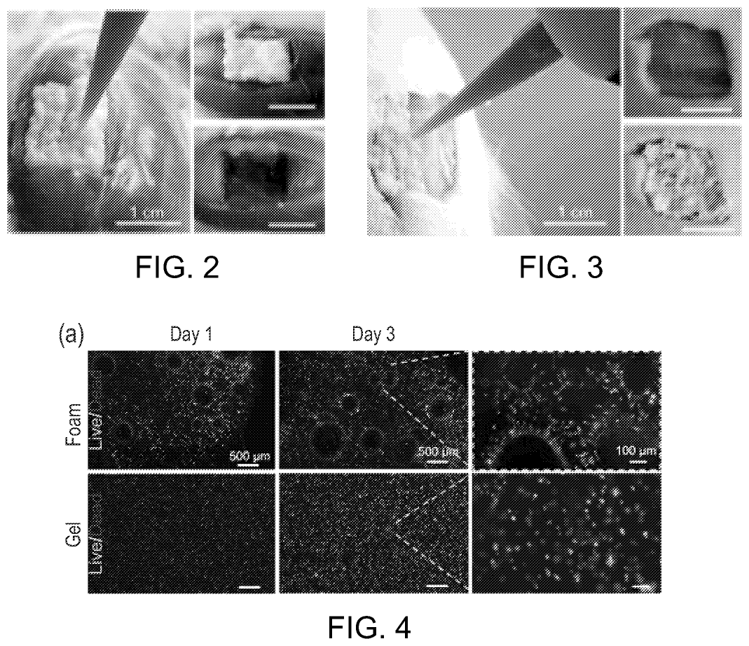

FIG. 2 shows ex vivo printing of the foam bioinks into induced VML and FIG. 3 shows wound injury models to show the feasibility of foam in vivo printing and its adhesion to the surrounding tissue.

In vitro and in vivo biocompatibility assessment of the foam and normal gel scaffolds was performed.

FIG. 4 shows Live/Dead staining demonstrating the viability of the cells (live: green, dead: red) encapsulated cells in the foam and gel scaffolds at day 1 and 3 post printing.

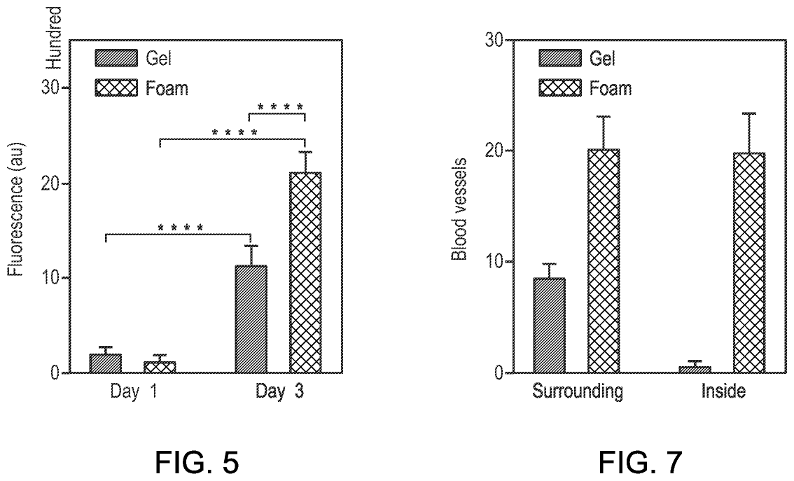

FIG. 5 shows in vitro results of the PrestoBlue assay, indicating the cells proliferation profile cultured for 3 days in foam and gel scaffolds.

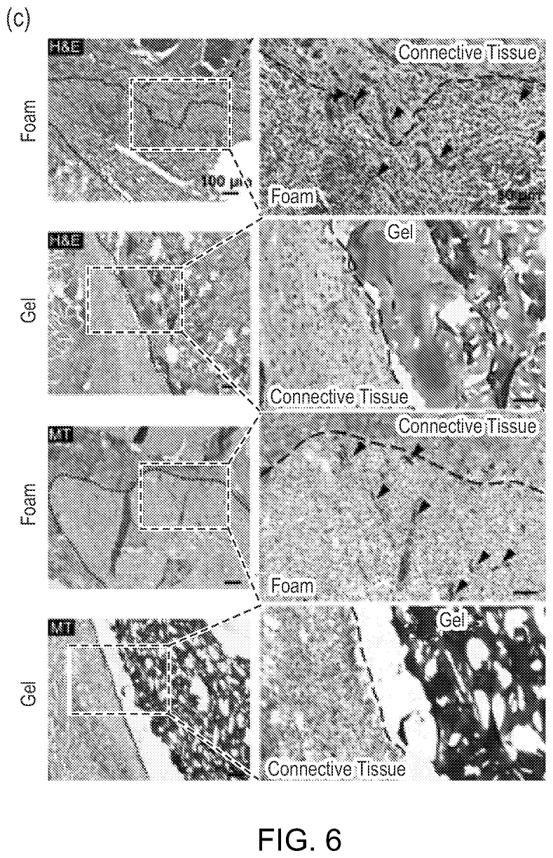

FIG. 6 illustrates subcutaneous implants in vivo. Both foam and gel scaffolds were implanted subcutaneously in rats and monitored for 4 weeks. Hematoxylin and eosin (H&E) and Masson's trichrome (MT) staining of the scaffolds-tissue interface one-week post implantation is shown. Black arrowheads pointing to neovessels formed in the foam scaffold, demonstrating the high level of foam-tissue integration.

FIG. 7 shows the quantitative evaluation of in vivo tissue vascularization in surrounding tissue and inside the implanted scaffold one-week post implantation.

Commercial applications may include, but are not limited to:
Treatment of musculoskeletal injuries
Biomedical applications—e.g., tissue engineering/regenerative medicine
Wound care
Anti-adhesion coating
Food manufacturing

In another embodiment, the foam-based bioink can be utilized with hand applicators (e.g., hand pumps, spray applicators, aerosol cans) to deliver the foam-based bioink.

### Example 2

Tissue engineering and regenerative medicine have generated substantial promise toward developing effective treatments for various diseases and medical complications [1]. Engineering 3D cell scaffolds, as the main component in tissue engineering, is therefore of substantial importance, directly affecting the treatment outcomes [2-4]. The fabricated scaffolds should offer proper biological and physical properties supporting cellular activity including migration, growth, differentiation, and maturation [5,6].

Three dimensional (3D) bioprinting technologies have received significant attention due to their ability to fabricate complex constructs by precise deposition of bioinks to form 3D constructs [7,8]. Among various bioprinting approaches, extrusion-based bioprinting has attracted more attention due to its compatibility with a wide range of bioink viscosities, decent reliability, and capability of large-scale scaffold fabrication with clinically relevant dimensions [9,10]. Extrusion-based bioprinters also allow the fabrication of multi-materials scaffolds [7]. However, the quality of printing and the system reproducibility heavily depends on the bioink characteristics [11]. In addition to optimal printability, the bioink should offer good cell permissibility. Various biomaterials have been implemented for the 3D bioprinting of tissue engineering scaffolds. Among them, hydrogels have been broadly applied as bioink due to their similarity to the natural extracellular matrix (ECM) and tunability of their properties [12,13]. Hydrogels are networks of hydrophilic polymer chains with a nano- to micro-scale porous structure [14,15]. The porous structure allows gas and small molecules to diffuse throughout the network providing a nurturing environment for encapsulated cells [16,17].

However, by increasing the bulk size of the hydrogel scaffold, the diffusion capability is severely diminished which results in undesired cell death within the depth of the construct [18]. Slow cell infiltration further limits the early vascularization and innervation of the implanted scaffold and therefore impairs encapsulated cells' viability and functionality [19,20]. Incorporation of micro to millimeter-scale channels within the scaffold through multi-material extrusion 3D bioprinting is a possible resolution [7]. However, this porosity could negatively influence the mechanical properties, fidelity, and structural stability of the final scaffold. Therefore, a high concentration of the hydrogel bioinks is required to stabilize the printed structure, which in turn limits cell spreading, migration, and tissue integration [21-24]. While prevascularization and preinnervation strategies can also be implemented [25], these strategies make the 3D bioprinting and subsequent tissue implantation highly complex.

An alternative approach to overcome this issue is introducing interconnected multiscale pores within the hydrogel network to enable efficient mass transport, allow rapid cell infiltration, offer more cell anchoring surfaces, and therefore facilitate cell expansion, proliferation, and tissue regeneration [19]. In contrast to conventional hydrogels, it has been previously shown that macroporous hydrogels enhance cellular ingrowth [26]. Several techniques developed to generate macroporous hydrogels such as freeze-drying [27], gas foaming[28], and agent leaching strategies [29,30]. However, most of these approaches are not compatible with 3D bioprinting as most of the processes involve the use of toxic materials that can negatively impact the encapsulated biological materials [31]. Alternatively, microgel-laden bioinks and aqueous-biphasic systems are reported which allow cell incorporation during macroporous bioink preparation: however, these approaches demand multiple washing steps of the hydrogel structures to remove the sacrificial polymer and make them impracticable for in vivo printing applications [32-34]. Recently, an approach has been developed based on the introduction of air bubbles into the hydrogel through a pulling/pushing procedure of the solution using a syringe. Although the formed bubbles enhance cell viability in the core of the hydrogel, there was no control over the bubble size and distribution [35].

In this example, porous bioinks were developed through a foaming process that did not involve any toxic materials or multistep processes. The hydrogel can be mechanically agitated by simple stirring at relatively high rates (e.g., optionally, 5,000-20,000 rpm) to generate a foam with a uniform and interconnected pore structure, resulting in up to 80% porosity. The foam bioink which acted as a shear-thinning material was then used as a bioink for 3D bioprinting of scaffolds (FIG. 1(a)). Gelatin methacryloyl (Ge1MA) was used as the hydrogel solution for the foam preparation. Ge1MA was selected due to its promise in various tissue engineering applications [14,36]. The physical and mechanical properties of the foam generated from three different concentrations of Ge1MA were characterized and compared to the original Ge1MA. The printability of the foam bioink was investigated with a 3D bioprinter as well as a custom-built handheld bioprinter [37]. The biocompatibility of the developed foam bioink was further assessed in vitro and in vivo through subcutaneous implantation in a rat model.

### Results

Bioink preparation and physical characterization of foam scaffolds

A Ge1MA solution was agitated using a homogenizer at high speed to form a foam. To increase the stability of the foams, 1% (w/v) polyvinyl alcohol (PVA), a synthetic biocompatible polymer, was added to the solution as a surfactant [38,39]. FIG. 1 demonstrates an overview of the preparation process of cell-laden foam bioinks for 3D bioprinting of multiscale porous constructs. After foaming, cells can be mixed with the foam bioink and printed using a 3D stationary or a handheld bioprinter. FIG. 1(b) illustrates the hierarchically distributed macro- to micro-pores within the 3D printed scaffolds. A bright-field micrograph (FIG. 1(b)(i)) shows the macroporous (≥500 μm) structure formed through the extrusion 3D bioprinting process. FIG. 1(b)(ii, iii) shows scanning Electron Microscopy (SEM) images from 3D printed filaments (exhibit the interconnected mesoscale pores (50-400 μm) generated during the foaming process. FIG. 1(b)(iv) shows the microporous structure of native Ge1MA hydrogel formed upon polymeric chains crosslinking [15]. As disclosed herein, the interconnected pore structure can be formed by the mesoscale pores generated by mixing the solution with gas.

FIG. 1 illustrates the concept of engineering foam bioinks for 3D bioprinting of scaffolds. FIG. 1(a) shows a schematic illustration of 3D bioprinting multiscale porous structure using foam-based bioinks. The preparation process of the cell-laden foam bioink through hydrogel precursor stirring followed by cell addition: (ii) 3D bioprinting of the foam bioink in vitro using a stationary bioprinter or in vivo utilizing a handheld bioprinter: (iii) Multiscale porous structure of the bioprinted foam, facilitating cell expansion in the printed constructs. FIG. 1(b) shows micrographs from hierarchical macro- to micro-scale pores within the 3D printed scaffolds: FIG. 1(b)(i) shows a representative bright-field microscopy image from macroporous 3D printed structure: FIG. 1(b)(ii-iv) show representative SEM images from printed foam bioink filaments, FIG. 1(b)(ii, iii) interconnected mesopores structure formed by a foaming process, FIG. 1(b)(iv) microporous Ge1MA structure formed after crosslinking of the polymer chains.

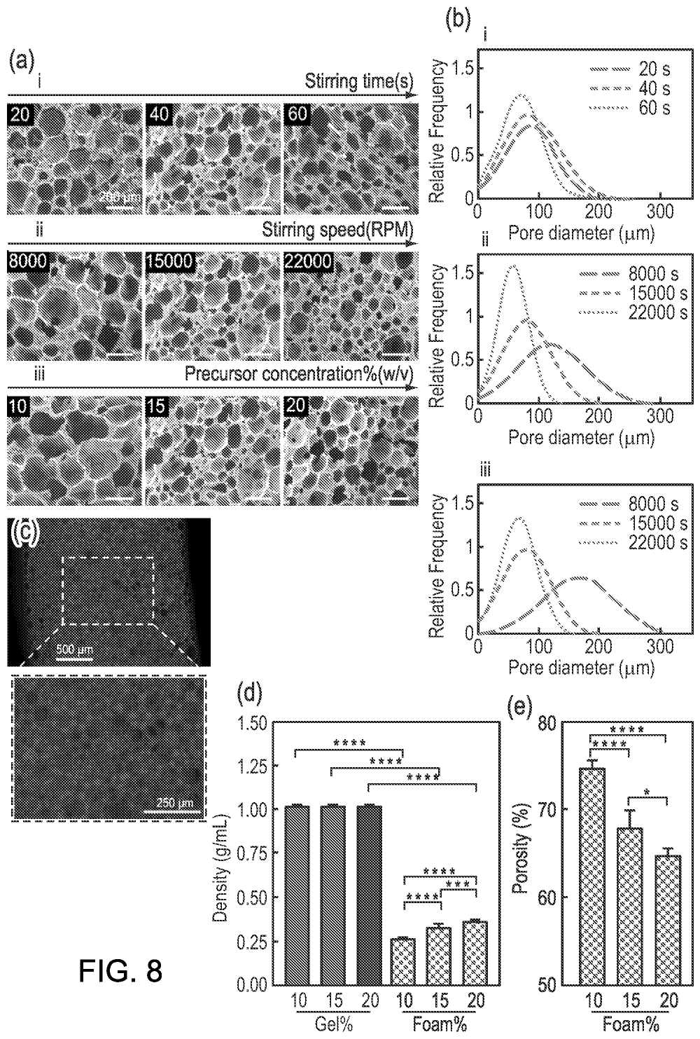

The visual and quantitative representation of the pore size distribution inside the foam bioink are shown in FIG. 8(a) and FIG. 8(b), respectively. As expected, the pore size was decreased with increasing the stirring time, stirring speed, and the concentration of the hydrogel, while the effects of stirring speed (FIG. 8(a-ii), 8(b-ii) and hydrogel concentration (FIG. 8(a-iii), FIG. 8(b-iii) were more significant. This fact demonstrates the shear-dependent mechanism of microbubble formation in the foaming process. During the foaming process, a large amount of air is first introduced into the liquid through stirring at the liquid-air interface. Subsequently, the captured bubbles inside the liquid are split into smaller bubbles because of shear stress dominating the surface tension of the bubbles. Increasing the stirring speed and hydrogel viscosity directly increases the shear stress applied to the bubbles during the foaming process and therefore reduces the bubble size (FIG. 8(a-ii), FIG. 8(a-iii) and FIG. 8(b-ii), FIG. 8(b-iii)). While most bubbles quickly break up upon experiencing shear stress caused by mechanical stirring, the longer duration of shear force exertion can result in further splitting of bubbles. Therefore, the time of the stirring is expected to affect the dispersity of the size distribution, which is correlated to the width of normal distribution graphs shown in FIG. 8(b). The results demonstrated that while increasing the stirring time can decrease the size dispersity, even a 20 sec stirring generates an almost homogeneous structure when a 15,000 rpm was used for foaming of 15% Ge1MA (FIG. 8(a-i) and FIG. 8(b-i)). The significant change in pore size distribution by varying the stirring speed and hydrogel concentration further shows that the required time for obtaining a homogeneous foam structure is shear-dependent. Higher shear stress rates due to the faster stirring speed (FIG. 8(a-ii), FIG. 8(b-ii)) and hydrogel concentration (FIG. 8(a-iii), FIG. 8(b-iii)) could decrease the required time for the formation of foam with monodisperse pore size.

Furthermore, the pores preservation in foam bioink during the extrusion process of printing and the scaffold stability in physiological conditions were assessed. Rhodamine B-loaded Ge1MA foam was printed and the printed structures were examined using a fluorescent microscope, after incubation in Dulbecco's phosphate-buffered saline (DPBS) solution at 37° C. (FIG. 8(c)). The micrographs showed that the pores were stable in filaments upon printing and the pore size distribution was homogenous. Furthermore, it was observed that the porous scaffolds were stable after incubation at 37° C. A higher magnification micrograph is shown in FIG. 8(c-i) indicates the interconnectivity of the pores.

The formation of a mesoporous structure within a biofabricated scaffold is an important factor since it offers cell ingrowth spaces without the need for scaffold degradation. The density and porosity of the foam bioinks were further evaluated and compared with the gel precursor. As expected, the density of foam scaffolds was significantly lower than their Ge1MA counterparts, which is associated with the integration of air bubbles into the scaffolds during the foaming process (FIG. 8(d)). The calculated porosity of the foam bioinks was around 75%, 68%, and 65%, respectively for the foams generated from 10%, 15%, and 20% Ge1MA (w/v) (FIG. 8(E)). This clearly demonstrates the significant increase in free space available for cellular ingrowth in the foam bioink.

FIG. 8 shows a characterization of porosity in printed foam constructs. FIG. 8(a) shows tunability of the pore size and distribution within the foam bioink through manipulation of (i) stirring time, (ii) stirring speed (RPM), and (iii) precursor hydrogel concentration. FIG. 8(b) shows the quantitative representation of pore size distribution corresponding to groups shown in FIG. 8(a) (n=3). FIG. 8(c) shows the structural stability of printed foam filaments after 1 h immersion in DPBS at 37° C. Fluorescent images captured from Rhodamine B loaded foam filaments demonstrates the stability of the interconnected pores after extrusion and incubation in physiological condition. FIG. 8(d) shows a comparison between the density of gel and foam bioinks (n=4). FIG. 8(e) shows a calculated porosity in foam bioinks made with different Ge1MA concentrations (n=4).

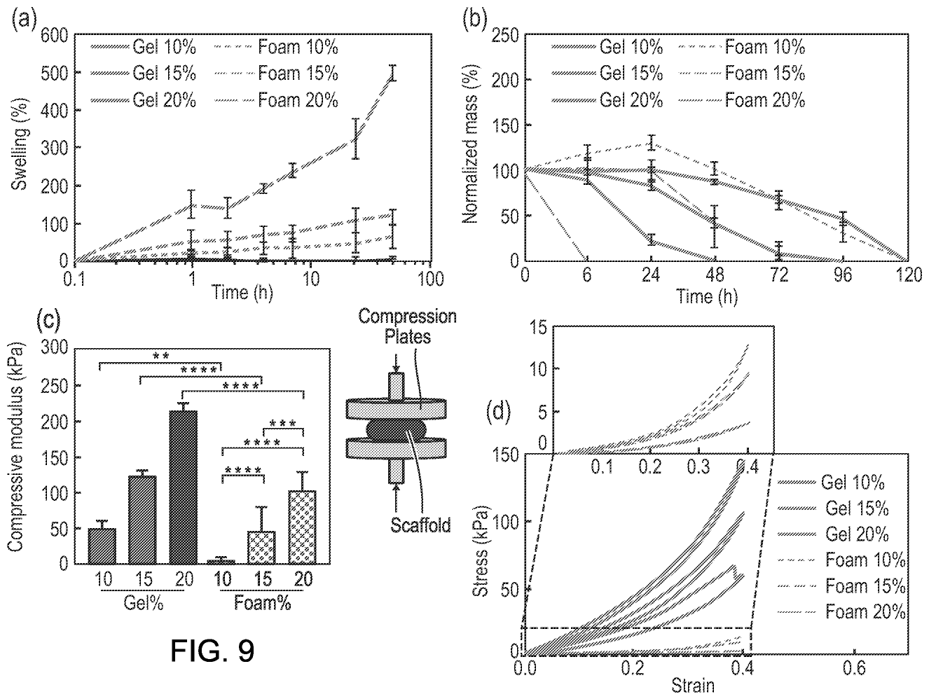

To evaluate the swelling behavior of the printed foam scaffolds, samples were further incubated in DPBS for a longer time. The results indicated a high swelling ratio in foam scaffolds, especially in lower hydrogel concentrations. The swelling ratio increased to 499+20% after 48 h in foam samples made of 10% (w/v) Ge1MA which was ˜1% in gel scaffolds with the same concentration (FIG. 9(a)). The swelling ratio in foam scaffolds made of 15% and 20% (w/v) Ge1MA, were 121+26% and 66+31%, respectively. The results suggest better structural stability of foams made of higher Ge1MA concentrations.

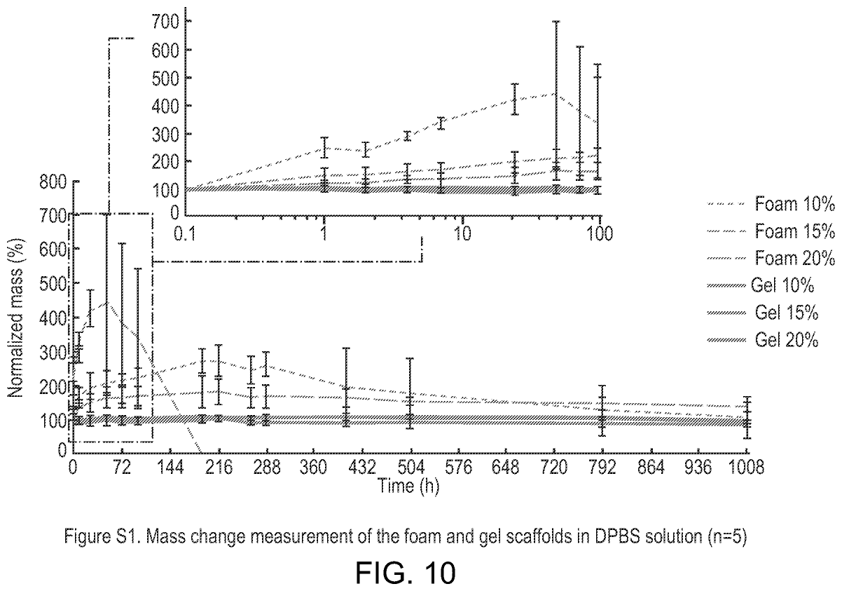

The mass change of the scaffolds was also measured over time. Two conditions were considered for this experiment: (i) incubation in DPBS and (ii) incubation in DPBS containing collagenase type I to mimic expedited degradation of gelatin-based materials in vivo. Samples were incubated at 37° C., and weighted at each time point. The results demonstrate that in the presence of collagenase, the degradation of foam scaffold made of 10% (w/v) Ge1MA is much faster than its counterpart gel scaffold (FIG. 9(b)). The mass change ratio in the foam samples made of 15% (w/v) and 20% (w/v) Ge1MA initially increased slightly (31+8% after 24 h in foam samples with 20% Ge1MA): this suggests that swelling dominates the degradation at earlier time points. However, the samples' mass started to decrease after 24 h and a comparable mass change between the foam samples and gel scaffolds was observed at later time points. Both foam and hydrogel bioinks containing 15% (w/v) Ge1MA and 20% (w/v) Ge1MA entirely degraded after 96 h and 120 h, respectively. It should be noted that this experiment demonstrated an expedited degradation, and the results can be used to predict the trend of the mass loss and the degradation phenomenon in vivo. Perhaps the rate of degradation in vivo is expected to be slower. The mass changes of the samples were also measured in DPBS without collagenase to assess their long-term stability. The results indicated that foam scaffolds made from 10% (w/v) Ge1MA degraded after 8 days, suggesting limited stability (FIG. 10). Additionally, the mass of the foam samples made from 15% (w/v) and 20% (w/v) Ge1MA increased up to 9 days and then started to decrease (FIG. 10). Overall, both 15% (w/v) and 20% (w/v) Ge1MA foam samples were stable for at least 42 days. These results suggested that the foam structures made of 10% (w/v) Ge1MA solution were not stable enough for most tissue engineering applications.

### Characterization of the Mechanical Properties of the Foam Scaffolds

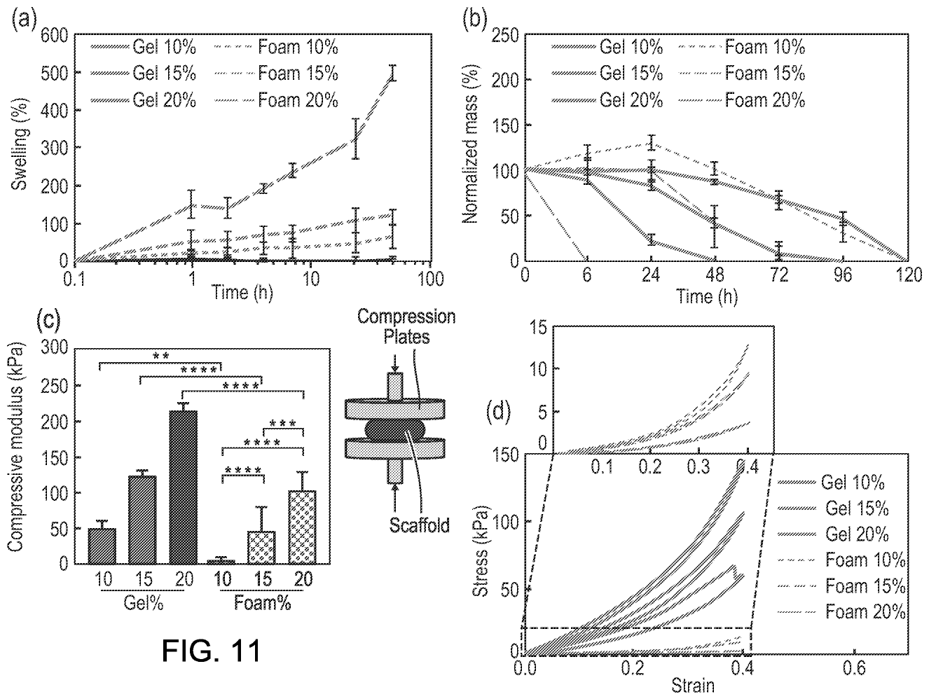

Although physical modifications of the bioink to increase its porosity can improve the cell permissibility and mass transport through the scaffold, they might have negative effects on the mechanical stability of the printed constructs. Therefore, the mechanical properties of the scaffolds fabricated from foam bioinks with different Ge1MA concentrations were assessed and compared with gel samples (FIG. 11(c,d)). The results of compression tests indicated that the average Young's modulus in foam bioinks increased from 4+4 kPa to 46+33 kPa, and 102+26 kPa by changing the Ge1MA concentration from 10% to 15%, and 20% (w/v), respectively. Overall, foam scaffolds showed lower Young's modulus in comparison to gel samples. However, the compressive moduli of foam bioinks were in the range of compressive modulus of soft tissues [40]. While a minimum mechanical stiffness is important for the fidelity and stability of the printed constructs, usually high mechanical properties are not favorable for 3D cell scaffolds applied in soft tissue regeneration due to their limited cell permissibility.

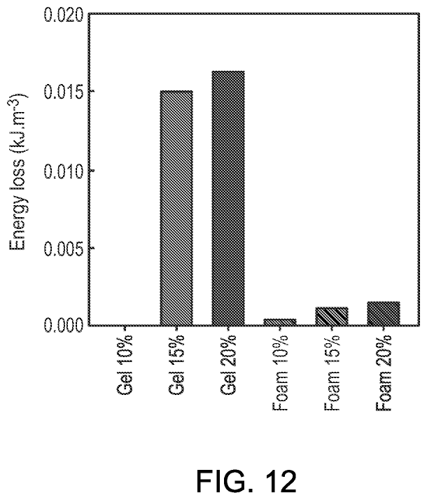

Furthermore, cyclic compression tests were performed to evaluate the endurance of the scaffold against repetitive loads applied to the implanted scaffolds during body motion. Cyclic compression tests were performed at 50% strain up to 20 cycles. Interestingly, all of the foam samples could sustain the cyclic loading, while the 10% (w/v) gel scaffolds broke at the first cycle. FIG. 11(d) demonstrates the stress-strain plots obtained during the 10^{th} loading cycle. Similar to normal compression tests, decreasing in stiffness of the scaffold was observed by reducing the Ge1MA concentration. The energy loss calculation showed the same trend in the mechanical properties (FIG. 12). The results indicated that although 10% (w/v) Ge1MA foams have more ductility than 10% (w/v) gel scaffolds, its low Young's modulus makes it impractical for bioprinting of tissue engineering constructs.

FIG. 11 shows the physical and mechanical characterization of the foam scaffolds in comparison to gel samples. FIG. 11(a) shows the swelling ratio of the foam and gel scaffolds with different concentrations shows higher swelling of foam scaffolds (n=5). FIG. 11(b) shows the mass change measurement of the foam and gel scaffolds in collagenase type I (20 μg/mL in DPBS) solution to mimic an enzymatic degradation of gelatin-based materials in vivo (n=5). FIG. 11(c) shows the compressive moduli of disk samples fabricated from different foam and gel bioinks (n≥5) and FIG. 11(d) shows their behavior under cyclic compression. The graph shows the material behavior at the 10^{th} loading cycle 10 (except Ge1MA 10% which broke at its first cycle). The inset shows the magnified plots obtained from the loading of foam scaffolds (n=2).

### Characterization of the Rheological Properties and the Printability of the Foam Bioinks

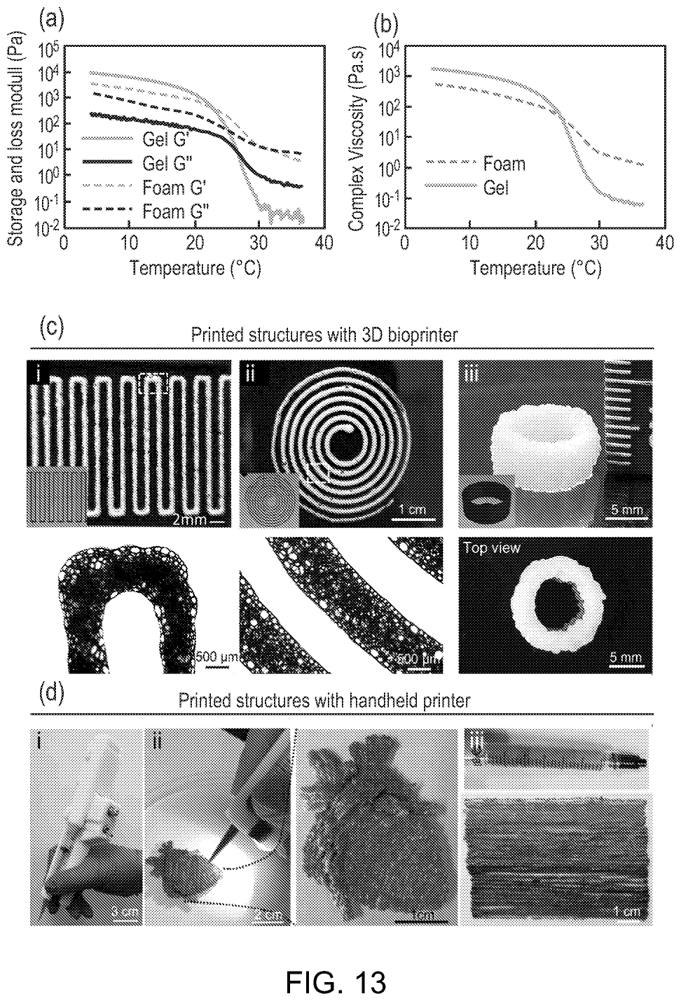

The rheological properties of the bioinks significantly affect their printability. While Ge1MA is a promising material for 3D cell scaffolds, its low viscosity and slow photocrosslinking limited its application as a bioink in bioprinting [41]. A strategy to resolve this problem is partial crosslinking of the hydrogel precursor through decreased temperature [41]. However, the rapid sol-gel transition of Ge1MA in decreased temperatures, causing complete thermal crosslinking and therefore increased mechanical properties during printing, limits the printability window and makes the Ge1MA bioprinting unreliable. FIG. 13(a) shows the storage and loss moduli of the Ge1MA, in comparison to foam bioink with similar Ge1MA concentration. The storage modulus of 15% Ge1MA bioink experienced around a 1000 fold increase during its sol-gel transition between 30° C.-25° C., making the bioprinting of partially gelled bioink highly unstable. On the other hand, the 15% Ge1MA foam bioink only experienced around 14 fold increase during the sol-gel transition in the same temperature range, which makes it much easier to print using the partial gelation strategy.

Increasing the Ge1MA concentration to enhance its viscosity is another possible solution to facilitate Ge1MA 3D printing, however, it causes poor cellular activity upon photocrosslinking as a result of high mechanical properties [24]. FIG. 13(b) compares the viscosity of the gel and foam bioinks with similar concentrations (15%). Interestingly, the viscosity of the foam precursor is in the order of 2 Pa·s, which is 30 times more than the Ge1MA precursor (˜0.07 Pa·s), enabling the facile 3D bioprinting of the foam bioink. Furthermore, lower mechanical properties of the foam along with the presence of intrinsic pores, described previously, improve the cellular activity within this bioink compared to the Ge1MA, reducing the challenge of cell permissibility in concentrated Ge1MA bioinks used for enhanced printability.

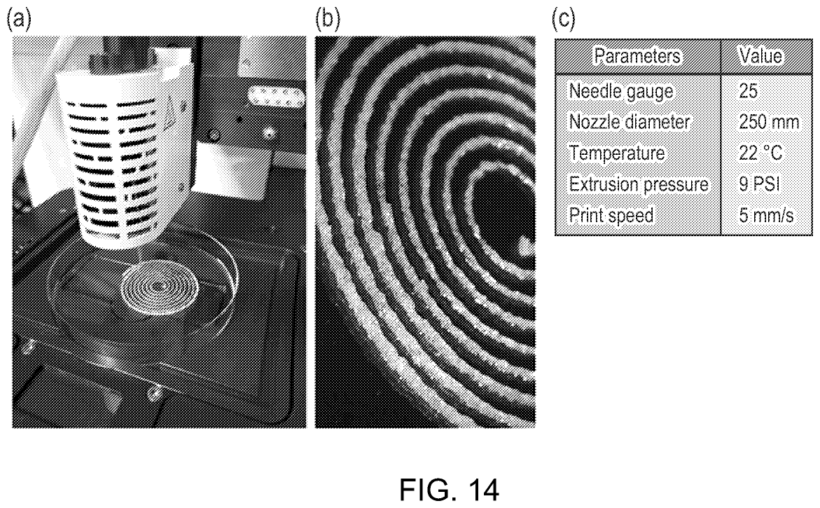

The printability of the foam bioink was validated by assessing the quality of printing 3D structures using a commercial extrusion-based 3D bioprinter (FIG. 13(c), FIG. 14). As can be seen from the bright-field microscopic images of the 3D printed structures, the printed foam filaments have a consistent thickness and porosity distribution. This implies that the incubation period inside the 3D printer and extrusion process even from a relatively narrow tip (250 μm) does not affect the foam and deposited filament structures. In order to assess the feasibility of foam bioink application in the printing of 3D structures, a multi-layer (for example, 16 layers) hollow cylinder, with 9 mm inner diameter, 1 mm thickness, and 5 mm height, was constructed. As shown in FIG. 13(c-iii), the foam bioink allows the fabrication of a 3D structure with good fidelity without requiring any supporting material or structures. Increased viscosity of the foam along with rapid sol-gel thermal transition upon printing offered facile 3D printing of the constructs with high structural stability, even before photocrosslinking. The printing parameters including temperatures, pressures, and speeds were optimized for enhanced printability of the foam bioink with continuous filament structure, preventing any “over-flowing” or “dash-printed” patterns (FIG. 14). The best printing quality was obtained at 9 PSI extrusion pressure, 5 mm·sec⁻¹ printing speed, printing in ambient temperature (22° C.), and extrusion through a 25-gauge tapered plastic tip. Representative micrographs of three different printed architectures are demonstrated in FIG. 13(c-i-iii).

As direct in vivo printing of scaffolds has drawn significant attention recently, the suitability of foam bioinks for in vivo printing applications was also explored using a partially automated custom-designed handheld printer (FIG. 13(d-i)). The flow rate was set to 5 μl/s, and a 22G tapered plastic tip was used as the nozzle. FIG. 14(d-ii) illustrates a heart shape structure printed with two different colored foam bioinks to show the quality of the printing using a handheld bioprinter.

In many tissue engineering applications, graded structures with gradient factors are needed to enable the reconstruction of different tissue interfaces. However, a multi-head bioprinter with the capability of multi-material bioprinting is not always accessible. If the printer does not offer such capability the bioprinting of graded structures is extremely challenging as the inks carrying different cells or active molecules cannot be kept distinctly in a single container. However, in the case of foam inks, the mixing of the different inks can be minimal: therefore, graded structures can be printed using any printer. To demonstrate this capability, 3 different food colors were added to foams and added to a syringe one after another, and used to print a continuous filament printing (FIG. 13(e-iii)). The printed structure showed that the bioinks with different colors did not mix with each other under printing pressure or during the extruding process. It is noteworthy that only slight mixing of the colors happened in the transition part of the bioinks from one color to another one. This ability to avoid the mixing of reagents creates a unique opportunity to print graded scaffolds. Creating such graded scaffolds is challenging by regular hydrogel bioinks. Such graded scaffolds can be used in many applications where the interface between multiple tissues can be restored such as bone to tendon or tendon to muscle interfaces.

FIG. 13(d) shows 3D printing of foam bioink using stationary and handheld bioprinters. Rheological parameters including FIG. 13(a) storage and loss moduli, as well as FIG. 13(b) bioink viscosity were compared between the foam and gel bioinks. FIG. 13(c) shows various 3D structures printed with foam bioink using a stationary 3D bioprinter: (i, ii) zigzag and spiral printed patterns and their corresponding designs: (iii) a multi-layered (16 layers) hollow cylinder printed construct as a free-standing 3D structure. FIG. 13(d) shows 3D printing of foam using a handheld printer. (i) The custom-built handheld printer: (ii) a 3D heart shape structure printed with two different foam bioinks, followed by their in situ crosslinking using an embedded UV crosslinking light: (iii) filed syringe with 3 colors of foam bioink which did not blend to each other and continued printed filaments structure from the same syringe to show that ability of gradient printing with foam bioinks.

### Adhesion Assessment and Ex Vivo Printing

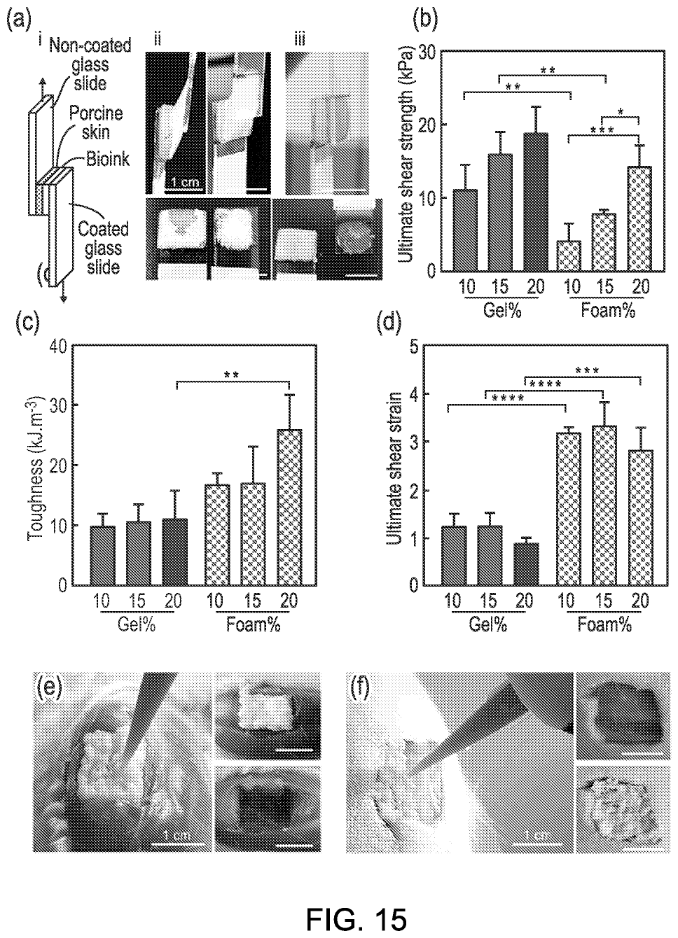

In vivo printing can offer a rapid treatment with a high level of controllability and flexibility over the printing within irregular-shaped defects. Using adhesive materials, the in situ printing and crosslinking further facilitate implantation, minimizing the requirement of fixation modalities. The application of adhesive materials for in situ printing enhances implant-tissue integration since it prevents implant slippage during body movement. Because most hydrogels are not suturable, the adhesion of the scaffold to the surrounding tissue is highly important when hydrogels are applied in regenerative medicine. Ge1MA hydrogel is known to adhere to live tissues if crosslinked in situ [37,42]. To assess the adhesion of the foam ink upon in situ crosslinking, the adhesion strength of the bioinks to natural tissues (porcine skin) was measured using a lap shear experiment (FIGS. 5a and 5b). The results demonstrated that all of the foam samples broke from the bulk structure rather than the tissue-scaffold interfaces, indicating a cohesion failure mechanism (FIG. 15(a-ii)). Therefore, the ultimate adhesion strength of the foam bioinks increased from 4+3 kPa to 6+1 kPa, and 14 +3 kPa, by increasing the concentration of Ge1MA from 10% to 15% and 20% (w/v), respectively (FIG. 15(b)). On the other hand, the gel samples failed at the hydrogel-tissue interface, showing an adhesion failure mechanism (FIG. 15(a-iii)). The results showed that the ultimate adhesion strength of Ge1MA was higher than foam. To better evaluate the mechanical behavior of the foam and gel bioinks, shear strain and toughness were also calculated in lap shear experiments (FIG. 15(c) and FIG. 15(d)). The outcomes revealed that although the ultimate adhesion shear strength of the foam is less than Ge1MA hydrogels, the total dissipated energy during the foam sample failure was higher than gel samples (FIG. 15(c)). A higher shear strain of the foam samples compared to gel further confirms the previous results and demonstrates higher flexibility of foam compared to gel (FIG. 15(d)). This indicates that the foam has a higher ductility compared to gel, which could make the foam a good candidate for engineering soft tissues such as skin and muscle.

The feasibility of the in vivo printing process and adhesion of the printed structure to the surrounding tissue were further evaluated ex vivo in a rat model. Volumetric muscle loss (VML) and skin wound injury models were utilized on euthanized rats, and a handheld printer was used for in situ printing of the foam bioink directly within the defect site (FIG. 15(e, f). The printing procedure confirmed that foam in vivo printing using handheld printers can form scaffolds in a layer-by-layer fashion to create a 3D structure, which adheres to the injury site and matches the irregular shape of the injured tissue.

FIG. 15 shows an evaluation of the foam bioink adhesion to the body tissues. FIG. 15(a) shows lap shear experiment set up, (i) schematic representation of the lap shear test, (ii) foam bioink stretched under shear and ruptured from the bulk foam (cohesion failure mechanism), compared to (iii) the gel samples failed at the tissue-scaffold interface. FIG. 15(b) shows the ultimate adhesion strength of the bioinks to porcine skin. FIG. 15(c) shows toughness and (d) ultimate shear strain of different foam and gel scaffolds, calculated from the lap shear test. FIG. 15(e) shows ex vivo printing of the foam bioinks into induced VML and (f) wound injury models to show the feasibility of foam in vivo printing and its adhesion to the surrounding tissue.

### In Vitro Biological Characterization

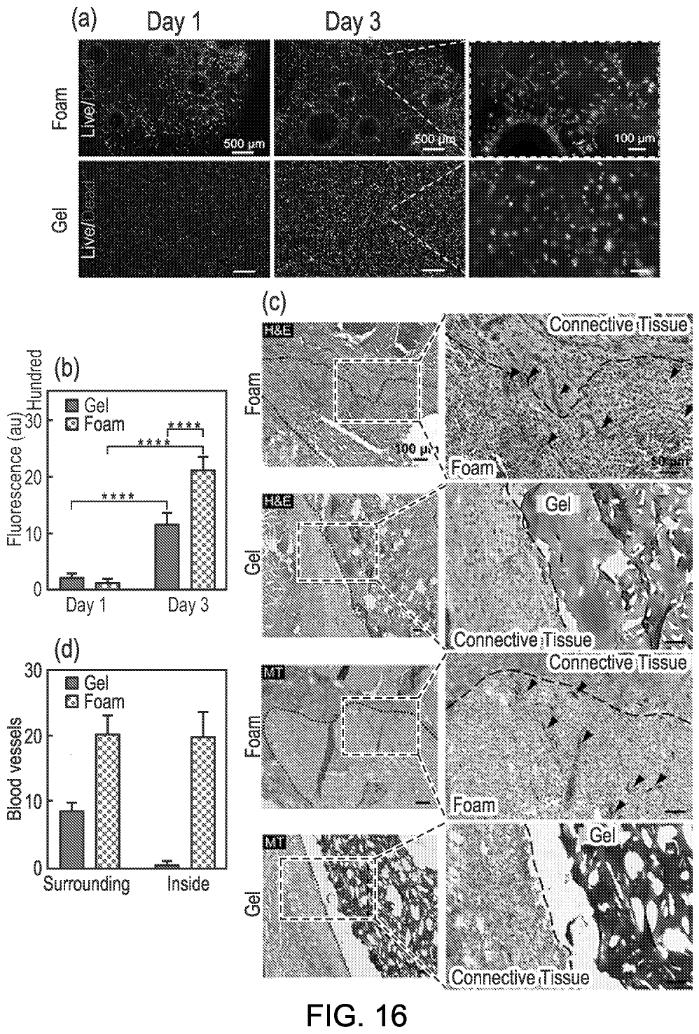

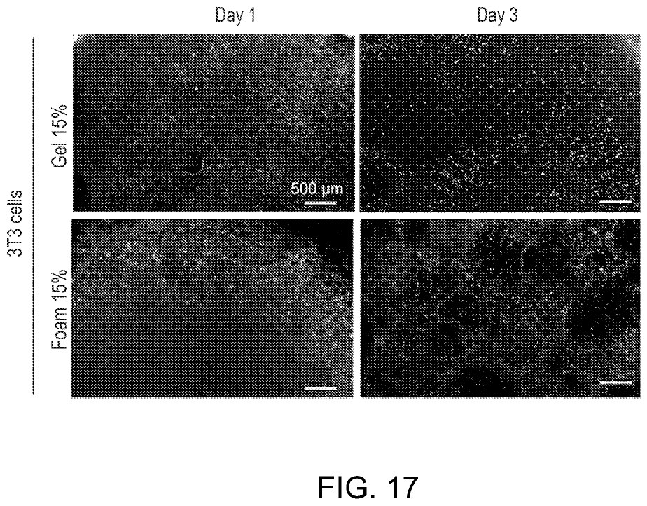

The physical and mechanical characterizations suggested that the constructs printed using 10% (w/v) foam bioink were not stable enough and did not offer sufficient mechanical and adhesion strengths for most tissue engineering applications. On the other hand, while 20% (w/v) foams were stable and adhered well to the tissues, a lower polymeric concentration in bioinks is usually preferred for better cell permissibility. Therefore, 15% (w/v) foam bioink was selected as the best concentration and its biocompatibility was assessed both in vitro and in vivo. To evaluate the biocompatibility of the scaffolds printed using foam bioink, the viability and the metabolic activity of cells were investigated using Live/Dead R and PrestoBlueR assays, respectively. Both primary (human mesenchymal stem cells (hMSCs)) and NIH 3T3 cells were considered for in vitro biocompatibility assays (FIG. 16(a) and FIG. 17). Cells were encapsulated in 15% (w/v) foam and normal hydrogel bioinks, printed on TMSPMA-treated slides, crosslinked, and incubated for up to 3 days. The micrographs of the samples stained with a live/dead assay kit demonstrated that most cells were alive in both foam and normal hydrogel scaffolds after one and three days of culture (FIG. 16(a) and FIG. 17). The staining further demonstrated that the cells were spreading in the foam scaffolds, while they remained round in gel samples after three days of culture. This indicates that the foam provides a better environment for cells to spread in compared to gel. The proliferation and metabolic activity of cells were also assessed by PrestoBlue assay (FIG. 16(b)). In accordance with live/dead staining evaluation, the metabolic activity results demonstrated a superior proliferation in foam samples compared to gel scaffolds. We postulate that cells encapsulated in foams receive more nutrients and oxygen that help them to proliferate. Moreover, a higher surface-area-to-volume ratio in foam structure supports cell expansion, without the need for scaffold remodeling, which can enhance cellular activity.

### In Vivo Study

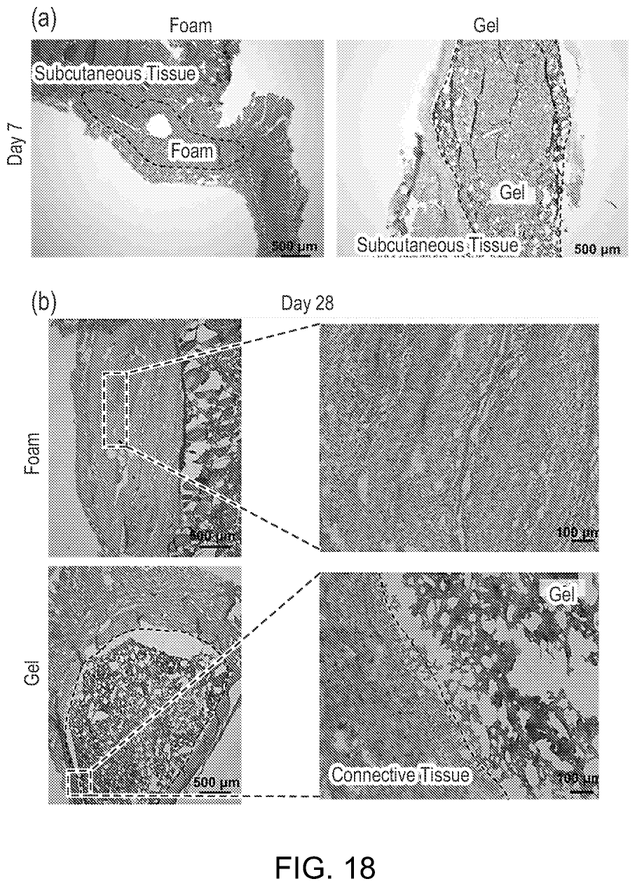

To investigate the biocompatibility of foam constructs and their ability to support regeneration in vivo, both foam and gel scaffolds were implanted subcutaneously in rats and monitored for 4 weeks (FIG. 16(c) and FIG. 17c and FIG. 18(a)). The explanted foam constructs underwent a significant reduction in the bulk size compared to gel samples. The micrographs from hematoxylin and eosin (H&E) as well as Masson's trichrome (MT) staining displayed mild inflammation in both implanted samples and their surrounding tissue including the presence of multinucleated giant cells, macrophages, fibroblasts, and collagen. The histology results showed that all the foam constructs were fully occupied by cells that infiltrated into the scaffolds after one week of implantation. Furthermore, a high number of microvessels within the foam scaffolds and their surrounding tissue were noticeable (FIG. 6(d)). Interestingly, many of the detected vessels inside the harvested scaffolds at day 7 were mature and the red blood cells were apparent inside them. However, rare infiltrative cells can be detected inside the gel samples. Additionally, the gel scaffolds were intact, and could not integrate with the host tissue. Four weeks post implantation, foam samples were fully degraded, and new tissue was regenerated. While the samples could not be found at day 28, H&E images from the tissue of the implanted area are provided in FIG. 18(b). On the other hand, the gel samples were still present in subcutaneous tissue as a bulk scaffold. A slight degradation was observed in the gel scaffolds and a thin layer of cell infiltration into the samples was detected. These results indicate that the multiscale interconnected pores in the foams can significantly increase cell infiltration, vascularization, and regeneration.

FIG. 16 shows in vitro and in vivo biocompatibility assessments of the foam and normal gel scaffolds. (a) Live/dead staining demonstrating the viability of the cells (live: green, dead: red) encapsulated cells in the foam and gel scaffolds at day 1 and 3 post printing. (b) The results of the PrestoBlue assay indicated the cells proliferation profile cultured for 3 days in foam and gel scaffolds. (c) Hematoxylin and eosin (H&E) and Masson's trichrome (MT) staining of the scaffolds-tissue interface one-week post implantation. Black arrowheads pointing to neovessels formed in the foam scaffold, demonstrating the high level of foam-tissue integration. (d) Quantitative evaluation of tissue vascularization in surrounding tissue and inside the implanted scaffold one-week post implantation.

### DISCUSSION

3D bioprinting technologies have rapidly grown over the last decade which in the conjugation of the advancements in bioink development have enabled the biomanufacturing of biomimetic tissue-like constructs [43]. Among different bioprinting approaches, extrusion 3D bioprinting of hydrogel scaffolds has received special attention due to its compatibility with a wide range of bioink materials and cell densities [12]. An ideal bioink for 3D bioprinting can offer good printability for the fabrication of 3D constructs with high fidelity, while it needs to provide a cell favorable environment supporting their migration, proliferation, and maturation. Among various hydrogel inks, alginate has been widely used due to its favorable rheological properties and fast crosslinking in the presence of divalent cations [44]. However, alginate does not properly support cellular activity because of lacking the cell adhesive moieties and dense polymeric network [45,46]. Collagen, as the main component of ECM, has a suitable biological efficacy, though its delayed crosslinking has limited its application in bioprinting to very simple planar constructs [47]. Therefore, a hydrogel with optimal printability and biocompatibility is advantageous for extrusion-based 3D bioprinting.

In recent years, Ge1MA has attracted increasing attention for tissue engineering applications as a result of its biocompatibility and ease of photocrosslinking [36,48]. However, its low viscosity and relatively long crosslinking time limited its application in extrusion-based 3D bioprinting. Several strategies have been used to improve the printability of Ge1MA such as increasing the precursor concentration, partial pre-crosslinking to increase its viscosity, and integration of other hydrogels such as alginate and gelatin to improve the rheological properties and facilitate the rapid crosslinking of the hybrid hydrogel [49,50]. However, increasing the precursor concentration results in reduced cellular activity inside the Ge1MA scaffolds, partial pre-crosslinking makes the bioprinting process unreliable, and hybridization complicates the bioink preparation, printing, and crosslinking, all reducing the printability window [51,52]. Another strategy is the incorporation of nano- or micro-particles to improve the rheological properties of Ge1MA and make it shear thinning, though it has been reported that this process can significantly affect the cell ingrowth inside the scaffold [46]. Additionally, limited diffusion of nutrients, oxygen, and waste through the small microporous hydrogel network further diminish the cellular activity inside the conventional Ge1MA-based scaffolds, specifically when constructs with clinically relevant dimensions are important [53]. It has been reported that the diffusion within hydrogel constructs is very slow and inefficient in millimeter-scale and larger constructs [54]. The small pore size in the Ge1MA structure further limits the cell infiltration, expansion, and therefore final tissue integration

To resolve the above-mentioned challenges of Ge1MA bioprinting, here we introduce multiscale porosity inside the hydrogel precursor to enhance both the printability and cell permissibility of the bioprinted Ge1MA scaffolds. Inspired by whipped cream production in pastries, we developed porous bioinks through a simple foaming process with high-speed stirring. In this approach, the pore size can be controlled by varying the stirring duration, stirring speed, and concentration of the hydrogel. The Ge1MA foaming process led to the introduction of spherical bubbles of 50-400 μm size, surrounded by a naturally microporous Ge1MA network. Our results demonstrate that the mechanism of pore formation in the stirring process is shear-induced bubble splitting. In this mechanism, large air bubbles introduced initially by inserting the tip of the homogenizer inside the solution are split into smaller bubbles due to the shear stress between the solution layers where the bubble is located [55]. The shear stress dominates the surface tension of the bubble and forms smaller independent bubbles consecutively until the bubbles are smaller than a critical size to be split with shear stress applied to the bubble surface. Increased Ge1MA concentration and stirring speed directly increase the shear stress, decreasing the bubbles' lower limit size, while increasing the stirring time can offer a more homogeneous pore size dispersion. Overall, our results demonstrate that a 40 sec stirring with 22000 rpm can form an almost homogeneous porous structure in 15% Ge1MA, with a 75 μm average pore diameter. Since foam instability caused by bubble merging has been a concern in air colloidal systems, PVA, a biocompatible water-soluble polymer, is implemented here to enhance the stability of bubbles in the foam and decrease the bubble burst rate during the foam generation process [56,57]. Micrographs from printed filaments of Rhodamine B loaded Ge1MA foam containing PVA after their incubating in an aqueous environment, demonstrated the formed bubbles during the foaming process, extrusion, and post-crosslinking were stabilized.

The rheological data from foam and gel bioinks showed that the foam experience much smaller moduli (storage and loss) changes during sol-gel transitions compared to gel making its bioprinting parameters more stable and reproducible. Furthermore, the results demonstrated that the viscosity of the foam bioink was much higher than the gel, due to internal friction induced by the presence of packed bubbles, making it much easier to print. Foam bioink was robust and not only could be printed using stationary and fully automated bioprinters, but it also offered rheological properties that one could print and write structures using a handheld device. The ability to print the ink using a handheld printer means that it can be used for direct in vivo printing of scaffolds in the defect site. This approach has drawn significant attention recently because this strategy offers a rapid, simple, and controllable treatment procedure while it can overcome the challenges associated with the implantation and fixation of the scaffolds within the defect through the application of adhesive materials. Our lap shear experiments demonstrated that the foam bioink has a proper adhesion strength (˜6-14 kPa) to the skin tissue and has more flexibility and ductility compared to the gel. This indicates that the foam is a suitable bioink for in vivo printing in soft tissue injuries. The foam bioink offered high shape fidelity, structural stability, proper mechanical properties for soft tissue engineering [40], and strong adhesion to the surrounding tissues in vitro and ex vivo.

In addition to printability, a bioink needs to support cellular activity and good tissue integration. To evaluate this, we investigate the biocompatibility of the bioinks in vitro and in vivo. Our in vitro live/dead staining, as well as metabolic activity assay results, demonstrated that the foam bioink, in contrast to the gel with a similar polymeric concentration, completely supports cellular viability, spreading, and proliferation. These improvements are probably because of the meso- to micro-porous structure within the polymeric networks, which provide enough space for cell expansion and proliferation [58]. The in vivo evaluation of foam bioinks after subcutaneous implantation in rats further indicated a complete implant tissue integration recognized by a high level of cell infiltration into the scaffolds after one week of implantation, as well as matured vascularization within the foam scaffold and its surrounding tissue. The rapid foam scaffold remodeling in contrast to the gel can be attributed to the presence of interconnected mesoscale and microscale pores in the scaffold structure. The multiscale interconnected porous structure offers a larger space for the cells to anchor and expand, making the foam a suitable environment for improved cell adhesion, differentiation, and tissue regeneration. It has been reported that the interconnected structure facilitates cell infiltration and transport of nutrients and waste, and therefore accelerates scaffold-tissue integration and its vascularization and innervation [59].

### Materials:

Materials were purchased from Sigma-Aldrich unless mentioned otherwise. Ge1MA was synthesized as per a previously published protocol. In brief, 10% (w/v) gelatin derived from porcine skin (Sigma-Aldrich) was dissolved in DPBS (HyClone) at 50° C. Next, 8% (v/v) methacrylic anhydride (Sigma-Aldrich) was added dropwise to the gelatin solution under stirring (300 rpm). The reaction was stopped after 3 hours by diluting the solution with preheated DPBS. The solution was dialyzed in dialysis tubing (Spectrum Laboratories, MWCO 12-14 kDa) against deionized water at 50° C. for 7 days to remove any unreacted methacrylic anhydride. The solution was filtered followed by 6 days of lyophilization to form a white powder.

### Bioink Preparation

For preparing foam bioinks, at first, 1% (w/v) PVA was dissolved in Dulbecco's phosphate buffered saline (DPBS) (HyClone) at 100° ° C., after cooling down to 37° ° C., 0.3% (w/v) lithium phenyl-2,4,6-trimethylbenzoylphosphinate (LAP) (Sigma) was dissolved in the solution and covered with foil to be protected from light. To prepare different concentrations of Ge1MA solution, 10%, 15%, and 20% (w/v) of Ge1MA were dissolved in the solution. Ge1MA solutions were poured into a 5 mL syringe closed beforehand with a locking tip and were homogenized using a homogenizer (SCILOGEX D160) at different speeds for a specific time. The foam bioink inside the syringes was used for bioprinting. The same Ge1MA solutions were used to form hydrogel scaffolds as a control for all experiments. Printed structures were then crosslinked with visible lights using a light-emitting diode (LED) light (395 nm −400 nm, 20 W) for 1 min.

Characterization of Porosity and Pore Size Distribution within the Foam Bioinks:

### Scanning Electron Microscopy (SEM)

SEM was performed to evaluate the structure and pore size distribution inside the printed foam structures. The foam bioink was prepared from 10%, 15%, and 20% (w/v) Ge1MA containing 1% (w/v) PVA and photo-initiator, which were foamed using 20, 40, and 60 sec of homogenization at 8,000:15,000; and 22,000 rpm. The prepared bioinks were then printed on glass slides followed by photocrosslinking for 1 min. The printed scaffolds were immediately immersed in liquid nitrogen to snap freeze the hydrogel and subsequently lyophilized (FreeZone 2.5 Liter −50° C. Benchtop, Labconco) for 24 h. The samples were then broken and the exposed internal microstructure was coated with a thin gold layer for 60 sec at 20 mA using a sputter coater device (Vacuum Desk V, Denton). Finally, the samples were imaged using a benchtop SEM device (TM-1000, HITACHI).

To perform image analysis and quantify the pore size distribution, the area of pores was measured in ImageJ software and divided by x to obtain the average pore diameter. The data from three images per group were pooled and a normal distribution was fitted on the data using the built-in function in Microsoft Excel software.

### Density and Porosity Volume Calculation

In order to investigate the density and porosity of foam bioinks, EPPENDORF® tubes were filled with precursor gel and foam bioink until a defined volume. Each tube was weighed before and after filling with bioinks, and the density (p) of each concentration was calculated through this equation:

$$ρ = \frac{{W}_{s a m p l e}}{{V}_{t o t a l}}$$

where Wₛₐₘₚₗₑ is the weight of samples determined from the weight difference of each tube before and after filling with samples and Vₜₒₜₐₗ is the total volume that filled in the EPPENDORF® tubes.

Then the percentage of porosity φ_{foam} (%)) in the foam was calculated from the density results through this equations:

$${V}_{pores in foam} = {V}_{total} - \frac{{W}_{foam}}{{ρ}_{GelMA}}$$

$${φ}_{foam} ( \% ) = \frac{{V}_{pores in foam}}{{V}_{total}} \times 100$$

where W_{foam} is the weight of foam samples in the EPPENDORF® tubes that was measured initially, ρ_{Ge1MA} is the density of Ge1MA that is calculated from equation (1), and V_{pores in the foam} is the volume of the pores existing in the foam samples.

To evaluate the foam structure and the pores after printing in physiological conditions and its stability in an aqueous environment, Rhodamine B (˜8 mg/ml) was dissolved in the prepared Ge1MA solution and homogenized with a homogenizer, as outlined before, to prepare the foam ink, then used to print. The printed structures were kept in DPBS solution, then at each time point, the samples were taken out from DPBS and were imaged with a Zeiss fluorescent microscope.

In Vitro Swelling

Samples were prepared with the same geometry (10 mm L×5 mm W×2 mm H) and crosslinked for 1 min with visible light. The original weight of samples measured and then were immersed in DPBS and incubated at 37° C. for a total of 48 h. At each time point, swollen samples were removed from the DPBS solutions, blotted with tissue paper to remove excess solution, and weighed. The swelling ratio (SR %) was determined using the following equation:

$$S R ( \% ) = \frac{{W}_{1} - {W}_{0}}{{W}_{0}} \times 100$$

where W₀ is the initial weight of the samples and W₁ is the weight of each swollen sample at each time point.

### In Vitro Mass Change Measurement

The mass change of samples was investigated in two conditions: 1) DPBS and 2) DPBS containing 20 μg/mL of collagenase I to mimic an enzymatic degradation of gelatin-based materials in vivo. The original weight of samples measured and then were immersed in the solutions and incubated at 37° C.(for 5 days in collagenase solution and 42 days in DPBS). At each time point, samples were removed from the solutions, blotted with tissue paper to remove excess solution, and weighed. The weight change ratio (WR %) was calculated according to the following equation:

$$W R ( \% ) = \frac{{W}_{1}}{{W}_{0}} \times 100$$

Where W₀ is the initial weight of the samples and W₁ is the measured weight of samples at each time point.

### Compression and Cyclic Compression Tests of the Scaffolds

To investigate the mechanical properties of foam bioinks and compare them with gel, compression and cyclic compression tests were performed on samples. Specimens were prepared by injecting the precursor gels and foam bioinks in the cylindrical shape molds (6 mm diameter and 5 mm height) made of polydimethylsiloxane (PDMS) and crosslinked with visible light for 1 min. The dimension of samples was also measured by caliper after molding for accurate calculation. An INSTRON 5542 mechanical tester was used to perform the experiments and cylindrical samples were loaded between the compression plates. Compression tests were conducted at a strain rate of 1 mm·min⁻¹. The compressive moduli of the specimens were calculated from the slopes of loading stress vs strain curves at the first 10% of the strain. Cyclic compression tests were conducted with a rate of 1 mm·min⁻¹ at a 50% strain level by performing 20 cycles of loading and unloading (only cyclic cycle 10 is plotted in FIG. 4e). Energy loss (EL %) was calculated based on the area between the loading and unloading curves at cycle 10 (n≥5):

$$EL ( \% ) = \frac{{A}_{l o a a i n g} - {A}_{unloading}}{{A}_{loading}} \times 100$$

Where A_{loading} is the total area under the loading curve and A unloading is the total area under the unloading curve at cycle 10.

### Rheological Analysis

Rheological characterization of the bioinks was performed using a Discovery HR-3 hybrid rheometer (TA Instruments, USA). A 40 mm flat plate was mounted on the device head, the bioink was loaded and oscillation was performed at 1% strain and 1 Hz frequency using an 800 μm gap. A “temperature ramp” protocol was used to decrease the temperature of the bioink from 37° ° C. to 4° C. over time and collect the corresponding storage and shear moduli, G′ and G″. The complex viscosity was then calculated using η=(G′²+G″²)^{1/2}ω, in which w is the angular frequency.

### Bioink Printing

Foam bioinks were prepared as outlined before into a 5 ml syringe and the luer lock tip was replaced with a 25 G tapered plastic tip for printing. The syringe was loaded into an Allevi 3R 3D bioprinter for performing the computerized printing. The printability of the foam bioink was investigated under various ranges of temperature, pressure, and speed to find the optimum printing parameters. The bioinks were maintained at 22° C. in the 3D printer and during the printing. The optimum extrusion pressure and printing speed were defined at 9 PSI and 5 mm·sec⁻¹, respectively. Different structures were printed with a layer height of 260 μm with custom G-codes. Then the printed structures were crosslinked by exposing them to visible light for 1 min.

For evaluating the printing of foam bioinks through the partially automated custom design handheld printer, foam bioinks were prepared as described before in a 5 mL syringe and then injected into a 3 mL syringe that could fit the handheld printer. The printing was performed with a 22-gauged tapered plastic tip for making different structures at low-medium speed (the extrusion rate is from 0 to 10 μL/s). The printed structures were exposed to the embedded ultraviolet (UV) cross linking system. The liquid food color was added to the foam bioink to make the structures more visualized.

To look into the ability of gradient printing with the foam bioinks, foam bioinks were prepared in 3 different syringes, and each one was mixed with food color. Foam bioinks with different colors were injected into one syringe sequentially and used to print with the handheld printer in a continuous line printing in a single layer structure.

### Shear Adhesion Testing

The lap shear strength of the bioinks to natural tissue was assessed according to a modification of the standard test method ASTM F2255-05 [60]. The 3-(trimethoxysilyl) propyl methacrylate (TMSPMA: Sigma-Aldrich) and non-coated glass slides were cut into 13 mm×35 mm rectangles. Porcine skin tissues were cut into rectangular samples (13 mm×10 mm×1 mm) and glued to the non-coated glass slide. The bioinks were printed in rectangular samples (13 mm×10 mm×2 mm) on the porcine skin, the TMSPMA glass slide was placed in touch with the printed structures, and the bioink was crosslinked for 1 min with visible light. The tests were conducted using an Instron 5542 mechanical tester at a shear rate of 1 mm·min⁻¹ until the samples detached or ruptured (n≥5).

### Ex Vivo Printing on Tissue

To evaluate the practicality of the printing process and adhesion of the printed structure to the surrounding tissue, a rat model of VML and skin wound injury was utilized on euthanized rats as previously described and the handheld printer was used for in situ printing of the foam bioink directly within the defect site. The handheld UV crosslinker was activated during the printing to crosslink the printed layers.

### In Vitro Biological Characterization

Human mesenchymal stem cells (hMSCs) were purchased from ATCC and cultured at 37° C., and 5% CO2 in mesenchymal stem cell basal medium (ATCC, PCS-500-030) supplemented with a mesenchymal stem cell growth kit (ATCC, PSC-500-041) and 1% (v/v) penicillin/streptomycin (Gibco). The media changed every two days and cells were passaged at 80% confluency. NIH 3T3 cells were also cultured with Dulbecco's Modified Eagle Medium (DMEM)(Gibco) media supplemented with 10% (v/v) fetal bovine serum (FBS) (HyClone), and 1% (v/v) penicillin/streptomycin. To evaluate the cell behavior and their expansion in the bioinks, cells were harvested through trypsin-EDTA (0.1%) incubation. Then a 3×10⁶ cell.mL⁻¹ suspension of cells centrifuged and the cell pellet resuspend in 30 μL of media and added to a 1 mL of bioinks. Cells were encapsulated and mixed through pipetting in the solution of 15% Ge1MA hydrogel and mixed with a spatula in 15% Ge1MA foam as bioinks. The small amount of bioinks (˜ 80 μL) extruded from the handheld printer on the TMSPMA coated glass slides (8 mm×8 mm), crosslinked under visible light for 1 min, washed with DPBS, transferred to 24-well plates, and incubated with media at 37° C., and 5% CO2.

The viability and expansion of encapsulated cells were investigated through LIVE/DEAD™ Viability/Cytotoxicity Kit (Invitrogen) on day 1 and day 3. According to the manufacturer's instructions 2 μL. mL⁻¹ Ethidium homodimer (EthD-1) and 0.5 μL.mL⁻¹ calcein-AM diluted in DPBS. At each time point cell encapsulated samples were incubated with the solution for 20 min at 37° C., and 5% CO2. After the incubation time, samples were washed with DPBS, and cells were imaged by a fluorescence optical microscope (Primovert, Zeiss) which live and dead cells appear in green and red fluorescence colors, respectively.

### Animal Study

The in vivo study was approved by the ICAUC (protocol #2018-076-01C) at the University of California Los Angeles (UCLA). Wistar rats (Male: 200-250 g) were obtained from Charles River Laboratories (Boston, MA, USA). For anesthesia, isoflurane (2-2.5%) was used. After anesthesia, one-cm incisions were made on the rat dorsal skin, and small pockets were created using a blunt surgical scissor. Gel and foam scaffolds were formed using a cylindrical mold, crosslinked with visible light for 1 min, and sterilized under high-intensity UV light for 10 min. After implantation of the samples, incisions were sutured with 3-0 polypropylene sutures (EthiconR). In the first 48 hours, the physical state of the rats was observed twice per day. Carprofen was postoperatively administered prior to making the incision and also 24 hours after the surgery (5 mg/kg).

### Histological Analyses

At days 7 and 28 post implantation, the animals were euthanized and the samples were explanted with the surrounding connective tissues for histological studies and fixed in 4% paraformaldehyde for 24 h. Samples were then incubated in 15% sucrose for 4h and 30% sucrose for 24 h at 4° C., sequentially. Then, samples were embedded in an Optimal Cutting Temperature compound (OCT), the sample blocks were immersed in 2-methylbutane in a beaker and flash-frozen in liquid nitrogen. Frozen samples were sectioned (8 μm) utilizing a LEICA BIOSYSTEMS CM1900 Cryostat and mounted on positively charged slides. The cryosectioned samples on slides stained with hematoxylin and eosin (H&E) (Sigma) and Masson's trichrome (Sigma) according to instructions from the manufacturer. The stained sample slides were fixed with DPX mounting medium (Sigma) and imaged using the brightfield of a Zeiss microscope.

### Statistical Analysis

Displayed data are reported as mean±standard deviation. GraphPad Prism 8.0 software (San Diego, CA) was used to perform statistical analyses. Column analyses were conducted using a two-tailed Student's t-test. Grouped analyses were completed using analyses of variance (ANOVA) testing. Significant ANOVA results underwent Tukey's multiple comparisons post-hoc testing. Values of p<0.05 were considered statistically significant. p<0.05 denoted by *, p<0.01 denoted by ** , p<0.001 denoted by *** , and a p <0.0001 denoted by ***.

### Example 3

Volumetric muscle loss (VML), which is a composite defect of skeletal muscle, does not heals primarily with scar, minimal muscle regeneration, and leads to permanent disability. Current surgical and physical therapies are inadequate and therefore regenerative therapy is needed to effectively treat the numerous patients suffering from VML. Despite the success of acellular scaffold-based therapies in improving the maintenance of the remnant muscle, they have failed in inducing robust muscle regeneration. In this study, following VML, a significant functional loss of muscle was found to be accompanied by a down-regulation of insulin-like growth factor 1 (IGF-1), an important factor responsible for muscle maintenance and regeneration. In light of this, a colloidal scaffold with hierarchical porosity that sustains efficacious levels of recombinant IGF-1 was engineered. The foam-like scaffold was in situ printed using a handheld 3D printer, which led to its adhesion to the remnant muscle without the need for suturing. The suitable concentration of IGF-1 to have a regenerative effect was identified in vitro. The scaffold offered an advantageous compressive modulus and an advantageous adhesion strength. Post implantation, the foam-like scaffolds carrying IGF-1 significantly improved functional recovery. Histological analysis confirmed the generation of new muscle tissue in the foam-like scaffolds, especially in the group carrying IGF-1. In addition, the scaffolds significantly reduced fibrosis and increased the expression of neuromuscular junctions in the newly regenerated tissue. Exercise therapy in the animals with VML injury did not lead to significant functional recovery: however, in combination with the foam-like scaffolds, physical therapy augmented the treatment outcome in a synergistic fashion.

### 1 Introduction

Skeletal muscle enables movement, protects internal organs from impact, and participates in vital actions such as chewing and maintaining temperature homeostasis ¹⁻³. Skeletal muscle possesses a high regenerative capacity to heal minor injuries, which is necessary to maintain its mass and strength ^{4,5}. The natural regeneration of the skeletal muscle happens through a cascade of physiological events orchestrated by resident satellite cells ⁶⁻⁸. Following injury, activated muscle stem cells proliferate, differentiate into myocytes, and then either fuse together to form new multinucleated muscle fibers or fuse to existing myotubes and promote hypertrophy 9. However, volumetric muscle loss (VML) injuries, which are composite skeletal muscle injuries, overwhelm the regenerative potential of muscle. These injuries, especially when they comprise 20% or more of the muscle belly, heal with poor regeneration and extensive fibrosis, resulting in chronic functional deficits ^{10,11}. Following VML, downregulation of myogenic factors and upregulation of profibrotic factors all limit strength and functional recovery, especially in the absence of scaffold placement ^{12,13}.

Therapeutic options for VML injury remain limited. Currently, free tissue transfer of functional muscle is the standard treatment for substantial VML injuries ¹⁴⁻¹⁶. However, this approach leads to limited functional recovery and donor site morbidity ¹⁶. Targeted physical therapy may also improve recovery following VML but its benefit is limited ¹⁷. Tissue engineering offers an alternative strategy to VML treatment. An engineered muscle graft for promoting skeletal muscle regeneration can be constructed by the integration of myogenic factors and cells into scaffolding materials that mimic the native extracellular matrix ^{1,18,19}. A promising method to fabricate such scaffolds as a replacement for the lost tissue is 3D (bio)printing ^{1,20,21}. 3D (bio)printing is used as an additive manufacturing strategy through which bioinks are deposited in a controlled manner for the biofabrication of tissue-like constructs ²². 3D (bio)printing can fabricate complex muscle grafts with clinically relevant sizes ²³. However, this strategy suffers from a number of challenges, namely the need for scanning modalities to reproduce the defect morphology, computer-aided design and manufacturing (CAD/CAM) tools and expertise, and a properly isolated environment to prevent potential infection ^{24,25}. These standard technologies and requirements result in delayed implantation, limited tissue integration due to the low tissue adhesion, and challenges for conformation into curved irregular defects ^{24,25}. One strategy that can overcome some of these limitations is the direct printing of the scaffolds within the patient's body, named in vivo or in situ printing ²⁵. In situ printing can be performed using a computer-controlled system depositing a bioink directly into a defect ²⁶. However, this approach still requires scanning and CAD/CAM implementation, along with sophisticated robotic systems.

To address the current issues regarding the use of scaffolds as a therapeutic option for the treatment of VML, we have developed handheld (bio)printers for in situ printing of scaffolds for skin ²⁴, bone ²⁷, and muscle ^{28,29} regeneration. The in situ printing using this strategy is rapid, and actively controlled by the surgeon during the operation. This eliminates the requirement of CAD/CAM systems and can be conformed to irregular defects with curved surfaces. Furthermore, the in situ crosslinking of bioink upon its deposition generally enhances its tissue adhesion and therefore graft integration. However, functional recovery of injured muscle treated with such a scaffold was previously shown to be limited by poor tissue ingrowth and limited induction of muscle regeneration within the scaffold itself. These results support the delivery strategy but highlight the need for bioinks with specific properties to support both biofabrication and tissue regeneration. Ideally, 3D scaffolds can recapitulate the biological and physical properties of the extracellular matrix to assist with muscle regeneration, support cellular infiltration, proliferation, and differentiation, as well as promote the distribution of nutrients and oxygen ³⁰⁻³². While significant effort has been put into the engineering of various cell-permissive scaffolds, the dense polymeric network of printable bioinks usually limits normal cellular behavior and affects their migration, proliferation, and maturation. This in turn leads to poor myogenesis, vascularization, and innervation ³³⁻³⁵. Limited diffusion of nutrients into these scaffolds further impedes cellular activity, especially within larger constructs that are required for VML ^{36,37}. A possible solution for this is to incorporate hollow channels within the scaffold through multimaterial bioprinting ^{38,39}. However, this strategy is complex and can be challenging to be implemented for in situ printing as such porosity negatively impacts mechanical properties, fidelity, and structural stability of the printed construct.

The engineered scaffold can be supplemented with myogenic factors to enhance the regenerative response of injured muscle. Various biochemical factors, mainly provided by immune cells and platelets, have been reported to contribute to different stages of muscle regeneration ^{8,40}. One of the most notable myogenic factors in muscle regeneration is insulin-like growth factor-1 (IGF-1), which is known to promote satellite cell proliferation and differentiation, as well as immune modulation ⁴¹. However, exogenous IGF-1 therapies have clinically failed due to toxicity and adverse effects of systemic delivery, and difficulty in maintaining the therapeutic concentration of IGF-1 at the injury site after bolus injections ^{42,43}. Therefore, a delivery system that allows localized, sustained release of IGF-1 within injured skeletal muscle is important for attaining adequate myogenic effect of the growth factor ⁴⁴.

Here, we develop a simple and clinically implementable strategy to address the above-mentioned requirements for both the biomaterial ink as well as biofabrication and implantation approach. We have engineered frothy scaffolds with multi-scale porosity to enhance cell permissibility, carrying IGF-1-loaded microparticles to enable its sustained release for enhanced myogenesis. An in situ printing strategy was utilized to deliver the scaffold directly into muscle defects of a murine VML model. This scaffold was designed to adhere to the tissue directly and offer a temporary 3D myogenic microenvironment for cellular infiltration, proliferation, and differentiation toward the restoration of muscle structure and function. Lastly, as regimented exercise may promote functional recovery as well ⁴⁵, we demonstrated that exercise therapy combined with the acellular, foam scaffold offers the most complete recovery following VML. Given the acellular nature of the scaffolds, it holds promise for rapid translation into clinical use for patients with VML injuries.

### 2 Results

It has been reported that post VML injury, the loss of the cellular, structural, and chemical components needed for healing prevents effective regeneration and leads to permanent loss of function ^{12,13}. Here, a murine model of VML of the posterior compartment of the leg was utilized as previously described. Evaluation of the gross images of injured muscles without any interventions confirmed minimal restoration of the lost muscle volume eight weeks following injury (FIG. 19A), indicating poor muscle regeneration. Furthermore, in vivo ankle torque measurements demonstrated approximately a 50% reduction in muscle force production immediately after VML injury, and the functional deficit remained at around 30% lower than the muscle that underwent a sham operation after 8 weeks (FIG. 19B). An enzyme-linked immunosorbent assay (ELISA) demonstrated significantly lower levels of (p=0.002) IGF-1 within the remnant skeletal muscle tissue at 3.7±2.3 ng/g in the VML injured group, as compared to 9.2±2.5 ng/g (mean±SD) in uninjured animals two weeks after VML injury (FIG. 19C). These results suggest that the loss of extracellular matrix (ECM) and diminished local level of myogenic factors associated with VML may be the cause of reduced regenerative capacity of the remnant tissue, and the shift from the regenerative mechanism to the repair mechanisms of healing such as fibrosis. Therefore, regenerative therapies may need to include both scaffolds that support tissue ingrowth and sustained release of myogenic factors, specifically IGF-1, to be efficacious.

To achieve this goal, we developed a strategy based on the in situ printing of highly porous Ge1MA-based scaffolds that could control the release of IGF-1 (FIG. 19D). In situ printing as the delivery method of this noble scaffold was elected due to its simplicity and a high potential for its translation into the clinical treatment of VML. Due to favorable cellular adhesion, biodegradability, and tunability of physical and chemical properties ^{46,47}, Ge1MA was selected as the primary biomaterial forming the bioink for in situ printing. However, the dense polymeric network of the Ge1MA hydrogel limited cellular infiltration and scaffold integration in our previous studies 29. To enhance the cellular permissibility of the scaffold, a simple foaming method, inspired by whipped cream production for pastries, was considered to introduce mesoscale pores into the Ge1MA structure. On the other hand, Ge1MA, which is mainly synthesized with type A gelatin, is positively charged at neutral pH 48 and consequently not adequate to bind to electropositive IGF-1 49. Therefore, negatively charged gelatin type B ⁴⁸ microparticles were selected to be incorporated into the structure to serve as a linkage element.

FIG. 19D illustrates a schematic overview of the designed concept and process for the treatment of VML in this study. Initially, gelatin microparticles are loaded with IGF-1 and encapsulated into a Ge1MA precursor. Subsequently, the solution is stirred at high speed to foam the biocomposite and form the final bioink. The bioink is then directly printed onto the VML defect using a handheld printer. In situ crosslinking stabilizes the printed filaments and leads to their adhesion to the remnant tissue ²⁹. The hierarchical pores were expected to enhance the cellular permissibility and sustain the release of IGF-1, thus promoting rapid proliferation and differentiation of the infiltrated cells to regenerate muscle and restore its lost function.

FIG. 19 shows a murine VML model and the proposed strategy for its treatment. (A) Gross images of the extracted muscles 8 weeks post-surgery. Lack of regeneration and smaller volume of the muscle in the VML injury group compared to the sham group confirmed the applicability of the model as a VML injury. (B) Force generation capability of the muscle post VML injury. A significant reduction was detected in the measured isometric torque immediately after defect induction, as well as eight weeks post-surgery, demonstrating the chronic deficit of the muscle post VML. (C) Assessment of IGF-1 level in remnant muscle post VML. A significant reduction in the concentration of IGF-1 was observed two weeks post VML injury. Considering the requirement of IGF-1 in natural muscle regeneration, a reduced level of this growth factor can be considered a major contributor in impaired regeneration post VML. (D) The proposed strategy for the treatment of VML in this work. Negatively charged gelatin nanoparticles are synthesized to link positively charged IGF-1 into a positive Ge1MA structure. The precursor is then foamed and directly printed into the muscle defect using an in situ printing method with a custom handheld printer. The printed scaffolds adhere to the remnant tissue and possess a mesoporous structure to facilitate cell infiltration. The release of IGF-1 then is expected to enhance the activity of infiltrated cells toward muscle regeneration and its functional recovery.

### 2.1 the Effect of IGF-1 on Muscle Progenitors

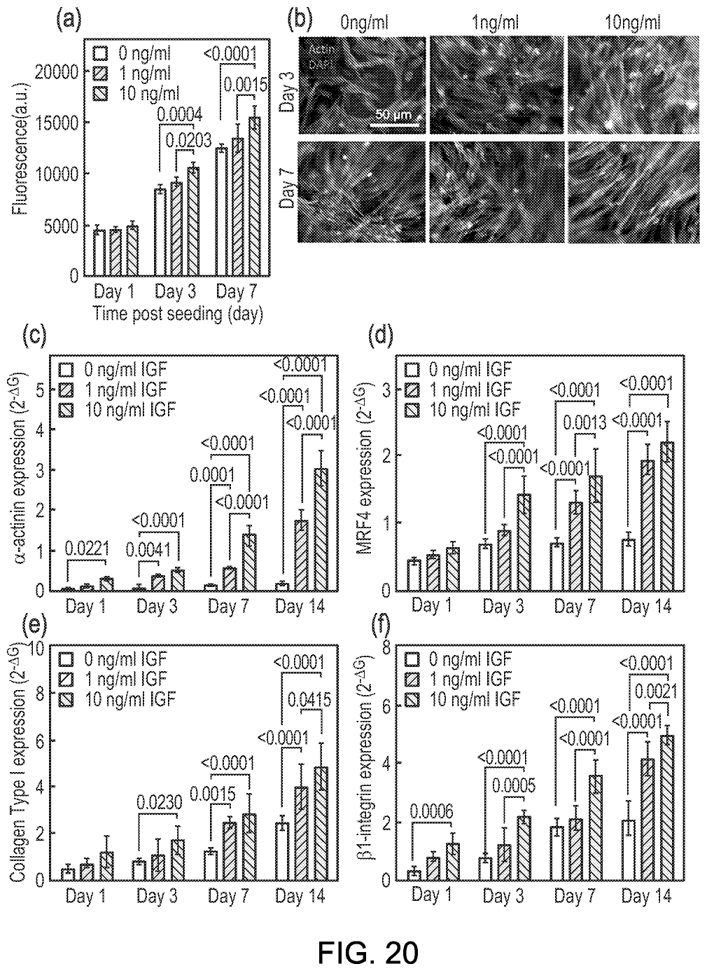

To assess a suitable range of IGF-1 concentrations to enhance cell proliferation and myogenesis, C2C12 myoblast cells were treated with different IGF-1 concentrations in vitro, after reaching 80% confluence (FIG. 20). The results demonstrated that 10 ng/ml of IGF-1, a concentration close to the physiological concentrations in muscle (FIG. 19C) had a significant effect (p=0.0004 and p<0.0001, respectively on day 3 and day 7 of the culture) on the metabolic activity of myoblasts (FIG. 20A). The F-actin/nuclei staining of the cultures showed a better alignment and organization in cultures treated with 10 ng/ml of IGF-1 (FIG. 20B). This might reflect a higher proliferation rate and therefore enhanced cellular communication between the proliferating cells in the presence of IGF-1 39.

Gene expression was assessed to further evaluate the effect of IGF-1 on myogenic differentiation. Two myogenic markers: α-actinin and MRF4, an ECM component: type I collagen, and a cell adhesion marker: β1-integrin, were selected for the assessment of the myogenic behavior of differentiating muscle cells 46.50 (FIG. 20C-F). The results demonstrated that supplementation of the cells with IGF-1 significantly promoted myogenic differentiation (FIG. 20C, D). α-actinin, a molecule that contributes to force generation was expressed around 16 folds higher when cells were exposed to IGF-1 at physiological concentrations (10 ng/ml, FIG. 20C). Similarly, the expression of MRF4, a late myogenic marker ⁴⁶ was doubled as soon as the third day of differentiation with 10 ng/ml IGF-1 (FIG. 20D).

The results further demonstrated that not only the myogenic differentiation, but also ECM deposition and cell-cell/cell-ECM adhesion were enhanced with IGF-1 supplementation at both 1 ng/ml and 10 ng/ml concentrations, but more significantly at higher concentrations comparable to the physiological level in healthy muscle (FIG. 20E, F). These results suggest that loss of IGF-1 seen following VML injury may impair skeletal muscle regeneration and differentiation within the area of muscle injury.

FIG. 20 shows in vitro effect of IGF-1 on C2C12 muscle progenitors. (A) Metabolic activity of myoblasts, exposed to different levels of IGF-1, over one week in culture condition. IGF-1 at a physiological concentration (10 ng/ml) significantly enhanced cellular proliferation. (B) F-actin/DAPI staining of the cells exposed to different IGF-1 levels on day 3 and day 7 of culture. Enhanced proliferation and consequent alignment can be observed in cells exposed to 10 ng/ml IGF on day 7. (C-F) Gene expression analysis of the cellular behavior during differentiation. The expression of two myogenic markers α-actinin (C) and MRF4 (D) demonstrated a significant improvement in cellular differentiation. The expression of Collagen I (E), an ECM protein, and β1-integrin (F), a cell adhesion molecule, was further significantly increased during differentiation.

2.2 Development and Characterization of IGF-1 Eluting Scaffolds with Multiscale Porosity

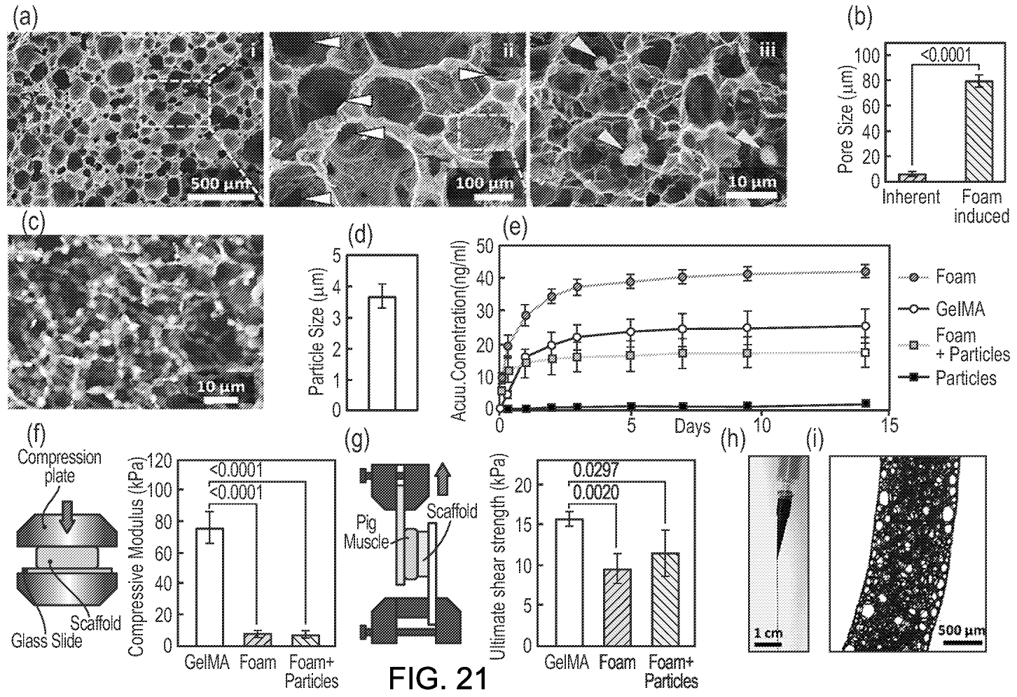

After determining the suitable range of IGF-1 concertation for enhancing myogenesis, a scaffold system with hierarchical pores that is permissive to cellular infiltration/activity and controlled IGF-1 local delivery was engineered ³⁰. FIG. 21A demonstrates the microscopic structure of the scaffold. Mesoscale pores, with an average diameter of around 80+4 μm were incorporated into the inherently porous Ge1MA hydrogel using a foaming approach (FIG. 21B). A handheld high-speed stirrer (15000 RPM) was used to introduce microbubbles inside a 15% Ge1MA solution to generate a colloidal bioink (FIG. 21 Ai). Given the hydrophilicity of the hydrogel scaffold and differential densities of air and hydrogel, the bubbles quickly disrupt the thin membrane between them (FIG. 21Aii: white arrowheads are pointing to the ruptured thin membrane), and are released from the scaffold when submerged it into an aqueous environment, generating an interconnected mesoporous scaffold. These large pores are also connected through microscale hydrogel pores inherent to Ge1MA (average size ˜ 6 μm) in the other regions (FIG. 21Aiii). These data together demonstrate the capability of this simple but robust process for the fabrication of scaffolds with hierarchically interconnected pores. Quantification of different pore sizes is shown in FIG. 21B.

To incorporate IGF-1 into the above-mentioned system, gelatin microparticles were synthesized, loaded with IGF-1, and supplemented into the Ge1MA precursor. FIG. 21C shows the synthesized gelatin microparticles, with an average size of ˜ 4 μm (FIG. 21D). The size of the particles was designed to physically entrap them within the Ge1MA network and prevent their premature release with possible hydrogel movement after in vivo implantation. Furthermore, the negatively charged gelatin type B microparticles could electrostatically interact with positively charged Ge1MA scaffold synthesized from type A gelatin, and generate a higher level of affinity. The yellow arrowheads in FIG. 21Aiii show the adhered gelatin microparticles to the Ge1MA network. The IGF-1 loaded particles encapsulated in the Ge1MA network enabled a sustained release of IGF-1 from the scaffold (FIG. 21E). The microstructure of the foam increased the diffusion of IGF-1 compared to bulk Ge1MA scaffolds. While IGF-1 is a positively charged molecule, the presence of negative gelatin microparticles, linking the positive IGF-1 to positive Ge1MA, prevented a burst release of growth factor from the scaffold, and enabled the release of growth factor at physiologically relevant concentrations (>1 ng/ml per day) for multiple days (FIG. 21E).

The mechanical properties of the scaffold were further measured to evaluate the effect of mesoporosity and incorporated particles on the scaffold's stiffness (FIG. 21F), and the values with adhesion to native muscle tissue were compared (FIG. 21G). The results of compression tests demonstrated that the Young's modulus of the Ge1MA hydrogel (15% w/v) decreased significantly (p<0.0001) from 76+10 kPa for bulk Ge1MA to 8+2 kPa after foaming, and the addition of gelatin microparticles did not significantly affect the results (FIG. 21F). Interestingly, these results demonstrate that the stiffness of the foam resembles the bulk properties of native skeletal muscle (compressive modulus around 12 kPa 51), while the stiffness of Ge1MA is similar to that of native muscle fibers (compressive modulus around 50 kPa for fast and 100 kPa for slow-twitch fibers 52).

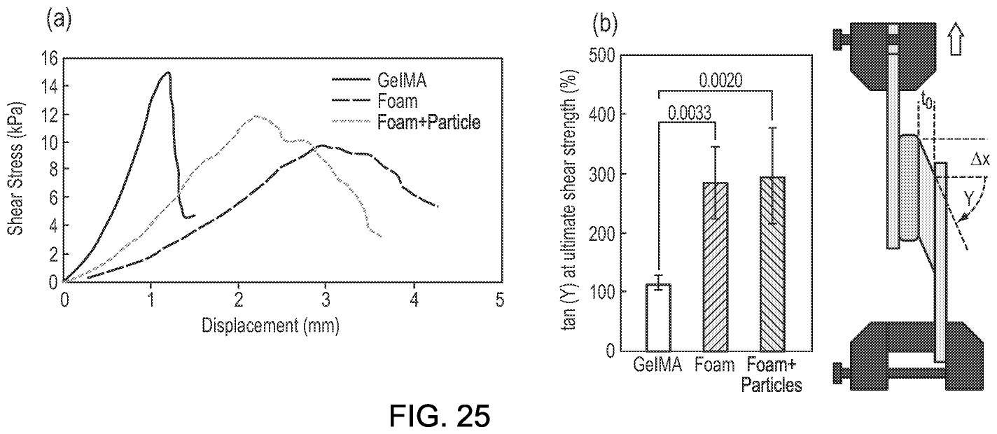

To evaluate the adhesion of the engineered biomaterials into the muscle upon in situ printing, a shear test was performed as shown schematically in FIG. 21G. The bioink was printed on the muscle tissue glued to a glass slide, a TMSPMA coated glass slide was placed on top of the printed construct, and the hydrogel was crosslinked in situ. The results show that in situ crosslinking of both Ge1MA and the foam adhere to muscle tissue (adhesion strength >9 kPa). Furthermore, the adhesion strength was reduced from 16+1 kPa for Ge1MA hydrogel to 9+2 kPa for foam (p<0.002). The less pronounced difference in adhesion strength of the foam and hydrogel in comparison to the difference in compressive moduli could be attributed to the significant deformability of the foam (FIG. 25). Furthermore, the results demonstrated that the incorporation of gelatin microparticles only slightly increased the adhesion strength of the foam to the tissue to 11+3 kPa.

Finally, the ability of the bioink to form filaments and maintain the multiscale porous structure upon deposition and crosslinking was evaluated. The printability of the foam was assessed through evaluation of the bioink status on the nozzle tip as previously described 53. FIG. 21H shows that the extruded foam from the nozzle tip had a smooth filament morphology, which confirms the printability of this bioink 53. Furthermore, the bright-field micrograph of a printed filament, shown in FIG. 211, shows the preserved mesoporous structure of the bioink after deposition, as well as a smooth and uniform filament size along the printing direction.

FIG. 21 shows the development and characterization of the engineered bioink. (A) SEM micrographs demonstrated the multiscale porous structure of the foam bioink and incorporated gelatin microparticles into the structure. (i), (ii), and (iii) show pores in different scales ((i) indicates the foam-induced mesopores, and (iii) indicates the inherent micropores). White arrowheads in (ii) show ruptured thin membranes between the bubbles, as a result of foam submersion in saline solution, forming an interconnected mesoporous structure. Yellow arrowheads indicate the adhered gelatin microparticles into the structure. (B) Quantitative assessment of different pore sizes in the engineered scaffold. (C) SEM image of gelatin microparticles after synthesis. (D) The size of gelatin microparticles measure from SEM micrographs using ImageJ software. (E) Release profile of IGF-1 growth factor from microparticles, foam, foam with microparticles, and solid Ge1MA. A sustained release of growth factor for more than a week was detected. The inclusion of microparticles in the foam scaffold slowed the release of IGF-1. (F) Compression test for evaluating the mechanical properties of the scaffolds. The test setup is shown schematically on the left, while the results are graphed as compressive modulus on the right. A significant decrease was detected upon foaming, while the incorporation of microparticles did not significantly affect the results. (G) Evaluation of scaffold adhesion capability to the tissue. A shear test (left schematic) was used to measure the adhesion of the printed scaffold to the muscle and the results were graphed as ultimate shear strength (right graph). While the adhesion significantly decreased through foaming, all of the scaffolds demonstrated strong adhesion to the tissue as a result of in situ crosslinking. (H) A smooth long filament extruded from the nozzle tip demonstrated a high level of foam bioink printability. (I) Bright-field micrograph of a printed filament showing the preserved mesoporous structure of the scaffold after printing.

### 2.3 In Situ Printing of the Engineered Scaffolds for the Treatment of VML Injury

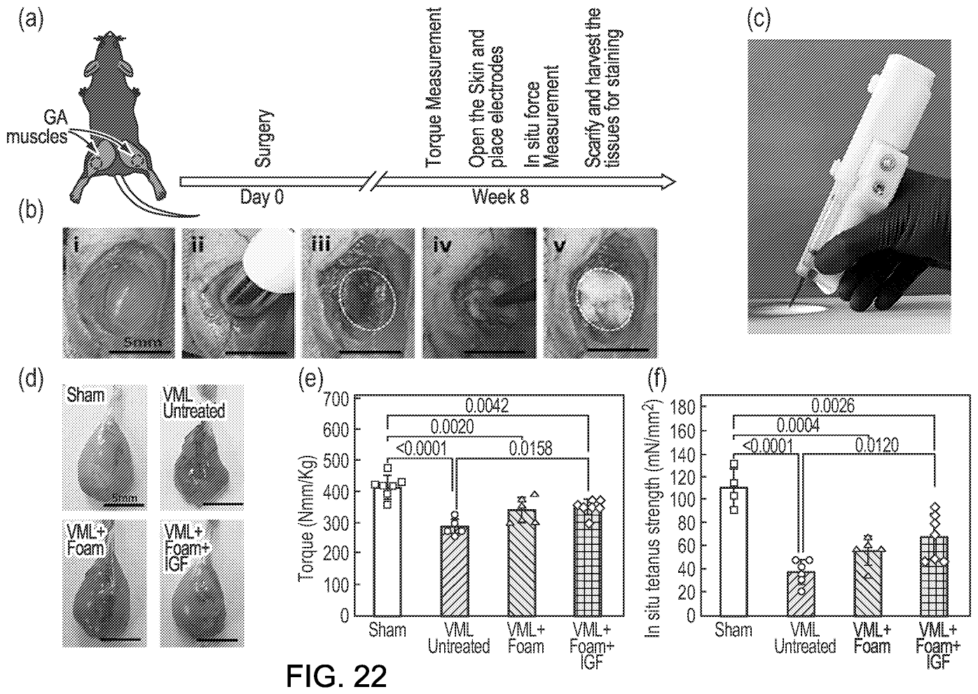

A murine model of posterior compartment VML injury of the leg, as described in the first section of the results, was implemented here to evaluate the efficacy of foam scaffold treatment of VML (FIG. 22). Four different groups were included in the study to evaluate the efficacy of the in vivo printed IGF-1-eluting scaffold with multiscale porosity on muscle regeneration: (i) a sham operation with no muscle injury, (ii) VML injury without any treatment (VML Untreated), (iii) VML injury followed by in situ printing of foam scaffold (VML+Foam), and (iv) VML injury followed by in situ printing of IGF-1-eluting foam scaffold (VML+Foam+IGF). FIG. 22A schematically shows the workflow of this animal study. On day 0, VML injury was induced in the gastrocnemius (GA) muscle of mice bilaterally, followed by treatment of the injuries according to the above-mentioned groups. For this procedure, the skin was opened (FIG. 22Bi), around 20% of the GA muscle mass was removed with a 4 mm biopsy punch (FIG. 22Bii, iii), and the engineered bioink was directly printed into the defect and simultaneously crosslinked in situ (FIG. 22Biv, v). For in situ printing, our custom handheld printer ^{24,28} with an integrated photocrosslinking mechanism was utilized here (FIG. 22C). In all of the experiments, the foam adhered securely to the remnant muscle tissue despite the wet microenvironment of the wound (FIG. 22Bv), preventing scaffold movement during the skin closure after the surgery or possible body movement. Eight weeks following the surgery, the functional recovery of the hindlimbs was assessed through in vivo torque measurement, followed by in situ twitch and tetanus force measurement of GA muscle, and finally, the muscle tissues were harvested for future histological analysis.

Gross representative images of the harvested muscle eight weeks post-surgery demonstrate the restored muscle volume in different groups (FIG. 22D). As previously shown, the regeneration in the VML Untreated group was minimum, while both VML+Foam and VML+Foam+IGF groups demonstrated a high level of regeneration. The quantitative measurements of the muscle strength agree with these gross observations (FIG. 22E, F). The results of torque measurements, which evaluated ankle plantarflexion, (FIG. 22E) demonstrated a significant (p=0.0158) recovery in the VML+Foam+IGF group (348.5±26.5 Nmm/Kg) compared to the untreated injuries (286.3±24.2 Nmm/Kg). Furthermore, the average tetanus strength of GA was significantly higher with (p=0.0120) VMLs treated with in situ printing of Foam+IGF (67.6+19.8 mN/mm²) compared to the VML Untreated group (36.9+10.5 mN/mm²).

FIG. 22 shows an application of in situ printing of engineered bioink for murine VML treatment and macroscopic evaluation of its effectiveness. (A) The workflow of animal studies in this study. On day 0, surgeries were performed through induction of VML in GA muscle of both mice legs, and treatments were applied as shown in (B). After 8 weeks, the functional recovery of the legs strength was measured using torque measurement, followed by opening the skin and direct in situ measurement of GA muscle force generation capability. The animals then were sacrificed and tissues were harvested for histology analysis. (B) The VML induction and treatment procedure. After opening the skin, the GA muscle was exposed (i) and a 4 mm biopsy punch was implemented (ii) to remove ˜20% of the muscle mass (iii). Then, the engineered bioink was directly printed in VML defect and crosslinked in situ (iv, v). (C) The custom handheld printer with an integrated photocrosslinking mechanism was used for this study. (D) Gross representative pictures of harvested GA muscle demonstrating the restored volume of the muscle eight weeks post-surgery as a result of in situ printing of engineered scaffold. (E) Assessment of functional recovery of the leg strength using torque measurements. Significant functional recovery was detected in VML+Foam+IGF group compared to untreated muscles. (F) Evaluation of GA muscle recovery after eight weeks of VML induction through in situ tetanus force measurements. A statistically significant recovery was detected in muscles treated with VML+Foam+IGF compared to untreated muscles.

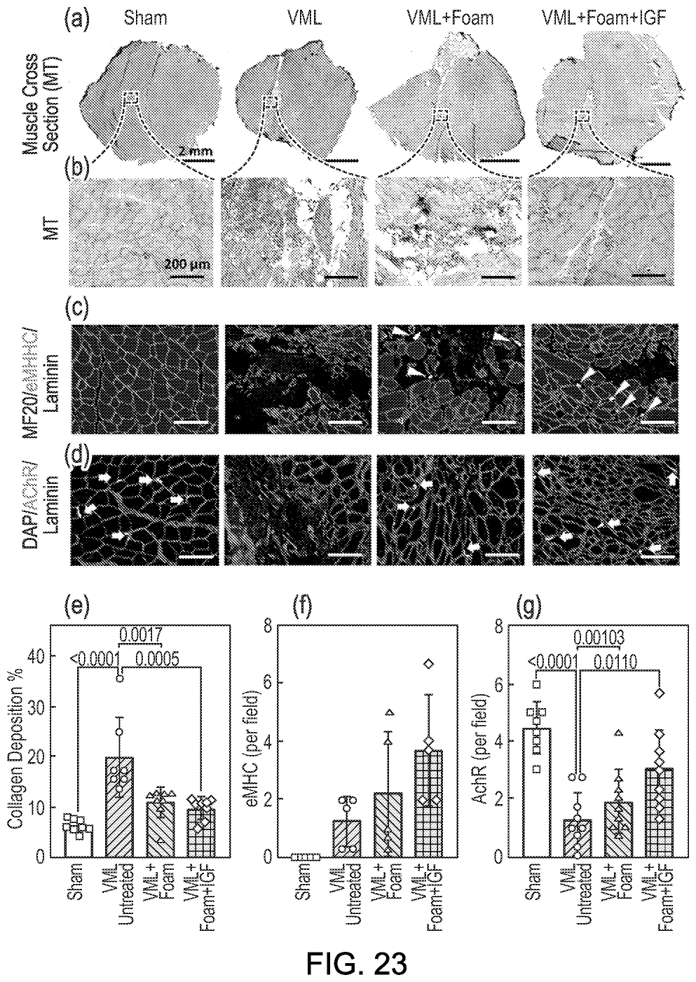

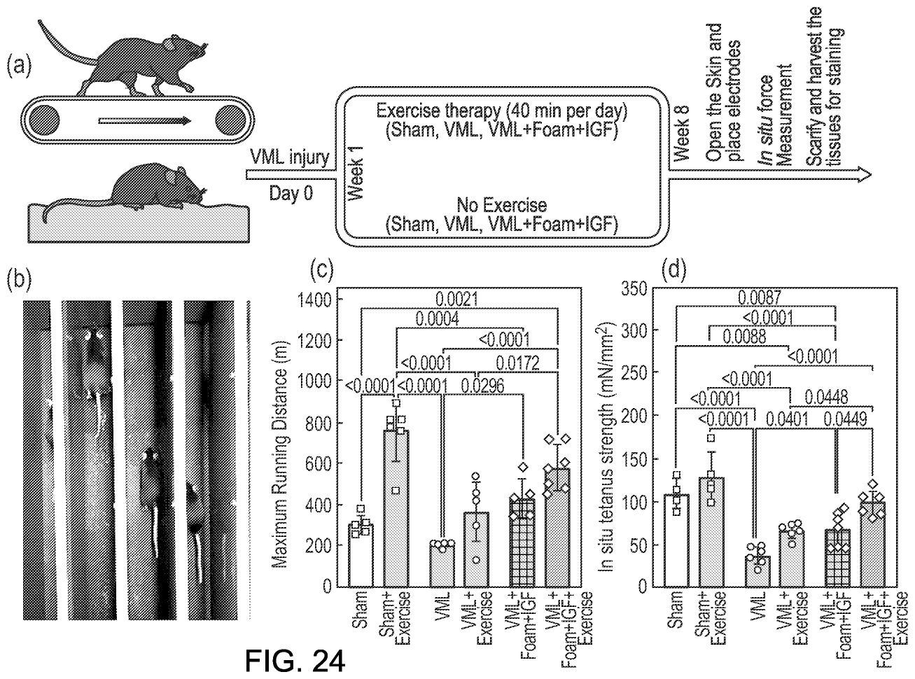

Next, histological analysis was performed to assess muscle regeneration from a microscopic perspective (FIG. 23). Masson Trichrome (MT) staining of the muscle cross section showed that while extensive fibrosis was present in the remnant muscles following untreated VML injuries, soft tissue reconstitution and limit the fibrosis were notable in both foam alone or foam supplemented with IGF-1 loaded microparticles (FIG. 23A, FIG. 23B). The regeneration of muscle fibers was further evaluated (FIG. 23C) using triple immunofluorescent staining of the basal lamina component (laminin), sarcomere myosin heavy chain that marks mature, reminant fibers (MF20), and embryonic myosin heavy chain that marks regenerating fiber (eMHC). The injury site of untreated VML showed limited signs of muscle regeneration, with no or few regenerating fibers poorly aligned to the remnant myofibers. Finally, in order to assess the level of muscle maturation and functional regeneration, triple-immunofluorescent staining for laminin, cell nuclei (DAPI), and acetylcholine receptor (AChR), a component of neuromuscular junctions (NMJs), was performed on the harvested muscle tissues (FIG. 23D). While some innervation (shown by white arrows) was indicated by the presence of a few post-synaptic AChRs in both treatment groups, the VML+Foam+IGF group seems to have the greatest density of AChRs within the injured area.

Quantitative analysis of staining signals suggests further advantages of the proposed strategy in for VML treatment (FIG. 23E-G). Measurement of collagen deposition area (FIG. 23E) demonstrated a significant reduction in the level of fibrosis in both VML+Foam (11±3%) and VML+Foam+IGF (10±2%) groups compared to untreated injuries (20+8%). Furthermore, while a statistically significant difference was not detected in the number of eMHC-expressing myofibers between different groups, a clear increasing trend toward the VML+Foam+IGF group was observed, suggesting a relatively higher level of ongoing regeneration in this group (FIG. 23F). Finally, AChRs (FIG. 23G) demonstrated a significant (p=0.011) increase in innervation when VML was treated with VML+Foam+IGF (3.0±1.4) compared to VML Untreated (1.3±1.0). It was additionally noted that there were no statistical differences in the AChR density between uninjured muscle and the muscle injured with VML treated with in situ printing of IGF-1—eluting foam.

FIG. 23 shows the microscopic evaluation of regeneration of VML injury treated with in situ printing of engineered scaffold 8 weeks post-surgery. (A) Cross sections of the muscle that were harvested from the mice and stained using the MT approach. The magnified images of the injury area are provided in (B). MT staining demonstrates a reduced level of fibrosis in both treatment groups compared to VML Untreated group. (C) Triple-immunofluorescent staining for myofibers basal lamina (Laminin), sarcomere myosin heavy chain (MF20), and embryonic myosin heavy chain (eMHC). Denser and well-oriented muscle fibers with a higher amount of eMHC signal were observed in the injury area of the VML+Foam+IGF samples compared to other groups. (D) Triple-immunofluorescent staining for Laminin, nuclei (DAPI), and acetylcholine receptor (AchR), a component of neuromuscular junctions. A higher level of AchR signal with nuclei positioned at the border of the fibers in the VML+Foam+IGF group demonstrates a high level of regeneration and muscle maturation.

### 2.4 Synergistic Effects of In Situ Printing and Excursive Therapy for VML Treatment

In order to evaluate the effects of exercise on the VML treatments, VML injuries were created bilaterally on the gastrocnemius muscles (GA) as previously described and were subsequently treated with either in situ printing of foam+IGF or no treatment. The sham group without VML injury was used as a negative control. Following three days of recovery, the animals were acclimatized to running on a treadmill for 3 days. All groups were then subjected to an 8-week-long exercise regimen comprised of running on a treadmill at 12 m/min for 40 minutes, three times weekly or no regimented exercise training program (FIG. 23A,B). At the end of 8 weeks, the maximal distance of running was measured two days following the completion of the respective courses, and functional recovery of the injured GA muscle was tested through in situ measurement of tetanus force as described in the previous section.

Following the eight-week period of regimented exercise or no additional activity, mice with sham injury exhibited the greatest capacity for distance running distance (FIG. 24C). Mice treated with foam and IGF following VML injury were able to run 30% further than mice with VML injury alone following regimented exercise training (p=0.02). In situ strength testing followed similar trends (FIG. 24D). Mice that underwent sham injury exhibited the greatest strength in their gastrocnemius muscle. Exercise improved in situ gastrocnemius strength following VML treated with foam with IGF-1 by approximately 30% (p=0.04), but this improvement was absent in mice with VML injury alone. Additionally, VML treatment with IGF-1 and exercise improved in situ gastrocnemius strength by approximately 25% in comparison to VML injury following regimented exercise (p=0.04).

FIG. 24 shows (A) A schematic of the 8-week-long exercise regimen. (B) Evaluation of the maximal distance of running. Exercise alone did not significantly improve the functional recovery of GA following VML injuries. VML+Foam+IGF+Exercise group demonstrated greater maximal running distance than VML and VML+exercise, indicating a synergistic effect of IGF foam with exercise on physiological recovery from VML. (C) Evaluation of GA functional recovery after eight weeks of VML induction through in situ tetanus force measurements. Exercise alone did not significantly improve the muscle strength, while significantly higher average muscle force was observed in the Foam+IGF as compared to the VML group. The VML+Foam+IGF+Foam group showed significantly higher muscle strength than the VML. VML+exercise, VML+Foam+IGF groups, suggesting a synergetic effect of exercise on the Foam+IGF treatment following VML.

### 3 Discussion and Conclusions

While successful treatment of VML remains challenging, tissue engineering may offer a solution to improving patient recovery. In this study, we developed a simple, but effective strategy for VML treatment that combines regenerative therapy and regimented exercise. The strategy is based on the engineering of a bioink optimized for enhancing muscle regeneration and its delivery using a highly translational and robust in vivo handheld printers. To improve the efficacy, the bioink addressed two important factors: (i) the requirement for a simple and clinically translatable preparation and application and (ii) the need for physiochemical properties permissive/promotive of myogenesis. Ge1MA was selected as the primary biomaterial constituting the bioink due to its biologically favorable structure providing cell-binding sites and biodegradable motifs, as well as its facile photocrosslinking ⁵⁴. However, standard Ge1MA, without any modification, suffers from important drawbacks: while very low concentrations of Ge1MA allow cellular migration within the 3D structure, the biofabrication and implantation of constructs made with low Ge1MA concentration are extremely challenging if not impossible ¹. Furthermore, such scaffolds degrade fast in the harsh injury environment, limiting the efficacy of the scaffold for regeneration. Alternatively, high concentrations of Ge1MA can be implemented, but this significantly reduces cellular activity within the scaffold ¹. Considering that satellite cells as well as immune cells and other cell types responsible for muscle regeneration need a 3D space supporting a high level of cellular activity and nutrient turnover, Ge1MA is reported to be inadequate for proper muscle regeneration and functional recovery ¹. To overcome this, we developed a modified version of Ge1MA through simple stirring to incorporate mesoscale porosity into its structure.

The mechanical stirring initially introduces air inside the Ge1MA solution, followed by shear-induced bubble splitting that forms microbubbles ⁵⁵. While the protein nature of the Ge1MA can act as a surfactant and stabilize the generated microbubbles ⁵⁶, PVA is used in the formulation to further prevent bubble merging and enable the formation of a stable colloidal bioink. Upon crosslinking, a multiscale porous structure comprising foam-induced mesopores and inherent Ge1MA micropores is generated. Due to the proximity of the bubbles in the colloidal solution, a very thin membrane is formed between the pores where the bubbles were contacting, making the structure susceptible to rupture and formation of interconnected mesoporous morphology. Upon submersion into an aqueous solution, the thin membranes break with a driving force to merge and release bubble from the structure to reduce the interfacial and gravitational energy of the system. The interconnected porous structure through both mesoscale and microscale pores provides an ideal environment for cellular activity as well as nutrient transport ^{57,58}.

The multiscale porous also offers multiscale biomimetic mechanical properties for better tissue integration, myogenesis, and muscle function recovery ^{51,59}. A 15% Ge1MA solution was selected as the foam precursor to recapitulate the desired biomimetic mechanical properties. From a macroscale perspective, the elastic modulus of the foam is very close to that of bulk skeletal muscle (around 8 kPa for foam vs 8-17 kPa reported for skeletal muscle 51). On a microscale view, the elastic modulus of the Ge1MA regions between the bubbles resembles the elastic modulus of individual muscle fibers (around 80 kPa for Ge1MA hydrogel vs 50 and 100 kPa for fast and slow-twitch fibers 52).

In addition, foaming also enhanced the deformability of the scaffold. Since muscles contract, a highly deformable implant capable of complying with large strains is desirable ⁶⁰. The foam scaffold demonstrated close to 300% deformability in shear tests, making it an ideal candidate for skeletal muscle tissue engineering. Furthermore, the shear tests showed a strong adhesion of the scaffold to the muscle tissue. Secure adhesion of the implants to the tissue ensures minimum displacement of the implant during the surgery or as a result of body movement post surgery, enhancing the likelihood of implant-tissue integration ²⁴. Ge1MA hydrogel has been reported to establish strong adhesion to the tissue upon in situ crosslinking due to the physical interlocking, the formation of covalent bonds upon generation of free radicals during photocrosslinking, and hydrogen bonds between free hydroxyl groups in the Ge1MA structure and the tissue ^{24,61,62}. Our results were in agreement with the previous findings. However, Ge1MA foams were more flexible and therefore the adhesion strength of Ge1MA foam was comparable to the values of Ge1MA hydrogel.

To integrate biochemical cues into the foam scaffold to enhance regenerative response, IGF-1 was incorporated into the scaffold. IGF-1 is an important mediator of growth hormone (GH) in promoting muscle growth and it is known to exert anabolic effects on muscle. Here, we demonstrated that IGF-1 could enhance the proliferation rate of muscle progenitors. An increased myoblast density significantly affects their behavior through enhanced cell-cell communication. Previous reports demonstrated that increased myoblast density can promote their alignment ³⁹, which is important for their differentiation and final muscle fiber functionality ⁴⁶. A better-aligned organization was observed in the myoblasts cultured with a physiologically relevant IGF-1 level. Not only through enhanced myoblast density but also independently, IGF-1 enhances myogenesis and muscle maturation ^{8,63}. Gene expression analysis demonstrated that IGF-1 significantly augmented late-stage myogenic gene expression indicating enhanced myoblast differentiation, as well as genes associated with functional force-generating fibers. In addition, genes expression of ECM and cell adhesion proteins were significantly elevated, indicating better cell-cell and cell-ECM communication. However, as shown by our result, the level of IGF-1 in the remnant muscle is significantly reduced upon VML injury: this necessitates supplementation of IGF-1 in the defect area with sustained release of exogenous IGF-1 from implanted scaffolds. Importantly, localized, targeted application of IGF-1 has an important biological advantage in that it can mitigate any potential adverse effects of increasing the systemic level of IGF-1 and manipulating the GH-axis, such as hypoglycemia and reduced GH release ^{64,65}. Since IGF-1 is positively charged ⁴⁹ a strategy was developed to avoid its burst release from a positively charged GetMA foam network due to repulsive electrostatic interactions (Ge1MA is made from positively charged gelatin type A 48). Negatively charged gelatin microparticles were used as the carrier of a positively charges molecule. Microparticles were was first loaded with IGF-1 and then encapsulated into the foam structure.

The composite bioink was tested for its printability before implementing on a murine model for VML. When printing a gelatin-based material, partial thermal gelation is advantageous before extrusion of the material through the nozzle to prevent under-gelation or over-gelation that causes poor printability ⁵³. However, controlling thermal gelation of Ge1MA and therefore its 3D printing is extremely challenging and the bioink often suffers from under-gelation or over-gelation ⁵³. In contrast, we found the foam bioink highly printable. This is due to its rapid sol-gel transition. Upon extrusion, the foam solution immediately solidifies as a result of thermal gelation, forming a smooth filament at the nozzle tip as shown by our results, even if the bioink is at temperatures above sol-gel transition and therefore is completely in solution phase inside the syringe. The more rapid sol-gel transition of foam compared to unmodified Ge1MA is due to the low density of the foam bioink, making its surface-area-to-volume ratio higher, accelerating the temperature change. It is noteworthy that an acellular bioink was developed in this study to provide an effective but simple and clinically translatable approach. While cellular scaffolds have been implemented widely in muscle tissue engineering, the application of the cells makes the process much more complex and the regulatory pathway toward clinical translation more cumbersome 1. Furthermore, the testing of cellular scaffolds requires the use of immunocompromised animals, which can skew the obtained results given the critical role of the immune system in muscle regeneration ^{8,66}. An additional benefit of acellular scaffolds over cellular scaffolds is a faster response in the treatment of injury, which is critical in clinical settings.

The developed strategy was tested for the treatment of VML injury in wild-type

mice. To demonstrate the benefit of our IGF-1 impregnated foam scaffolds, we utilized a validated murine model of VML injury in which en bloc resection of the gastrocnemius muscle in the posterior compartment is performed ^{17,30,67}. This type of injury results in loss of both skeletal muscle strength and functional capacity, as measured by running endurance. Our results suggest that placement of an IGF-1 foam scaffold within an acute VML injury improves both skeletal muscle strength and functional recovery and, by eight weeks following injury, the functional status approaches healthy, uninjured muscle. In addition, this improvement in muscle function is accompanied by a decrease in fibrosis and evidence of de novo skeletal muscle regeneration within the scaffold itself. The recovery in muscle function demonstrated here is similar to others who have employed the use of cells 45.

Others have demonstrated improvements in VML recovery following scaffold placement, including VML treatment with porcine urinary bladder extracellular matrix ³⁶or collagen glycosaminoglycan matrixes ³². Importantly, a recent meta-analysis suggested that functional strength improvement was only marginal with the use of acellular scaffold therapy (16%) in comparison with no treatment at all 68. Separately, decellularized scaffolds, hydrogels, nanofibers, and electroconductive scaffolds have all been evaluated, with equivocal benefit ⁶⁹. The modest functional improvement provided correlates to mixed results on the ability of ECMs to promote de novo skeletal muscle regeneration ⁷⁰. Our previous studies suggest a small improvement in functional muscle recovery following VML with the use of Ge1MA or collagen GAG scaffolds, but with minimal tissue ingrowth within the scaffold itself ^{28,71}. The use of a foam scaffold, however, is distinct in the amount of tissue growth within the scaffold itself and a paucity of fibrosis.

Physical therapies are an important part of rehabilitation following muscle injuries ⁶⁸. Exercise-based therapies remain to be the most commonly prescribed, clinically proven methods for promoting functional recovery of muscular injuries, and some evidence suggests its benefit on muscle regeneration following injuries ^{17,68,72}, although with limited efficacy. In order to better simulate the clinical setting and to test the effect of exercise on the foam-based treatment for VML recovery, VML treatment was followed by regimented exercise. VML treated with Foam+IGF scaffold demonstrated significantly higher in situ force production when it was also exercised, and the group additionally outperformed the VML only and Foam only groups in both the maximal running distance and force production, indicating a synergetic effect of those two treatments. The exact mechanism(s) by which exercise enhances the therapeutic effects of the IGF foam remains to be elucidated, but several or a combination of the following may describe plausible interplays between the two treatments.

Activity-induced muscular adaptations rely mostly on local (as opposed to systemic) production of growth factors in response to mechanosensory stimulation of muscle contraction within skeletal muscle. Those factors in turn exert anabolic effects on the exercised muscle in a paracrine fashion ⁷³. Loss of muscle mass in VML would result in a significant loss of this exercise-induced IGF production, and this IGF-releasing scaffold can supplement this precisely where such loss has occurred through providing both IGF locally and possibly a point of mechano-sensory cues to induce regenerative response during exercise. Whilst IGF-1 has been shown to have direct anabolic effects on skeletal muscle, its effect is determined by the levels of IGF-binding proteins. Indeed, exercise-induced proteolysis of IGF-binding protein-3 (IGFBP-3) may have contributed to the enhanced anabolic effects of the IGF-releasing scaffold by increasing the bioavailability of IGF-1 at the site of injury ⁷⁴. In addition, exercise has been shown to augment circulation, angiogenesis, and hasten (re-)innervation of muscles following injuries^{10,75,76}, and these likely facilitate faster functional recovery. Overall, combined rehabilitation and regeneration therapy with IGF-releasing scaffold seems to work synergistically, and the observed functional benefits demonstrate a promising prospect for its clinical application.

In conclusion, while a variety of tissue engineering strategies have been proposed in prior studies for implantation into the muscle defects with some promise in limiting fibrosis and improving hypertrophy of remnant muscle^{1,18}, very few could achieve results similar to this work. Furthermore, the translation capability of those strategies is limited by their scalability, considering the poor oxygen and nutrient diffusion, and consequently cellular activity, in large scaffolds usually required for clinical treatment of VML ^{38,39}. In contrast, in situ printing of the highly porous foam scaffold proposed in this study, resulted in the restoration of muscle fibers, tissue maturation as indicated by innervation and muscle fiber nuclei position. Furthermore, the strategy used here is very simple, translatable, and effective, which can be attributed to the higher capability of cell infiltration inside the foam as a result of its higher porosities and homogeneous interconnected pore network, along with the sustained release of IGF-1.

### 4 Materials and Methods

### 4.1 Materials

Recombinant Mouse IGF-1 was obtained from R&D Systems (MN, USA). Cell culture reagents including Dulbecco's phosphate-buffered saline (DPBS), Dulbecco's modified eagle medium (DMEM), fetal bovine serum (FBS), penicillin-streptomycin (PS), trypsin-ethylenediaminetetraacetic acid (trypsin-EDTA), horse serum (HS) and 4-(2-hydroxyethyl)-1-piperazineethanesulfonic acid (HEPES) were purchased from Thermofisher Scientific (Gibco, MA, USA). For characterization of in vitro cell studies, PrestoBlue™ reagent (Invitrogen), Alexa Fluor 488 Phalloidin, and DAPI were obtained from Thermofisher Scientific, while Triton X-100 and bovine serum albumin (BSA) were purchased from Sigma-Aldrich (MO, USA). Real-time quantitative PCR (RT-qPCR) reagents including TRIzol (Invitrogen), and SuperScript III First-Strand Synthesis SuperMix (Invitrogen) were purchased from Thermofisher Scientific, while iTaq Universal SYBR Green Supermix was obtained from Bio-Rad (CA, USA).

Ge1MA with a medium degree of methacrylation was synthesized based on an established protocol. Gelatin from porcine skin, type A, with a 300 g Bloom (Sigma-Aldrich) was dissolved in DPBS at a 10% concentration under 240 rpm stirring at 50° C. for 1 h. Methacrylic anhydride was then added dropwise to the solution at a 1.25% (v/v) concentration, followed by its incubation under vigorous stirring (500 rpm) for 1h. To stop the reaction, the solution was diluted with DPBS eight times and stirred at 240 rpm and 50° C. for 10 min. The solution was then transferred into dialysis tubing with 12-14 kDa cutoff pore size (Spectrum, Fisher Scientific) and dialyzed against DI water for a week at 40° ° C. by changing the water twice a day. Finally, the solution was filtered using Steritop vacuum filters (Sigma-Aldrich), frozen at −80° ° C. for two days, and lyophilized for a week in a FreeZoneR benchtop freeze dryer (LabconcoR), MO, USA) to obtain dried Ge1MA. Ge1MA was stored at −20° C. until use.

Gelatin microparticles were synthesized using a desolvation approach. A 5% (w/v) type B gelatin from bovine skin (225 g Bloom, Sigma-Aldrich) was prepared by stirring at 200 rpm and 50° C. Then, acetone (Sigma-Aldrich) was added to the solution at a 1:1 volumetric ratio at room temperature and the supernatant was discarded. The precipitate was redissolved in DI water to recover the volume of the solution and the pH was adjusted to 12 by the addition of 3 mol/L NaOH (Sigma-Aldrich). Acetone was then added dropwise at a 3:1 volumetric ratio (acetone: gelatin solution), and the solution was shaken for 10 min, followed by the addition of glutaraldehyde (25% solution, Sigma-Aldrich) at a 0.25% (v/v) concentration. The solution was stirred overnight at 50° ° C., and 200 rpm and particles were harvested by triple centrifugation (10,000 g for 30 min) and redispersion in a 100% ethanol solution. The particles were subsequently freeze-dried for 12 h and stored at −20° C. until use.

4.2 Cell culture

C2C12 mouse myoblast cells were subcultured up to passage 10 by detaching the cells using Trypsin-EDTA and resuspending them in a growth medium containing DMEM supplemented with 10% FBS and 1% PS. For the main experiments, the cells were cultured for 7 days in the growth media containing IGF-1 at 0 ng/ml, 1 ng/ml, or 10 ng/ml concentrations. After 7 days, the culture medium was replaced with a differentiation medium, composed of DMEM supplemented with 2% HS, 20 mM HEPES, and 1% PS. C2C12 cells were cultured in the differentiation medium containing 0 ng/ml, 1 ng/ml, or 10 ng/ml IGF-I for an additional 14 days of differentiation.

### 4.3 Evaluating the Proliferation Rate and Morphology of the Muscle Progenitors

The cell proliferation rate and activity were determined during the culture time by incubating the cells in a solution composed of 10% (v/v) PrestoBlue™M reagent in the culture media. Cultures were placed in an incubator at 37° C., and 5% CO2 for 1.5 hours. 100 μL of the supernatant was then transferred to a 96 well plate. The fluorescence intensity was detected by a multimodal plate reader (BioTek Instruments Inc., VT, US) at an excitation wavelength of 560 nm and an emission of 590 nm.

Cellular morphology was assessed using F-Actin/DAPI staining. Samples were fixed by applying a 4% (w/v) paraformaldehyde solution for 30 min, followed by three washing steps with DPBS. The cells were then treated with 0.3% (v/v) Triton-X 100 in DPBS for 10 min and washed twice with DPBS. Samples were incubated in a 1% (w/v) BSA solution in DPBS for 30 min. Subsequently, Phalloidin with a dilution of 1:40 in DPBS was incubated with cells for 40 min at room temperature in dark. Cultures were washed again and then incubated in a 1:500 diluted DAPI solution in DPBS for 10 minutes. After a final washing step, cells were visualized under a fluorescence microscope (AxioCam MRc5, Carl Zeiss, Germany).

4.4 Assessment of myogenic differentiation using RT-qPCR

In order to investigate myoblast differentiation, the expression of relevant genes was measured by RT-qPCR. TRIzol was used to extract the RNA and NanoDrop (Thermofisher Scientific) was implemented to evaluate the total RNA yield. According to the manufacturer's instructions, 1 μg of the total RNA of each sample was reverse-transcribed by using the SuperScript III First-Strand Synthesis SuperMix. At this stage, RT-PCR was performed by introducing the SYBR Green Master Mix. A 20 μL volume reaction component was prepared by mixing 10 μL of Master Mix with 1 μL of forward and reverse primers and 100 ng of cDNA template, while nuclease-free water was used to adjust to the final volume. Relative gene expressions were calculated using a AACt method, through normalizing to GAPDH gene expression.

### 4.5 Bioink Preparation

In all of the remaining studies, the bioink was consisted of 15% (w/v) Ge1MA, 1% (w/v) PVA, 1500 ng/ml IGF-1, 6 mg/ml gelatin microparticles, and 0.3% (w/v) lithium phenyl-2,4,6-trimethylbenzoylphosphinate (LAP, Sigma-Aldrich) as the photoinitiator. IGF-1 stock solution was prepared by reconstitution in DPBS containing 0.1% BSA. Gelatin microparticles were first loaded with IGF-1 by vortex mixing of 50 mg microparticles in 8000 ng/ml IGF-1 solution at 4° C. overnight. 120 μL of the mixture was then added to 1 mL of Ge1MA solution, containing LAP and PVA, and vortex mixed for 20 sec to achieve the target concentrations.

To make the foam bioink, the biocomposite was added into a syringe barrel and foamed in situ by inserting the probe of a handheld homogenizer (Bio-Gen PRO200, ProScientific, CT, USA) inside the solution and stirring it for 40 sec at 15000 rpm. For crosslinking, a 1 watt 395 nm wavelength blue light was used either implementing the integrated LED into the handheld printer or an LED flashlight.

### 4.6 SEM Analysis

SEM was performed to investigate the internal microstructure of the printed foam scaffolds. The bioink was printed on the glass slides and photo-crosslinked. The scaffolds were submerged into liquid nitrogen to immediately freeze the hydrogel and subsequently lyophilized for 24 h. The scaffolds were then broken to expose the cross-section and were coated with a thin gold layer using a sputter coater device (Vacuum Desk V, TX, USA) set at 60 sec and 20 mA. The samples were imaged using a benchtop SEM device (TM-1000, HITACHI) and images were analyzed using ImageJ software. The average pore or particle sizes obtained from at least three different samples were considered as different replicates.

### 4.7 Assessment of Release Kinetics

To evaluate the release kinetics of IGF-1 from gelatin microparticles, Ge1MA, foam, and foam with microparticle scaffolds, 100 μL of the bioink was printed into each well of a 48-well cell culture plate and crosslinked in situ as described before (η=4). The gelatin microparticle group that was not encapsulated in a scaffold was added in 100 μL of DPBS. 250 μL DPBS solution was then added on top of the samples and the plate was sealed and incubated at 37° C. At each time point, the supernatant was completely removed into a micro-centrifuge tube and replaced with fresh DPBS. The supernatants were stored at −20° C. before measuring the IGF-1 concentration. The IGF-1 concentrations were measured using a murine IGF-1 DuoSet ELISA kit (R&D Systems, MA, USA) based on the manufacturer's protocol.

### 4.8 Measurement of Mechanical Properties

Compression and lap shear tests were performed to evaluate the mechanical properties of the scaffolds in this work. An Instron 5542 mechanical tester (MA, USA) was used to perform the experiments. To perform the compression tests, the scaffolds were fabricated in cylindrical shape using a polydimethylsiloxane (PDMS: Dow, MI, USA) mold with 6 mm diameter and 5 mm height. To avoid overfilling, a glass slide was placed on top of the filled mold and the bioink was photocrosslinked through a glass slide as described before. The sample was then removed from the mold and placed between the compression plates of the device as shown in FIG. 21. A compression rate of 1 mm/min was then applied and the compression modulus was calculated from the slope of a fitted line interpolating the stress-strain data up to 10% strain. The lap shear tests were performed based on ASTM F2255-05 standard 77. Rectangular pieces of porcine muscle (13 mm×10 mm) were cut and glued into glass slides using cyanoacrylate adhesive. The bioink was then printed onto the tissue with 13 mm×10 mm×2 mm dimensions, covered with 3-(trimethoxysilyl) propyl methacrylate (TMSPMA: Sigma-Aldrich) coated glass slide, and photocrosslinked as described. The samples were subsequently secured on the mechanical testing device using grips (FIG. 21) and pulled in shear at a rate of 1 mm/min until failure occurred.

### 4.9 Handheld Printer and Printing Experiments

The handheld printer was developed as previously described ^{24,28,29}. An extrusion system was custom designed to transmit rotation from an electric micromotor (Pololu) through a linear guide rail system utilizing rolling bearings and precision shafts to a syringe filled with the bioink. Electronic control systems were also designed to control the motor power, speed, and direction as well as to activate the photocrosslinking system. The photocrosslinking system utilized a 1 W Blue LED (395 nm, CH_Town Electronic). The system was powered by a 2500 mAh battery (GTF) that was charged from a PowerBoost 1000 Charger (Adafruit). A customized housing was designed using SolidWorks (Dassault Systèmes) to enclose all systems while minimizing the device footprint and maximizing ergonomics during operator printing. The housing was 3D printed using stereolithography in an Objet260 Connex3™ (Stratasys). All components were wired and assembled by hand. During the device operation, the syringe full of the bulk or foamed Ge1MA bioink was loaded into the device, extruded into the defect size at the appropriate flow rates through a conical 22 gauge nozzle 24, and crosslinked using the blue light.

### 4.10 Animal Studies

### 4.11 Animals

All animal procedures were approved by the Institutional Animal Care and Use Committee of Brigham and Women's Hospital and were performed in compliance with the U.S. National Institutes of Health guidelines. C57BL/6 mice (10-12 weeks of age) were obtained from Jackson Laboratories. Animals were housed at the Brigham and Women's Hospital Animal Care Facility and were given ad libitum access to food and water following a 12 light/12 dark cycle. An equal number of male and female gender mice were utilized in experiments.

### 4.11.1 VML Injuries

VML was created on the gastrocnemius muscle bilaterally as follows: Under general anesthesia, depilation of the legs was performed using a clipper and razor. After sterilization with a chlorhexidine wipe, a skin incision was made along the posterior compartment of the hindlimb followed by dissection of the fascia to fully expose the underlying gastrocnemius muscle. Using a 4 mm biopsy punch, a full thinness muscle defect was created in the mid-section of the gastrocnemius muscle without separating the muscle. The defect was then filled completely with either bioprinting of the foam or foam+IGF or without any treatment. The skin and fascia incisions were then closed with simple sutures (4-0 Silk, Ethicon, Johnson Johnson, Somerville, NJ, USA). The sham group received the skin opening/closure without muscle injury. Animals were allowed to heal in their respectable cages, with the freedom to access food, and water and move around in the cage. After 8 weeks, animals were subjected to muscle strength measurements (torque measurements and in situ force measurements, as described below), and euthanized to harvest the injured muscles for histological evaluation.

### 4.11.2 Torque Measurements

Torque produced by the plantar flexor muscle of the lower limb was measured 8 weeks following the muscle injuries. Under general anesthesia, the animal's foot was secured to the footplate using adhesive tape. The tibia was aligned so that it is perpendicular to the lever. The muscle group is stimulated by placing the EMG electrodes subcutaneously to stimulate the sciatic nerve. Using the device program (610A Dynamic Muscle Control LabBook v6, Aurora Scientific, Aurora, Ontario, Canada), the current and resting tension were adjusted until maximum twitch force was produced by a single pulse with a pulse width of 0.2 ms. Torque is measured as the force measured during tetanus at this optimized setting, normalized to one of the body weights of the animals (g).

### 4.11.3 In Situ Force Measurements

In situ force tests were performed 8 weeks following muscle injury. Under general anesthesia, the skin and fascia were incised to expose the right GA muscle. The Achilles tendon was then severed at its distal end and sutured onto the lever arm of the force transducer. The soleus muscle was dissected from the tendon and removed. To stabilize the leg in position, a needle was inserted directly through the knee and the needle was then locked in place. The exposed muscle was kept moist using Ringer solution. The GA muscle was stimulated by needle electrodes placed directly on the corresponding by resting the electrodes directly on the muscle. Using the device program (610A Dynamic Muscle Control LabBook v6, Aurora Scientific, Aurora, Ontario, Canada), the current and resting tension were adjusted until maximum twitch force was produced by a single pulse with a pulse width of 0.2 ms. The optimal length (Lo) in which muscle could produce its largest force was measured as the distance between the knee and distal insertion of muscle to the tendon. Under the optimal resting tension, the tetanic force was measured with pulses given at 100 Hz with increasing amperage from 10 mA to 1A. The maximum twitch and tetanic forces of the right GA muscle of each animal were normalized to the estimated cross-sectional area (CSA) of the muscle (mm²) calculated as CSA=Muscle weight (mg)/[1.06×Lo(mm)].

### 4.12 Exercise Therapy

In order to evaluate the effects of exercise on the VML treatments, VML injuries were inflicted on the gastrocnemius muscles of mice bilaterally as previously described. The VML was subsequently treated with foam+IGF or no treatment. Following 3 days of recovery, the animals were acclimatized to running on a treadmill for 3 days, and then subjected to an 8-week-long running exercise regimen on the treadmill at 12 m/min for 40 minutes, three times weekly or no regimented exercise training program. They were allowed to move freely within their cages for the remainder of the time and were given free access to food and water. The animals were trained at the same time each time at 8 PM. At the end of 8 weeks, the maximal distance of running was measured. Briefly, the maximal distance of running was measured as the distance run by the animal at 15 m/s before it definitively stopped running, where it was assumed to be the point of exhaustion. 2 days following the physiological testing above, functional recovery of the muscle was tested through in situ measurement of GA muscle force, as described in the previous section.

### 4.13 Histological Staining and Quantifications

The cryostat muscle cross-sections were stained for Hematoxylin and Eosin (HE) and Masson trichrome (MT) using standard techniques. For immunostaining, briefly, frozen sections were thawed at room temperature for 10-20 minutes. The slides were washed twice in phosphate-buffered saline (PBS) and then incubated for 5 min in 0.05% TX-100 in PBS for permeabilization. The slides were then washed again in PBS and incubated at room temperature for 1h in a blocking solution containing 1% BSA and 5% Goat normal serum in TBS, followed by overnight incubation at 4° C. with primary antibodies (supplementary table 1) diluted in blocking buffer. Samples were washed three times in PBS and then incubated for 1 h at room temperature with secondary antibodies (supplementary table 1). The slides were then washed twice with PBS and incubated in DAPI solution for nuclei staining for 5 min. After washing the slides several times with PBS, they were mounted with ProLong™ Diamond Antifade Mountant (Invitrogen™) and glass coverslips.

Olympus model BX53 microscope (UCMAD3, T7, Japan) was used to capture histological images and ImageJ (version 1.52a: Media Cybernetics, Rockville, MD, USA) was used for image analysis and quantifications. Briefly, for collagen deposition (fibrosis) quantification, the color deconvolution and image thresholding plugins of ImageJ were used to analyze the blue area in five high power field photos (HPF) of MT-stained slides in the injury site of each muscle cross-section. To quantify embryonic myofibers, a number of eMHC positive myofibers were measured manually in three HPF images of the regenerating site of each muscle cross-section. Similarly, the number of AchRs was quantified manually to measure the number of NMJs in the regenerating area of the muscle.

### 4.14 Statistical Analysis

All of the data were presented as mean±standard deviation. The statistical analyses were performed using GraphPad Prism 9.0 software (CA, USA). One or two-way analyses of variance (ANOVA) were used in this work to compare the groups and p-values smaller than 0.05 were considered significant and shown in the graphs.

### Example 4

Various exemplary features of the disclosed compositions, systems, and methods are further described in this example.

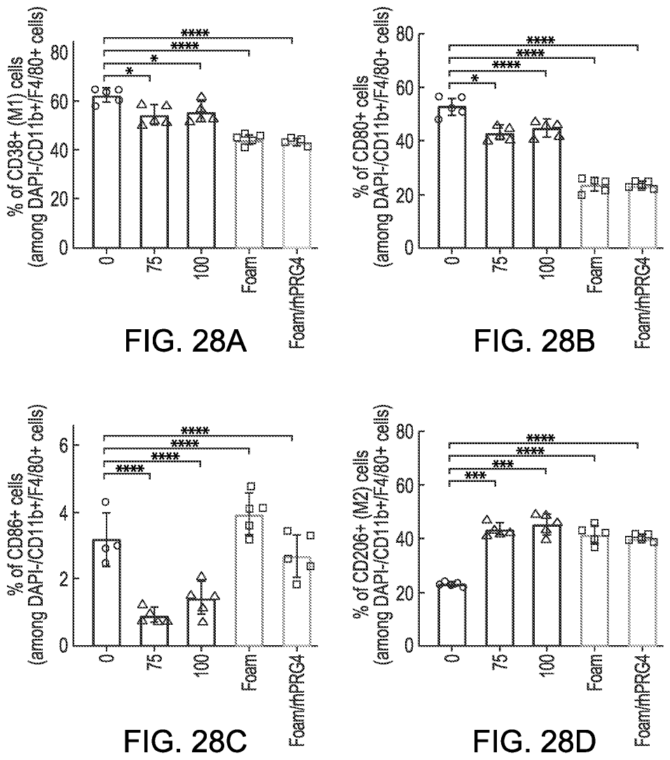

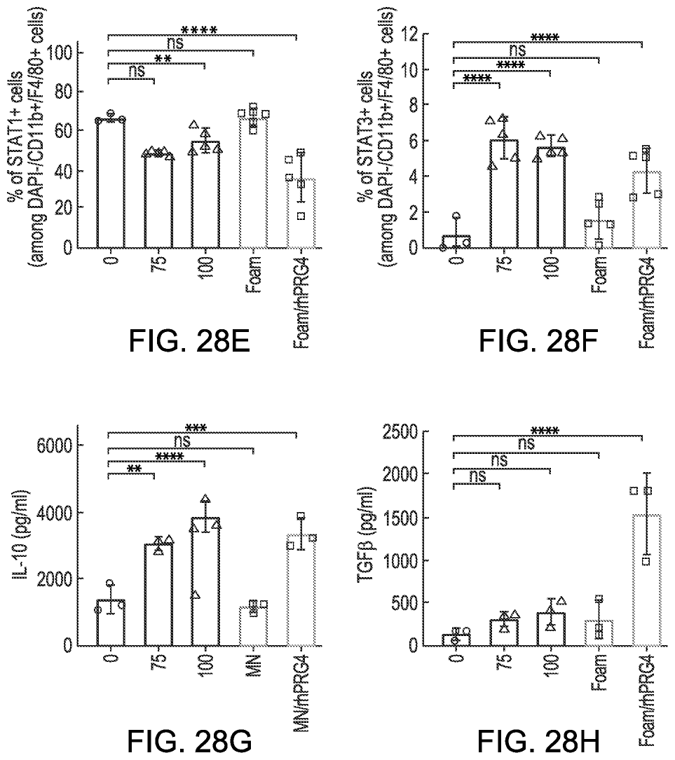

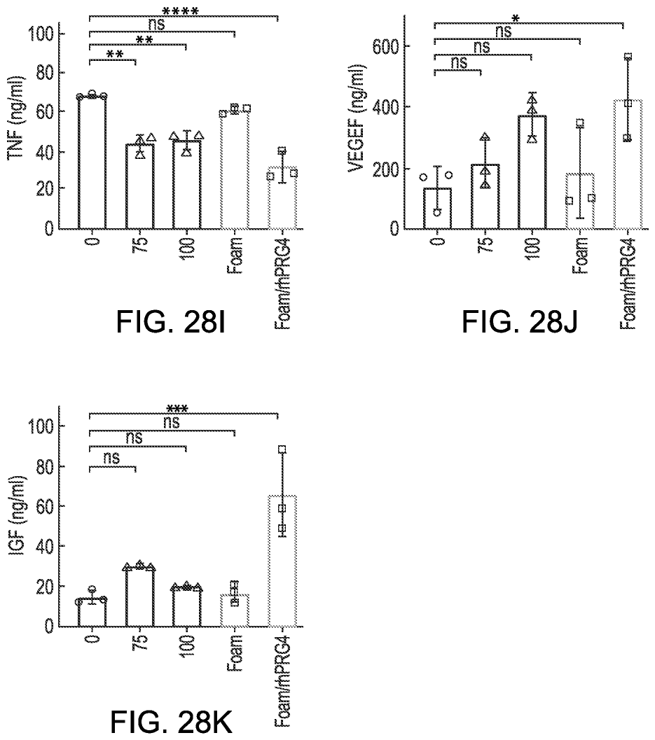

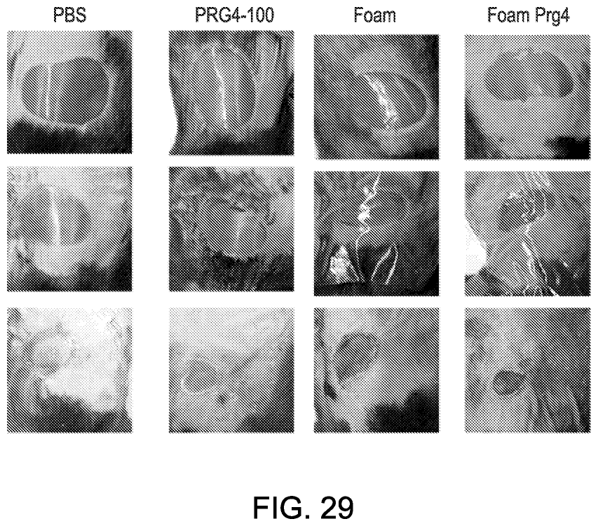

FIGS. 28(A)-28(K), 29, and 31 show the effect of exemplary Ge1MA foam releasing recombinant human proteoglycan 4 (rhPRG4) for immunoengineering. In particular, referring to FIG. 29, four different patches were tested on respective animal subjects: a non-foam patch with phosphate-buffered saline (PBS), a non-foam patch with PRG4, a foam patch, and foam patch with PRG4. FIG. 29 shows healing of the wound over time (moving down the page) for each patch type. FIGS. 28(A)-28(F) demonstrate the effect of application of this system on macrophage polarization compared to topical administration of rhPRG4 at different concentrations. FIGS. 28(G)-28(K) demonstrate the release of different growth factors by macrophages as a result of exposure to the system. Accordingly, FIGS. 28(A)-28(K) show that rhPRG4, an anti-inflammatory compound, retains its activity once encapsulated within the foam and lowers the pro-inflammatory function of activated macrophages. FIG. 31 shows percentage of the wound remaining over a 11-day span for the different patch samples. As can be seen, foam with rhPRG4 showed the greatest reduction in wound remaining at 4, 7, and 11 days.

### Exemplary Aspects

In view of the described products, systems, and methods and variations thereof, herein below are described certain more particularly described aspects of the invention. These particularly recited aspects should not however be interpreted to have any limiting effect on any different claims containing different or more general teachings described herein, or that the “particular” aspects are somehow limited in some way other than the inherent meanings of the language literally used therein.

Aspect 1: A foam biomaterial comprising:

a biomaterial ink having an interconnected pore structure, wherein the foam biomaterial has a porosity from about 10% to about 90%.

Aspect 2: The foam biomaterial of aspect 1, where the interconnected pore structure is formed without use of sacrificial materials.

Aspect 3: The foam biomaterial of aspect 1 or aspect 2, comprising at least one of: a polymer, a protein, or a combination thereof.

Aspect 4: The foam biomaterial of any one of aspects 1-3, further comprising at least one additive, the at least one additive comprising nanoparticles, microparticles, nanofibers, microfibers, an antibacterial compound, an antibiotic, a bioceramic, a ceramic, oxygen-generating material, at least one vitamin, at least one lipid, at least one phospholipid, at least one fatty acid, a biological factor, a polysaccharide, a nucleic acid, a growth factor, hydroxyapatite, calcium phosphate, dopamine-based material, carbon nanotubes, a Quaternary ammonium compound, polyhexamethylene biguanide (PHMB), methacry loyloxydodecylpyridinium bromide (MDPB), graphene, graphene oxide, a carbon derived material, liquid crystals, peptides, chitosan, silver-based materials, platelet-rich plasma, a blood-derived material, or a combination thereof.

Aspect 5: The foam biomaterial of aspect 4, wherein the additive is provided in a concentration from 0 to about 90% weight percent.

Aspect 6: The foam biomaterial of any one of aspects 1-5, where the foam biomaterial is crosslinked or solidified through physical, ionic, thermal, chemical, enzymatic, photo crosslinking, or a combination thereof.

Aspect 7: The foam biomaterial of any one of aspects 1-6, where the foam biomaterial is adhered to surrounding tissue, a medical device, or an implanting materials.

Aspect 8: The foam biomaterial of any one of aspects 1-7, wherein the interconnected pore structure supports cellular infiltration, tissue remodeling, tissue regeneration, and/or tissue fidelity and stability.

Aspect 9: The foam biomaterial of any one of aspects 1-8, wherein the foam biomaterial has a Young's modulus from 1 kPa to 100 MPa.

Aspect 10: The foam biomaterial of any one of aspects 1-9, wherein the foam biomaterial is capable of sustaining 50% strain for 10 cycles without breaking.

Aspect 11: The foam biomaterial of any one of the preceding aspects, wherein the interconnected pore structure is formed from a plurality of interconnected pores having a median diameter from about 20 microns to about 2,000 microns

Aspect 12: The foam biomaterial of any one of the preceding aspects, wherein the foam biomaterial has a density from about 0.01 grams per milliliter to about 1.5 grams per milliliter.

Aspect 13: The foam biomaterial of any one of the preceding aspects, wherein the foam biomaterial has a complex viscosity from about 1 Pa·s to about 10 Pa·s at 30 degrees Celsius.

Aspect 14: A method of making the foam biomaterial as in any one of the preceding aspects, the method comprising:
mechanically mixing a solution with a gas to generate a foam, wherein the solution comprises at least one biomaterial; and
depositing the foam in a selected location.

Aspect 15: The method of aspect 14, wherein mechanically mixing the solution comprises stirring the solution, vortex mixing, shaking, acoustic actuation, or mechanically introducing air bubbles into the solution, or a combination thereof.

Aspect 16: The method of aspect 14 or aspect 15, wherein the at least one biomaterial comprises a polymer, a protein, or a combination thereof.

Aspect 17: The method of any one of aspects 14-16, wherein the solution comprises at least one additive, the at least one additive comprising nanoparticles, microparticles, nanofibers, microfibers, an antibacterial compound, an antibiotic, a bioceramic, a ceramic, oxygen-generating material, at least one vitamin, at least one lipid, at least one phospholipid, at least one fatty acid, a biological factor, a polysaccharide, a nucleic acid, a growth factor, hydroxyapatite, calcium phosphate, dopamine-based material, carbon nanotubes, a Quaternary ammonium compound, polyhexamethylene biguanide (PHMB), methacryloyloxydodecylpyridinium bromide (MDPB), graphene, graphene oxide, a carbon derived material, liquid crystals, peptides, chitosan, silver-based materials, platelet-rich plasma, a blood-derived material, or a combination thereof.

Aspect 18: The method of any one of aspects 14-17, wherein the gas is air.

Aspect 19: The method of any one of aspects 14-18, wherein the gas comprises at least 50% oxygen.

Aspect 20: The method of any one of aspects 14-19, further comprising, prior to depositing the foam, maintaining the foam at a constant or substantially constant temperature.

Aspect 21: The method of any one of aspects 14-20, wherein depositing the foam and mechanically mixing the solution with the gas to generate the foam is performed in a single step.

Aspect 22: The method of any one of aspects 14-21, further comprising depositing the foam in a deposition device prior to depositing the foam in the selected location.

Aspect 23: The method of any one of aspects 14-22, wherein depositing the foam in the selected location comprises depositing the foam in or on a body of a patient in situ.

Aspect 24: The method of aspect 23, wherein depositing the foam in or on the body of the patient comprises depositing the foam in or on a wound of the body of the patient.

Aspect 25: The method of aspect 23, wherein the depositing the foam in or on the body of the patient comprises depositing the foam in the body of the patient through a minimally invasive surgery method, wherein the minimally invasive surgery method is one of laparoscopy, endoscopy, needle injection, intradermal delivery through needle arrays, or catheter-based delivery.

Aspect 26: The method of aspect 23, wherein depositing the foam in or on the body of the patient comprises depositing the foam in or on one of a head, a torso, or at least one limb of the body of the patient.

Aspect 27: The method of any one of aspects 14-26, further comprising crosslinking, after depositing the foam in the selected location, the foam to form the foam biomaterial having the interconnected pore structure.

Aspect 28: The method of aspect 27, wherein crosslinking the foam comprises crosslinking the foam with physical, mechanical, thermal, ionic, enzymatic, radiative crosslinking, or a combination thereof.

Aspect 29: The method of any one of aspects 14-29, further comprising adding cells to the solution.

Aspect 30: The method of aspect 29, wherein adding cells to the solution comprises adding cells to the foam.

Aspect 31: The method of any one of aspects 14-30, wherein depositing the foam in the selected location comprises depositing the foam to form a structure having a selected geometry.

Aspect 32: The method of aspect 30, further comprising placing the structure having the selected geometry in or on a patient.

Aspect 33: The method of any one of aspects 14-32, wherein depositing the foam in the selected location comprises depositing the foam with a 3D printer.

Aspect 34: The method of any one of aspects 14-33, wherein the solution does not comprise a sacrificial material.

Aspect 35: The method of any one of aspects 14-34, wherein the solution comprises an emulsifier.

Aspect 36: The method of any one of aspects 14-35, wherein the solution

comprises a surfactant, a foam booster, or both a surfactant and a foam booster.

Aspect 37: The method of any one of aspects 14-36, wherein mixing the solution comprises mixing the solution to provide a tailored porosity.

Aspect 38: The method of aspect 37, wherein mixing the solution to provide the tailored porosity comprises mechanically agitating the solution for a predetermined time and at a predetermined speed.

Aspect 39: The method of any one of aspects 14-38, wherein mechanically agitating the solution comprises mechanically agitating the solution in a syringe barrel, wherein the method further comprises inserting a plunger in the syringe barrel.

Aspect 40: The method of any one of aspects 14-39, wherein, the foam has an interconnected pore structure after deposition.

Aspect 41: The method of any one of aspects 14-40, wherein the solution comprises a hydrogel concentration from about 1% to about 30% w/v.

Aspect 42: A system for making the foam biomaterial as in any one of aspects 1-13, the system comprising:
a vessel:
a solution in the vessel, wherein the solution comprises a biomaterial; and
a mixer that is configured to combine the solution with a gas to generate a foam.

Aspect 43: The system of aspect 42, wherein the vessel is a syringe barrel, wherein the mixer is positioned within the syringe barrel.

Aspect 44: The system of aspect 42 or aspect 43, wherein the mixer comprises a stirring element.

Aspect 45: The system of aspect 44, wherein the mixer further comprises a motor that is coupled to the stirring element and is configured to cause the stirring element to rotate.

Aspect 46: The system of aspect 42, wherein the mixer is a static mixer.

Aspect 47: The system of aspect 42, wherein the mixer comprises a vortex mixer, a shaker, an acoustic actuator, or a bubbler.

Aspect 48: The system of any one of aspects 42-47, further comprising a deposition device comprising:

a nozzle defining an outlet: and an actuator that is configured to extrude the foam through the outlet of the nozzle.

Aspect 49: The system of aspect 48, wherein the deposition device is configured to be handheld.

Aspect 50: The system of aspect 48, wherein the deposition device comprises a robotic arm that is configured to position the nozzle.

Aspect 51: The system of aspect 48, wherein the deposition device further comprises one of: an endoscopy, a needle injector, an intradermal needle array, or a catheter.

Aspect 52: The system of aspect 48, wherein the deposition device comprises a controller, a temperature sensor in communication with the controller, and a heater in communication with the controller, wherein the heater is configured to maintain the foam at or substantially at a selected temperature.

Aspect 53: A foam biomaterial formed according to the method as in any one of aspects 14-41.

All references listed or cited in this disclosure, including those provided in the following reference lists, are hereby incorporated by reference in their entireties.

### REFERENCES FOR EXAMPLE 2

1. Tamayol A, Akbari M, Annabi N, Paul A, Khademhosseini A, Juncker D. Fiber-based tissue engineering: Progress, challenges, and opportunities. Biotechnol Adv. 2013:31: 669 687.
2. Naseer S M, Manbachi A, Samandari M, Walch P, Gao Y, Zhang YS, et al. Surface acoustic waves induced micropatterning of cells in gelatin methacryloyl (Ge1MA) hydrogels. Biofabrication. 2017:9: 015020.
3. Abudula T, Gauthaman K, Mostafavi A, Alshahrie A, Salah N, Morganti P, et al. Sustainable drug release from polycaprolactone coated chitin-lignin gel fibrous scaffolds. Sci Rep. 2020:10: 20428.
4. Mostafavi A, Daemi H, Rajabi S, Baharvand H. Highly tough and ultrafast self-healable dual physically crosslinked sulfated alginate-based polyurethane elastomers for vascular tissue engineering. Carbohydr Polym. 2021:257: 117632.
5. Akbari M, Tamayol A, Bagherifard S, Serex L, Mostafalu P, Faramarzi N, et al. Textile Technologies and Tissue Engineering: A Path Toward Organ Weaving. Adv Healthc Mater. 2016:5: 751-766.
6. [No title]. [cited 25 Jan. 2021]. Available:
   https://doi.org/10.1021/acsmaterialslett.0c00224
7. Kang H-W, Lee S J, Ko I K, Kengla C, Yoo J J, Atala A. A 3D bioprinting system to produce human-scale tissue constructs with structural integrity. Nat Biotechnol. 2016:34: 312-319.
8. Farzin A, Miri A K, Sharifi F, Faramarzi N, Jaberi A, Mostafavi A, et al. 3D-Printed Sugar-Based Stents Facilitating Vascular Anastomosis. Adv Healthc Mater. 2018:7: e1800702.
9. Murphy SV, Atala A. 3D bioprinting of tissues and organs. Nat Biotechnol. 2014:32: 773-785.
10. Chávez-Madero C, de León-Derby M D, Samandari M, Ceballos-González C F, Bolívar-Monsalve E J, Mendoza-Buenrostro C, et al. Using chaotic advection for facile high-throughput fabrication of ordered multilayer micro- and nanostructures: continuous chaotic printing. Biofabrication. 2020:12: 035023.
11. Gerdes S, Mostafavi A, Ramesh S, Memic A, Rivero IV, Rao P, et al. Process-Structure-Quality Relationships of Three-Dimensional Printed Poly(Caprolactone)-Hydroxyapatite Scaffolds. Tissue Eng Part A. 2020:26: 279-291.
12. Li H, Tan C, Li L. Review of 3D printable hydrogels and constructs. Materials & Design. 2018. pp. 20-38, doi:10.1016/j.matdes.2018.08.023
13. Nanocomposite hydrogels for tissue engineering applications. Biomaterials for Organ and Tissue Regeneration. Woodhead Publishing: 2020. pp. 499-528.
14. Sun M, Sun X, Wang Z, Guo S, Yu G, Yang H. Synthesis and Properties of Gelatin Methacryloyl (Ge1MA) Hydrogels and Their Recent Applications in Load-Bearing Tissue. Polymers. 2018: 10, doi: 10.3390/polym10111290
15. Miri A K, Hosseinabadi H G, Cecen B, Hassan S, Zhang Y S. Permeability mapping of gelatin methacryloyl hydrogels. Acta Biomater. 2018:77: 38-47.
16. Annabi N, Tamayol A, Uquillas J A, Akbari M, Bertassoni L E, Cha C, et al. 25^{th} anniversary article: Rational design and applications of hydrogels in regenerative medicine. Adv Mater. 2014:26: 85-123.
17. Elkhoury K, Russell C S, Sanchez-Gonzalez L, Mostafavi A, Williams T J, Kahn C, et al. Soft-Nanoparticle Functionalization of Natural Hydrogels for Tissue Engineering Applications. Adv Healthc Mater. 2019:8: e1900506.
18. Lieleg O, Ribbeck K. Biological hydrogels as selective diffusion barriers. Trends Cell Biol. 2011:21: 543-551.
19. Verbeke CS, Mooney DJ. Injectable, Pore-Forming Hydrogels for In Vivo Enrichment of Immature Dendritic Cells. Adv Healthc Mater. 2015:4: 2677-2687.
20. Keskar V, Marion N W, Mao J J, Gemeinhart R A. In vitro evaluation of macroporous hydrogels to facilitate stem cell infiltration, growth, and mineralization. Tissue Eng Part A. 2009:15: 1695-1707.
21. Zeltinger J, Sherwood J K, Graham D A, Mueller R, Griffith L G. Effect of pore size and void fraction on cellular adhesion, proliferation, and matrix deposition. Tissue Eng. 2001:7: 557-572.
22. He Y, Yang F, Zhao H, Gao Q, Xia B, Fu J. Research on the printability of hydrogels in 3D bioprinting. Scientific Reports. 2016, doi: 10.1038/srep29977
23. Nava M M, Draghi L, Giordano C, Pietrabissa R. The effect of scaffold pore size in cartilage tissue engineering. J Appl Biomater Funct Mater. 2016:14: e223-9.
24. Griffith LG. Tissue Engineering—Current Challenges and Expanding Opportunities. Science. 2002. pp. 1009-1014, doi: 10.1126/science. 1069210
25. Direct 3D bioprinting of prevascularized tissue constructs with complex microarchitecture. Biomaterials. 2017:124: 106-115.
26. Huebsch N, Lippens E, Lee K, Mehta M, Koshy S T, Damell M C, et al. Matrix elasticity of void-forming hydrogels controls transplanted-stem-cell-mediated bone formation. Nat Mater. 2015:14: 1269-1277.
27. Autissier A, Le Visage C, Pouzet C, Chaubet F, Letourneur D. Fabrication of porous polysaccharide-based scaffolds using a combined freeze-drying/cross-linking process. Acta Biomater. 2010; 6: 3640-3648.
28. Ji C, Annabi N, Khademhosseini A, Dehghani F. Fabrication of porous chitosan scaffolds for soft tissue engineering using dense gas CO2. Acta Biomater. 2011:7: 1653-1664.
29. Lee S B, Kim Y H, Chong M S, Hong S H, Lee Y M. Study of gelatin-containing artificial skin V: fabrication of gelatin scaffolds using a salt-leaching method. Biomaterials. 2005:26: 1961-1968.
30. Van Vlierberghe S, Graulus G-J, Keshari Samal S, Van Nieuwenhove I, Dubruel P. Porous hydrogel biomedical foam scaffolds for tissue repair. Biomedical Foams for Tissue Engineering Applications. 2014. pp. 335-390, doi: 10.1533/9780857097033.2.335
31. Netti P. Biomedical Foams for Tissue Engineering Applications. Elsevier: 2014.
32. Shao L, Gao Q, Xie C, Fu J, Xiang M, Liu Z, et al. Sacrificial microgel-laden bioink-enabled 3D bioprinting of mesoscale pore networks. Bio-Design and Manufacturing. 2020. pp. 30-39, doi: 10.1007/s42242-020-00062-y
33. Ying G-L, Jiang N, Maharjan S, Yin Y-X, Chai R-R, Cao X, et al. Aqueous Two-Phase Emulsion Bioink-Enabled 3D Bioprinting of Porous Hydrogels. Adv Mater. 2018:30: e1805460.
34. Hori A, Watabe Y, Yamada M, Yajima Y, Utoh R, Seki M. One-Step Formation of Microporous Hydrogel Sponges Encapsulating Living Cells by Utilizing Bicontinuous Dispersion of Aqueous Polymer Solutions. ACS Applied Bio Materials. 2019. pp. 2237-2245, doi: 10.1021/acsabm.9b00194
35. Salvador T, Oliveira M B, Mano J F. Leachable-Free Fabrication of Hydrogel Foams Enabling Homogeneous Viability of Encapsulated Cells in Large-Volume Constructs. Adv Healthc Mater. 2020:9: e2000543.
36. Yue K, Trujillo-de Santiago G, Alvarez M M, Tamayol A, Annabi N, Khademhosseini A. Synthesis, properties, and biomedical applications of gelatin methacryloyl (Ge1MA) hydrogels. Biomaterials. 2015:73: 254-271.
37. Russell C S, Mostafavi A, Quint JP, Panayi A C, Baldino K, Williams T J, et al. In Situ Printing of Adhesive Hydrogel Scaffolds for the Treatment of Skeletal Muscle Injuries. ACS Applied Bio Materials. 2020. pp. 1568-1579, doi: 10.1021/acsabm.9b01176
38. Mendizabal E, Castellanos-Ortega J R, Puig J E. A method for selecting a polyvinyl alcohol as stabilizer in suspension polymerization. Colloids and Surfaces. 1992. pp. 209-217, doi: 10.1016/0166-6622(92)80242-t
39. Tran P A, Nguyen H T, Fox K, Tran N. In vitro cytotoxicity of iron oxide nanoparticles: effects of chitosan and polyvinyl alcohol as stabilizing agents. Materials Research Express. 2018. p. 035051, doi: 10.1088/2053-1591/aab5f3
40. Website. [cited 11 Jan. 2021], doi: 10.1007/s11340-018-00456-1
41. Website. [cited 10 Feb. 2021]. Available: Ying, G., Jiang, N., Yu, C. et al. Three-dimensional bioprinting of gelatin methacryloyl (Ge1MA). Bio-des. Manuf. 1, 215-224 (2018). https://doi.org/10.1007/s42242-018-0028-8
42. Annabi N, Rana D, Shirzaei Sani E, Portillo-Lara R, Gifford J L, Fares MM, et al. Engineering a sprayable and elastic hydrogel adhesive with antimicrobial properties for wound healing. Biomaterials. 2017: 139: 229-243.
43. Heinrich M A, Liu W, Jimenez A, Yang J, Akpek A, Liu X, et al. 3D Bioprinting: from Benches to Translational Applications. Small. 2019:15: e1805510.
44. One-step wettability patterning of PDMS microchannels for generation of monodisperse alginate microbeads by in Situ external gelation in double emulsion microdroplets. Sens Actuators B Chem. 2019:291: 418-425.
45. Markstedt K, Mantas A, Tournier I, Martinez Avila H, Hägg D, Gatenholm P. 3D Bioprinting Human Chondrocytes with Nanocellulose-Alginate Bioink for Cartilage Tissue Engineering Applications. Biomacromolecules. 2015:16: 1489-1496.
46. Gao Q, Niu X, Shao L, Zhou L, Lin Z, Sun A, et al. 3D printing of complex Ge1MA-based scaffolds with nanoclay. Biofabrication. 2019; 11: 035006.
47. Lee A, Hudson A R, Shiwarski D J, Tashman J W, Hinton T J, Yerneni S, et al. 3D bioprinting of collagen to rebuild components of the human heart. Science. 2019:365: 482-487.
48. Shao L, Gao Q, Zhao H, Xie C, Fu J, Liu Z, et al. Fiber-Based Mini Tissue with Morphology-Controllable Ge1MA Microfibers. Small. 2018:14: e1802187.
49. Kolesky D B, Truby R L, Gladman A S, Busbee T A, Homan K A, Lewis J A. 3D bioprinting of vascularized, heterogeneous cell-laden tissue constructs. Adv Mater. 2014:26: 3124-3130.
50. Tamayol A, Najafabadi A H, Aliakbarian B, Arab-Tehrany E, Akbari M, Annabi N, et al. Hydrogel Templates for Rapid Manufacturing of Bioactive Fibers and 3D Constructs. Adv Healthc Mater. 2015:4: 2146-2153.
51. Colosi C, Shin S R, Manoharan V, Massa S, Costantini M, Barbetta A, et al. Microfluidic Bioprinting of Heterogeneous 3D Tissue Constructs Using Low-Viscosity Bioink. Adv Mater. 2016:28: 677-684.
52. Yin J, Yan M, Wang Y, Fu J, Suo H. 3D Bioprinting of Low-Concentration Cell-Laden Gelatin Methacrylate (Ge1MA) Bioinks with a Two-Step Cross-linking Strategy. ACS Appl Mater Interfaces. 2018: 10: 6849-6857.
53. Griffin D R, Weaver W M, Scumpia P O, Di Carlo D, Segura T. Accelerated wound healing by injectable microporous gel scaffolds assembled from annealed building blocks. Nat Mater. 2015:14: 737-744.
54. Gholobova D, Terrie L, Gerard M, Declercq H, Thorrez L. Vascularization of tissue-engineered skeletal muscle constructs. Biomaterials. 2020:235: 119708.
55. Effect of stirring rate on microstructure and properties of microporous mullite ceramics. J Mater Process Technol. 2018:261: 159-163.
56. Xu C, Wang H, Wang D, Zhu X, Zhu Y, Bai X, et al. Improvement of Foaming Ability of Surfactant Solutions by Water-Soluble Polymers: Experiment and Molecular Dynamics Simulation. Polymers. 2020; 12: 571.
57. New insight into foam stability enhancement mechanism, using polyvinyl alcohol (PVA) and nanoparticles. J Mol Liq. 2020:307: 112755.
58. Error Cookies Turned Off. [cited 28 Jan. 2021]. Available:
   https://doi.org/10.1002/adfm.202003740
59. Woodard J R, Hilldore A J, Lan S K, Park C J, Morgan A W, Eurell J A C, et al. The mechanical properties and osteoconductivity of hydroxyapatite bone scaffolds with multi-scale porosity. Biomaterials. 2007:28: 45-54.
60. Committee F, F04 Committee. Test Method for Strength Properties of Tissue Adhesives in Lap-Shear by Tension Loading, doi: 10.1520/f2255
61. Panayi A C, Smit L, Hays N, Udeh K, Endo Y, Li B, et al. A porous collagen-GAG scaffold promotes muscle regeneration following volumetric muscle loss injury. Wound Repair and Regeneration. 2020. pp. 61-74, doi: 10.1111/wrr. 12768

### REFERENCES FOR EXAMPLE 3

1 Samandari. M. et al. Bioinks and bioprinting strategies for skeletal muscle tissue engineering Advanced Materials (2022).
2 Rivas. D. A. & Fielding. R. A. in Encyclopedia of Human Nutrition (Third Edition) (ed Benjamin Caballero) 193-199 (Academic Press. 2013).
3 Hall. J. Guyton and Hall Textbook of Medical Physiology. 13^{th} edn. (Elsevier, 2015).
4 Relaix. F. et al. Perspectives on skeletal muscle stem cells. Nature Communications 12. 1-11 (2021).
5. Endo. Y. et al. Loss of ARNT in skeletal muscle limits muscle regeneration in aging. The FASEB Journal 34. 16086-16104 (2020).
6 Järvinen. T. A. H., Järvinen. T. L. N., Kääriäinen. M., Kalimo. H. & Järvinen. M. Muscle Injuries: Biology and Treatment. The American Journal of Sports Medicine 33. 745-764, doi: 10.1177/0363546505274714 (2005).
7 Wang. Y. X. & Rudnicki. M. A. Satellite cells, the engines of muscle repair. Nature Reviews Molecular Cell Biology 13. 127-133, doi: 10.1038/nrm3265 (2012).
8 Tidball. J. G. Regulation of muscle growth and regeneration by the immune system. Nature Reviews Immunology 17. 165-178, doi: 10.1038/nri.2016.150 (2017).
9 Endo. Y., Nourmahnad. A. & Sinha. I. Optimizing skeletal muscle anabolic response to resistance training in aging. Frontiers in physiology 11. 874 (2020).
10. Grogan. B. F. & Hsu. J. R. Volumetric muscle loss. J Am Acad Orthop Surg 19 Suppl 1. S35-37, doi: 10.5435/00124635-201102001-00007 (2011).
11 Corona. B. T., Rivera. J. C., Owens. J. G., Wenke. J. C. & Rathbone. C. R. Volumetric muscle loss leads to permanent disability following extremity trauma. Journal of Rehabilitation Research & Development 52 (2015).
12 Nuutila. K. et al. Gene expression profiling of skeletal muscle after volumetric muscle loss. Wound Repair and Regeneration 25. 408-413, doi:https://doi.org/10.1111/wrr. 12547 (2017).
13 Grasman. J. M., Zayas. M. J., Page. R. L. & Pins. G. D. Biomimetic scaffolds for regeneration of volumetric muscle loss in skeletal muscle injuries. Acta Biomater 25, 2-15, doi:10.1016/j.actbio.2015.07.038 (2015).
14 Ulusal. A. E., Lin. C.-H., Lin. Y.-T., Ulusal. B. G. & Yazar. S. The use of free flaps in the management of type IIIB open calcaneal fractures. Plastic and reconstructive surgery 121. 2010-2019 (2008).
15 Doi. K., Hattori. Y., Tan. S.-H. & Dhawan. V. Basic science behind functioning free muscle transplantation. Clinics in plastic surgery 29. 483-495 (2002).
16 Lin. C.-H., Lin. Y.-T., Yeh. J.-T. & Chen. C.-T. Free functioning muscle transfer for lower extremity posttraumatic composite structure and functional defect. Plastic and reconstructive surgery 119. 2118-2126 (2007).
17 Greising. S. M. et al. Early rehabilitation for volumetric muscle loss injury augments endogenous regenerative aspects of muscle strength and oxidative capacity. BMC musculoskeletal disorders 19. 1-11 (2018).
18 Gilbert-Honick. J. & Grayson. W. Vascularized and innervated skeletal muscle tissue engineering. Advanced Healthcare Materials 9. 1900626 (2020).
19 Costantini. M. et al. in Biofabrication and 3D Tissue Modeling 184-215 (2019).
20 Zhuang. P., An. J., Chua. C. K. & Tan. L. P. Bioprinting of 3D in vitro skeletal muscle models: A review. Materials & Design 193. 108794, doi: https://doi. org/10.1016/j.matdes. 2020.108794 (2020).
21 Ostrovidov. S. et al. 3D Bioprinting in Skeletal Muscle Tissue Engineering. Small 15. 1805530, doi: 10.1002/smll.201805530 (2019).
22 Murphy. S. V. & Atala. A. 3D bioprinting of tissues and organs. Nature Biotechnology 32. 773-785, doi: 10.1038/nbt.2958 (2014).
23 Samandari. M., Quint. J. & Tamayol. A. in Musculoskeletal Tissue Engineering (ed Yupeng Chen) 167-200 (Elsevier. 2022).
24 Nuutila. K. et al. In vivo printing of growth factor-eluting adhesive scaffolds improves wound healing. Bioactive Materials, doi:https://doi.org/10.1016/j.bioactmat.2021.06.030 (2022).
25 Singh. S., Choudhury. D., Yu. F., Mironov. V. & Naing. M. W. In situ bioprinting-Bioprinting from benchside to bedside? Acta Biomaterialia 101. 14-25, doi: https://doi.org/10. 1016/j.actbio. 2019.08.045 (2020).
26 Albanna. M. et al. In Situ Bioprinting of Autologous Skin Cells Accelerates Wound Healing of Extensive Excisional Full-Thickness Wounds. Scientific Reports 9. 1856, doi: 10.1038/s41598-018-38366-w (2019).
27 Mostafavi. A. et al. In situ printing of scaffolds for reconstruction of bone defects. Acta Biomaterialia 127. 313-326, doi:https://doi.org/10.1016/j.actbio. 2021.03.009 (2021).
28 Quint. J. P. et al. In Vivo Printing of Nanoenabled Scaffolds for the Treatment of Skeletal Muscle Injuries. Advanced Healthcare Materials n/a. 2002152, doi:https://doi.org/10.1002/adhin.202002152 (2021).
29 Russell. C. S. et al. In Situ Printing of Adhesive Hydrogel Scaffolds for the Treatment of Skeletal Muscle Injuries. AC'S Applied Bio Materials 3. 1568-1579, doi: 10.1021/acsabm.9b01176 (2020).
30 Mostafavi. A. et al. Colloidal multiscale porous adhesive (bio)inks facilitate scaffold integration. Applied Physics Reviews 8. 041415, doi: 10.1063/5.0062823 (2021).
31 Mulbauer. G. D. & Matthew. H. W. Biomimetic scaffolds in skeletal muscle regeneration. Discoveries 7 (2019).
32 Panayi. A. C. et al. A porous collagen-GAG scaffold promotes muscle regeneration following volumetric muscle loss injury. Wound Repair and Regeneration 28. 61-74 (2020).
33 Verbeke. C. S. & Mooney. D. J. Injectable. Pore-Forming Hydrogels for In Vivo Enrichment of Immature Dendritic Cells. Adv. Healthc. Mater. 4. 2677-2687, doi: 10.1002/adhm.201500618 (2015).
34 Keskar. V., Marion. N. W., Mao. J. J. & Gemeinhart. R. A. In vitro evaluation of macroporous hydrogels to facilitate stem cell infiltration, growth, and mineralization. Tissue Eng. Part A 15. 1695-1707, doi: 10.1089/ten.tea.2008.0238 (2009).
35 Lieleg. O. & Ribbeck. K. Biological hydrogels as selective diffusion barriers. Trends Cell Biol. 21. 543-551, doi: 10.1016/j.tcb.2011.06.002 (2011).
36 Sicari. B. M. et al. An Acellular Biologic Scaffold Promotes Skeletal Muscle Formation in Mice and Humans with Volumetric Muscle Loss. Science Translational Medicine 6. 234ra258-234ra258, doi:doi: 10.1126/scitranslmed.3008085 (2014).
37 Xu. Y. et al. Melatonin-Based and Biomimetic Scaffold as Muscle-ECM Implant for Guiding Myogenic Differentiation of Volumetric Muscle Loss. Advanced Functional Materials 30. 2002378, doi:https:/doi.org/10.1002/adfin.202002378 (2020).
38 Kim. J. H. et al. Neural cell integration into 3D bioprinted skeletal muscle constructs accelerates restoration of muscle function. Nature Communications 11. 1025, doi: 10.1038/s41467-020-14930-9 (2020).
39 Kim. J. H. et al. 3D Bioprinted Human Skeletal Muscle Constructs for Muscle Function Restoration. Scientific Reports 8. 12307, doi: 10.1038/s41598-018-29968-5 (2018).
40 Karalaki. M., Fili. S., Philippou. A. & Koutsilicris. M. Muscle regeneration: cellular and molecular events. In vivo 23. 779-796 (2009).
41 Quint. J. P. et al. Nanoengineered myogenic scaffolds for skeletal muscle tissue engineering. Nanoscale 14. 797-814, doi: 10.1039/DINR06143G (2022).
42 Ahmad. S. S., Ahmad. K., Lec. E. J., Lec. Y.-H. & Choi, I. Implications of Insulin-Like Growth Factor-1 in Skeletal Muscle and Various Diseases. Cells 9. 1773, doi: 10.3390/cells9081773 (2020).
43 Philippou. A. & Barton. E. R. Optimizing IGF-I for skeletal muscle therapeutics. Growth Horm IGF Res 24. 157-163, doi: 10.1016/j.ghir.2014.06.003 (2014).
44 Lec. K., Silva. E. A. & Mooney. D. J. Growth factor delivery-based tissue engineering: general approaches and a review of recent developments. Journal of the Royal Society Interface 8. 153-170 (2011).
45 Quarta. M. et al. Bioengineered constructs combined with exercise enhance stem cell-mediated treatment of volumetric muscle loss. Nature communications 8. 1-17 (2017).
46 Samandari. M. et al. Controlling cellular organization in bioprinting through designed 3D microcompartmentalization. Applied Physics Reviews 8. 021404, doi: 10.1063/5.0040732 (2021).
47 Nascer. S. M. et al. Surface acoustic waves induced micropatterning of cells in gelatin methacry loyl (Ge1MA) hydrogels. Biofabrication 9. 015020, doi: 10.1088/1758-5090/aa585e (2017).
48 Wang. H. et al. Oppositely Charged Gelatin Nanospheres as Building Blocks for Injectable and Biodegradable Gels. Advanced Materials 23. H119-H124, doi: https://doi. org/10.1002/adma.201003908 (2011).
49 Mullen. L. M. et al. Binding and release characteristics of insulin-like growth factor-1 from a collagen-glycosaminoglycan scaffold. Tissue Engineering Part C: Methods 16. 1439-1448 (2010).
50 Tomczak. K. K. et al. Expression profiling and identification of novel genes involved in myogenic differentiation. The FASEB journal 18. 1-23 (2004).
51 Engler. A. J., Sen. S., Sweeney. H. L. & Discher. D. E. Matrix Elasticity Directs Stem Cell Lineage Specification. Cell 126. 677-689, doi:https://doi.org/10.1016/j.cell.2006.06.044 (2006).
52 Kammoun. M. et al. Development of a novel multiphysical approach for the characterization of mechanical properties of musculotendinous tissues. Scientific Reports 9. 7733, doi: 10.1038/s41598-019-44053-1 (2019).
53 Ouyang. L., Yao. R., Zhao. Y. & Sun. W. Effect of bioink properties on printability and cell viability for 3D bioplotting of embryonic stem cells. Biofabrication 8. 035020, doi: 10.1088/1758-5090/8/3/035020 (2016).
54 Yuc. K. et al. Synthesis, properties, and biomedical applications of gelatin methacry loyl (Ge1MA) hydrogels. Biomaterials 73. 254-271, doi:https://doi.org/10.1016/j.biomaterials.2015.08.045 (2015).
55 Rahimi Mamaghani, K., Morteza Naghib. S., Zahedi, A. & Mozafari. M. Synthesis and microstructural characterization of GelMa/PEGDA hybrid hydrogel containing graphene oxide for biomedical purposes. Materials Today: Proceedings 5. 15635-15644, doi:https://doi.org/10.1016/j.matpr.2018.04.173 (2018).
56 Wilde. P., Mackie. A., Husband. F., Gunning. P. & Morris. V. Proteins and emulsifiers at liquid interfaces. Advances in Colloid and Interface Science 108-109. 63-71, doi:https://doi.org/10.1016/j.cis.2003.10.011 (2004).
57 Eggermont. L. J., Rogers. Z. J., Colombani. T., Memic. A. & Bencherif. S. A. Injectable Cryogels for Biomedical Applications. Trends in Biotechnology 38. 418-431, doi:https://doi.org/10.1016/j.tibtech. 2019.09.008 (2020).
58 Huebsch. N. et al. Matrix elasticity of void-forming hydrogels controls transplanted-stem-cell-mediated bone formation. Nat. Mater. 14. 1269-1277, doi: 10.1038/nmat4407 (2015).
59 Engler. A. J. et al. Myotubes differentiate optimally on substrates with tissue-like stiffness: pathological implications for soft or stiff microenvironments. Journal of Cell Biology 166. 877-887, doi: 10.1083/jcb.200405004 (2004).
60 Lin. S., Liu. J., Liu. X. & Zhao. X. Muscle-like fatigue-resistant hydrogels by mechanical training. Proceedings of the National Academy of Sciences 116. 10244-10249 (2019).
61 Shirzaci Sani. E. et al. Sutureless repair of corneal injuries using naturally derived bioadhesive hydrogels. Science Advances 5, caav1281, doi: 10.1126/sciadv.aav1281 (2019).
62 Assmann. A. et al. A highly adhesive and naturally derived sealant. Biomaterials 140. 115-127, doi:https://doi. org/10.1016/j biomaterials.2017.06.004 (2017).
63 Yoshida. T. & Delafontaine. P. Mechanisms of IGF-1-mediated regulation of skeletal muscle hypertrophy and atrophy. Cells 9. 1970 (2020).
64 Carroll. P. V. et al. Recombinant human insulin-like growth factor-I (rhIGF-I) therapy in adults with type 1 diabetes mellitus: effects on IGFs. IGF-binding proteins, glucose levels and insulin treatment. Clinical Endocrinology 49. 739-746, doi:https://doi.org/10.10464.1365_2265.1998.00600.x (1998).
65 Jenkins. P. J. Growth hormone and exercise. Clin Endocrinol (Oxf) 50. 683-689, doi: 10.1046/j.1365-2265.1999.00784.x (1999).
66 Tidball. J. G. & Villalta. S. A. Regulatory interactions between muscle and the immune system during muscle regeneration. American Journal of Physiology-Regulatory, Integrative and Comparative Physiology 298. R1173-R1187, doi: 10.1152/ajpregu. 00735.2009 (2010).
67 Dalske. K. A. et al. Independent of physical activity. volumetric muscle loss injury in a murine model impairs whole-body metabolism. Plos one 16. e0253629 (2021).
68 Greising. S. M., Corona. B. T., McGann. C., Frankum, J. K. & Warren. G. L. Therapeutic approaches for volumetric muscle loss injury: a systematic review and meta-analysis. Tissue Engineering Part B: Reviews 25. 510-525 (2019).
69 Langridge. B., Griffin. M. & Butler. P. E. Regenerative medicine for skeletal muscle loss: a review of current tissue engineering approaches. Journal of Materials Science: Materials in Medicine 32. 1-16 (2021).
70 Greising. S. M. et al. Unwavering pathobiology of volumetric muscle loss injury. Scientific reports 7. 1-14 (2017).
71 Panayi. A. C. et al. A porous collagen-GAG scaffold promotes muscle regeneration following volumetric muscle loss injury. Wound Repair and Regeneration 28. 61-74, doi: 10.1111/wrr.12768 (2020).
72 Aurora. A., Garg. K., Corona. B. T. & Walters. T. J. Physical rehabilitation improves muscle function following volumetric muscle loss injury. BMC sports science, medicine and rehabilitation 6. 1-10 (2014).
73 Adams. G. R. Role of insulin-like growth factor-I in the regulation of skeletal muscle adaptation to increased loading. Exercise and sport sciences reviews 26. 31-60 (1998).
74 Schwarz. A. J., Brasel. J., Hintz. R. L., Mohan. S. & Cooper. D. Acute effect of brief low-and high-intensity exercise on circulating insulin-like growth factor (IGF) I, II, and IGF-binding protein-3 and its proteolysis in young healthy men. The Journal of Clinical Endocrinology & Metabolism 81. 3492-3497 (1996).
75 Garg. K. et al. Volumetric muscle loss: persistent functional deficits beyond frank loss of tissue. Journal of Orthopaedic Research 33. 40-46 (2015).
76 Olfert. I. M., Baum. O., Hellsten. Y. & Egginton. S. Advances and challenges in skeletal muscle angiogenesis. American journal of physiology-heart and circulatory physiology 310. H326-H336 (2016).
77 (ASTM Internatational. West Conshohocken, P A. 2003).

Although the foregoing invention has been described in some detail by way of illustration and example for purposes of clarity of understanding. certain changes and modifications may be practiced within the scope of the appended claims.

What is claimed is:

1. A foam biomaterial comprising:
a biomaterial ink having an interconnected pore structure, wherein the foam biomaterial has a porosity from about 10% to about 90%.

2. The foam biomaterial of claim 1, where the interconnected pore structure is formed without use of sacrificial materials.

3. The foam biomaterial of claim 1, comprising at least one of: a polymer, a protein, or a combination thereof.

4. The foam biomaterial of claim 1, further comprising at least one additive, the at least one additive comprising nanoparticles, microparticles, nanofibers, microfibers, an antibacterial compound, an antibiotic, a bioceramic, a ceramic, oxygen-generating material, at least one vitamin, at least one lipid, at least one phospholipid, at least one fatty acid, a biological factor, a polysaccharide, a nucleic acid, a growth factor, hydroxyapatite, calcium phosphate, dopamine-based material, carbon nanotubes, a Quaternary ammonium compound, polyhexamethylene biguanide (PHMB), methacryloyloxydodecylpyridinium bromide (MDPB), graphene, graphene oxide, a carbon derived material, liquid crystals, peptides, chitosan, silver-based materials, platelet-rich plasma, a blood-derived material, or a combination thereof.

5. The foam biomaterial of claim 4, wherein the additive is provided in a concentration from 0 to about 90% weight percent.

6. The foam biomaterial of claim 1, where the foam biomaterial is crosslinked or solidified through physical, ionic, thermal, chemical, enzymatic, photo crosslinking, or a combination thereof.

7. The foam biomaterial of claim 1, where the foam biomaterial is adhered to a medical device or an implantable material.

8. The foam biomaterial of claim 1, wherein the interconnected pore structure supports cellular infiltration, tissue remodeling, tissue regeneration, and/or tissue fidelity and stability.

9. The foam biomaterial of claim 1, wherein the foam biomaterial has a Young's modulus from 1 kPa to 100 MPa.

10. The foam biomaterial of claim 1, wherein the foam biomaterial is capable of sustaining 50% strain for 10 cycles without breaking.

11. The foam biomaterial of claim 1, wherein the interconnected pore structure is formed from a plurality of interconnected pores having a median diameter from about 20 microns to about 2,000 microns

12. The foam biomaterial of claim 1, wherein the foam biomaterial has a density from about 0.01 grams per milliliter to about 1.5 grams per milliliter.

13. The foam biomaterial of claim 1, wherein the foam biomaterial has a complex viscosity from about 1 Pa·s to about 10 Pa·s at 30 degrees Celsius.

14. A method of making a foam biomaterial, the method comprising:
mechanically mixing a solution with a gas to generate a foam, wherein the solution comprises at least one biomaterial; and
depositing the foam in a selected location,
wherein the foam biomaterial comprises a biomaterial ink having an interconnected pore structure, wherein the foam biomaterial has a porosity from about 10% to about 90%.

15. The method of claim 14, wherein mechanically mixing the solution comprises: stirring the solution, vortex mixing, shaking, acoustic actuation, mechanically introducing air bubbles into the solution, or a combination thereof.

16. (canceled)

17. The method of claim 14, wherein the solution comprises at least one additive, the at least one additive comprising nanoparticles, microparticles, nanofibers, microfibers, an antibacterial compound, an antibiotic, a bioceramic, a ceramic, oxygen-generating material, at least one vitamin, at least one lipid, at least one phospholipid, at least one fatty acid, a biological factor, a polysaccharide, a nucleic acid, a growth factor, hydroxyapatite, calcium phosphate, dopamine-based material, carbon nanotubes, a Quaternary ammonium compound, polyhexamethylene biguanide (PHMB), methacryloyloxydodecylpyridinium bromide (MDPB), graphene, graphene oxide, a carbon derived material, liquid crystals, peptides, chitosan, silver-based materials, platelet-rich plasma, a blood-derived material, or a combination thereof, and wherein the additive is provided in a concentration from 0 to about 90% weight percent.

18. (canceled)

19. (canceled)

20. (canceled)

21. The method of claim 14, wherein depositing the foam and mechanically mixing the solution with the gas to generate the foam is performed in a single step.

22. (canceled)

23. The method of claim 14, wherein depositing the foam in the selected location comprises depositing the foam in or on a body of a patient in situ.

24. (canceled)

25. (canceled)

26. (canceled)

27. The method of claim 14, further comprising crosslinking, after depositing the foam in the selected location, the foam to form the foam biomaterial having the interconnected pore structure.

28.-41. (canceled)

42. A system for making a foam biomaterial, the system comprising:
a vessel;
a solution in the vessel, wherein the solution comprises a biomaterial; and
a mixer that is configured to combine the solution with a gas to generate a foam.

43-53. (canceled)
